(19) United States
(12) Patent Application Publication
(10) Pub. No.: US 2008/0051416 A1
(43) Pub. Date: Feb. 28, 2008

(54) Novel Compounds

(75) Inventors: Jeffrey Charles Boehm, King of Prussia, PA (US); James Francis Callahan, King of Prussia, PA (US); Zehong Wan, King of Prussia, PA (US); Hongxing Yan, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SMITHKLINE BEECHAM CORPORATION, Philadelphia, PA (US)

(21) Appl. No.: 11/576,748

(22) PCT Filed: Oct. 5, 2005
(86) PCT No.: PCT/US05/35743

§ 371 Date: Apr. 5, 2007

Related U.S. Application Data
(60) Provisional application No. 60/616,065, filed on Oct. 5, 2004.
(60) Provisional application No. 60/719,729, filed on Sep. 22, 2005.

(51) Int. Cl. A61K 31/495; A61K 31/164; A61K 31/195; A61K 31/215; A61K 31/40; A61P 43/00; C07C 233/00; C07D 207/00; C07D 221/00; C07D 295/00; C07D 277/02; C07D 233/54; C07D 213/02; C07C 311/01; C07C 229/00; A61K 31/4164; A61K 31/426; A61K 31/44; A61K 31/445

(52) U.S. Cl. 514/254.02; 514/252.12; 514/255.01; 514/326; 514/327; 514/352; 514/371; 514/399; 514/428; 514/535; 514/563; 514/603; 514/616; 544/369; 544/383; 544/400; 546/208; 546/221; 546/309; 548/195; 548/338.1; 548/567; 560/19; 562/433; 564/153; 564/157; 564/86

(57) ABSTRACT

The present invention is directed to novel compounds of formula (I): 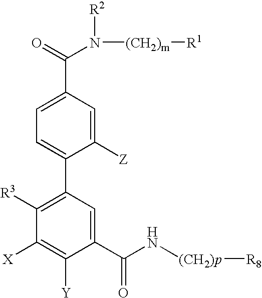 or pharmaceutically acceptable derivatives thereof, and their use as pharmaceuticals, particularly as p38 kinase inhibitors.

### SUMMARY OF THE INVENTION

This invention relates to novel compounds and their use as pharmaceuticals, particularly as p38 kinase inhibitors, for the treatment of certain diseases and conditions.

### BACKGROUND OF THE INVENTION

Intracellular signal transduction is the means by which cells respond to extracellular stimuli. Regardless of the nature of the cell surface receptor (e.g. protein tyrosine kinase or seven-transmembrane G-protein coupled), protein kinases and phosphatases along with phospholipases are the essential machinery by which the signal is further transmitted within the cell [Marshall, J. C. Cell, 80, 179-278 (1995)]. Protein kinases can be categorized into five classes with the two major classes being tyrosine kinases and serine/threonine kinases, depending upon whether the enzyme phosphorylates its substrate(s) on specific tyrosine(s) or serine/threonine(s) residues [Hunter, T., Methods in Enzymology (Protein Kinase Classification) p. 3, Hunter, T.; Sefton, B. M.; eds. vol. 200, Academic Press; San Diego, 1991].

Three major related intracellular pathways, the mitogen-activated kinases, or MAPKs, are now understood to transduce signals from many extracellular stimuli such as environmental stress, infectious agents, cytokines and growth factors. The MAPKs modulate the activity of numerous cell functions such as translocation and activation of transcription factors that control transcription of effector molecules such as cytokines, COX-2, iNOS; the activity of downstream kinases that effect translation of mRNAs; and cell cycle pathways through transcription or modification of enzymes. One of these three major pathways is the p38 MAPK pathway, which refers in most cell types to the isoform p38a which is ubiquitously expressed. The role of p38 in a multitude of functions, particularly related to inflammatory response has been elucidated using selective p38 inhibitors in numerous in vitro and in vivo studies. These functions have been extensively reviewed and a summary can be found in Nature Reviews [Kumar, S, Nature Rev. Drug Discovery, 2:717 (2003)]

Extracellular stimuli such as those described above are generated in a number of chronic diseases which are now understood to have a common underlying pathophysiology termed inflammation. An environmental insult or local cell damage activates cellular response pathways, including but not limited to p38; local cells then generate cytokines and chemokines, in turn recruiting lymphocytes such as neutrophils and other granulocytes. In a secondary response, the consequences include recruitment of additional lymphocytes such as additional phagocytic cells or cytotoxic T cells, and ultimately the adaptive immune response is initiated through activation of T cells. It is not currently fully understood how this acute inflammatory response becomes a chronic response leading to diseases such as rheumatoid arthritis (RA), atherosclerosis, chronic obstructive pulmonary disease (COPD), inflammatory bowel disease (IBD), etc. Nevertheless, the features of inflammation are recognized to contribute to a large number of chronic diseases and pathways such as the p38 pathway are accepted to contribute to the initiation of inflammatory diseases.

For example, atherosclerosis is regarded as a chronic inflammatory disease, which develops in response to injury of the vessel wall and is characterized by the complex development of an occlusive and prothrombotic atheroma. The pathogenesis of this lesion generally involves endothelial dysfunction (reduced bioavailable NO), adhesion molecule expression, adhesion and infiltration of leukocytes, cytokine and growth factor generation, accumulation of foam cells, expansion of extracellular lipid and matrix, activation of matrix metalloproteases (MMPs) and proliferation of vascular smooth muscle cells.

The discovery of p38 (initially termed CSBP, now p38; the isoforms p38α and p380 are the targets of the compounds described) provided a mechanism of action of a class of anti-inflammatory compounds for which SK&F 86002 was the prototypic example. These compounds inhibited IL-1 and TNF synthesis in human monocytes at concentrations in the low uM range [Lee, et al., Int. J. Immunopharmac. 10(7), 835 (1988)] and exhibited activity in animal models which are refractory to cyclooxygenase inhibitors [Lee; et al., Annals N.Y. Acad. Sci., 696, 149 (1993)].

The mechanism by which stress signals (including bacterial and viral infection, pro-inflammatory cytokines, oxidants, UV light and osmotic stress) activate p38 is through activation of kinases upstream from p38 which in turn phosphorylate p38 at threonine 180 and tyrosine 182 resulting in p38 activation. MAPKAP kinase-2 and MAPKAP kinase-3 have been identified as downstream substrates of CSBP/p38 which in turn phosphorylate heat shock protein Hsp27 and other substrates. Additional downstream substrates known to be phosphorylated by p38 include kinases (Mnk1/2, MSK1/2 and PRAK) and transcription factors (CHOP, MEF2, ATF2 and CREB). While many of the signaling pathways required for transduction of stress stimuli remain unknown it appears clear that many of the substrates for p38 listed above are involved. [Cohen, P. Trends Cell Biol., 353-361 (1997) and Lee, J. C. et al, Pharmacol. Ther. vol. 82, nos. 2-3, pp. 389-397, 1999]. There is also emerging evidence that p38 is involved in modulation of the activity of the NF-kB signalling pathway through a role in histone phosphorylation or acetylation, or through reduction of transcription competence of the NF-kB complex [Saccini, S, Nature Immunol., 3: 69-75, (2002); Carter, A B et al J Biol Chem 274: 30858-63 (1999)]. Finally, a role for p38 in generation of response to IFNs through activation by the Type I IFN receptor has been described [Platanias, Pharmacol. Therap. 98:129-142 (2003)]. Activation of p38 is involved in the transcriptional regulation of IFN sensitive genes through modification of specific transcription factors binding to promoter elements in these genes. Direct phosphorylation of STATs by p38 has not been conclusively demonstrated.

In addition to inhibiting IL-1 and TNF upregulation in response to inflammatory stimuli, p38 kinase inhibitors (e.g., SK&F 86002 and SB-203580) are effective in a number of different cell types in decreasing the synthesis of a wide variety of pro-inflammatory proteins including, IL-6, IL-8, GM-CSF, RANTES and COX-2. Inhibitors of p38 kinase have also been shown to suppress the TNF-induced expression of VCAM-1 on endothelial cells, the TNF-induced phosphorylation and activation of cytosolic PLA2 and the IL-1-stimulated synthesis of collagenase and stromelysin. These and additional data demonstrate that p38 is involved not only cytokine synthesis in response to stress, but also in propagating the consequent cytokine signaling [CSBP/P38 kinase reviewed in Cohen, P. Trends Cell Biol., 353-361 (1997)].

Interleukin-1 (IL-1) and Tumor Necrosis Factor (TNF) are important inflammatory cytokines produced by a variety of cells, such as monocytes, macrophages, and smooth muscle cells. IL-1 has been demonstrated to mediate a variety of biological activities thought to be important in immunoregulation and other physiological conditions such as inflammation [See, e.g., Dinarello et al., Rev. Infect. Disease, 6, 51 (1984)]. The myriad of known biological activities of IL-1 include the activation of T helper cells, induction of fever, stimulation of prostaglandin or collagenase production, neutrophil chemotaxis, induction of acute phase proteins and the suppression of plasma iron levels.

There are many disease states in which excessive or unregulated IL-1 production is implicated in exacerbating and/or causing the disease. These include rheumatoid arthritis, osteoarthritis, endotoxemia and/or toxic shock syndrome, other acute or chronic inflammatory disease states such as the inflammatory reaction induced by endotoxin or inflammatory bowel disease; tuberculosis, atherosclerosis, muscle degeneration, cachexia, psoriatic arthritis, Reiter's syndrome, rheumatoid arthritis, gout, traumatic arthritis, rubella arthritis, and acute synovitis. Evidence also links IL-1 activity to diabetes and pancreatic β cells [review of the biological activities which have been attributed to IL-1 Dinarello, J. Clinical Immunology, 5 (5), 287-297 (1985)].

Excessive or unregulated TNF production has been implicated in mediating or exacerbating a number of diseases including rheumatoid arthritis, rheumatoid spondylitis, osteoarthritis, gouty arthritis and other arthritic conditions; sepsis, septic shock, endotoxic shock, gram negative sepsis, toxic shock syndrome, adult respiratory distress syndrome, cerebral malaria, chronic obstructive pulmonary disease, silicosis, pulmonary sarcoisosis, bone resorption diseases, reperfusion injury, graft vs. host reaction, allograft rejections, fever and myalgias due to infection, such as influenza, cachexia secondary to infection or malignancy, cachexia, secondary to acquired immune deficiency syndrome (AIDS), AIDS, ARC (AIDS related complex), keloid formation, scar tissue formation, Crohn's disease, ulcerative colitis, or pyresis.

Inflammatory diseases are also marked by increases in IL-6 and C-reactive protein (CRP), both of which are sensitive to inhibition by p38 inhibitors. IL-6 stimulation of CRP production is directly inhibited by p38 inhibitors in human vascular endothelial cells, and CRP is produced by hepatocytes in response to IL-6. CRP is considered a major risk factor for cardiovascular disease [Circulation 2003.107: 363-369] and may be a significant independent risk factor for chronic obstructive pulmonary disease [Circulation 2003.107:1514-1519]. IL-6 is also upregulated in endometriosis [Bedaiwy et al., 2002, Human Reproduction 17:426-431; Witz, 2000, Fertility and Sterility 73: 212-214].

Interleukin-8 (IL-8) and RANTES are chemotactic factors produced by several cell types including mononuclear cells, fibroblasts, endothelial cells, epithelial cells, neutrophils and T cells. Chemokine production is induced by pro-inflammatory stimuli such as IL-1, TNF, or lipopolysachharide (LPS), or viral infection. IL-8 stimulates a number of functions in vitro. It has been shown to have chemoattractant properties for neutrophils, T-lymphocytes, and basophils. In addition it induces histamine release from basophils from both normal and atopic individuals as well as lysozomal enzyme release and respiratory burst from neutrophils. IL-8 has also been shown to increase the surface expression of Mac-1 (CD11b/CD18) on neutrophils without de novo protein synthesis, which may contribute to increased adhesion of the neutrophils to vascular endothelial cells. Many diseases are characterized by massive neutrophil infiltration. Conditions such as chronic obstructive pulmonary disease associated with an increase in IL-8 production would benefit by compounds which are suppressive of IL-8 production. RANTES is produced by cells such as epithelial cells and airway smooth muscle in response to infection or cytokine stimulation. Its main chemoattraction is for T cell subtypes and blood-borne monocytes.

IL-1, TNF and other cytokines affect a wide variety of cells and tissues and these cytokines as well as other leukocyte derived cytokines are important as critical inflammatory mediators of a wide variety of disease states and conditions. The inhibition of these cytokines is of benefit in controlling, reducing and alleviating many of these disease states.

In addition to the involvement of p38 signaling in the production of IL-1, TNF, IL-8, IL-6, GM-CSF, COX-2, collagenase and stromelysin, signal transduction via CSBP/p38 is required for the effector functions of several of these same pro-inflammatory proteins plus many others. For example, growth factors such as VEGF, PDGF, NGF signal through surface receptors which in turn activate cellular signaling pathways including p38 MAPK [Ono, K. and Han, J., Cellular Signalling, 12 1-13 (2000); Kyriakis, J M and Avruch, J. Physiol Rev 81: 807-869 (2001)]. TGFχ, a key molecule in the control of inflammatory response, also activates p38 as a consequence of engagement of the TGFβ receptor. The involvement of CSBP/p38 in multiple stress-induced signal transduction pathways provides additional rationale for the potential utility of CSBP/p38 in the treatment of diseases resulting from the excessive and destructive activation of the immune system, or chronic inflammation. This expectation is supported by the potent and diverse activities described for CSBP/p38 kinase inhibitors [Badger, et al., J. Pharm. Exp. Thera. 279 (3): 1453-1461.(1996); Griswold, et al, Pharmacol. Comm. 7, 323-229 (1996); Jackson, et al., J. Pharmacol. Exp. Ther. 284, 687-692 (1998); Underwood, et al., J. Pharmacol. Exp. Ther. 293, 281-288 (2000); Badger, et al., Arthritis Rheum. 43, 175-183 (2000)].

Chronic inflammation is also characterized by ongoing remodeling and repair of affected tissue, leading in some cases to excess fibrotic tissue. A role for p38 MAPK in fibrosis is supported by findings that this enzyme mediates signaling of transforming growth factor beta (TGF-β) on markers and proteins of fibrosis. For example, it has been shown that TGF-β increases the kinase activity of p38 MAPK through the TGF-β activated kinase TAK-1 (Hanafusa et al., 1999, J. Biol. Chem. 274:27161-27167). Furthermore, the p38 inhibitor SB-242235 inhibited the TGF-β-induced increases in fibronectin and thrombospondin (Laping et al., 2002, Molec. Pharmacol. 62:58-64). These results show that p38 MAPK is a key signaling intermediate for the effect of the pro-fibrotic cytokine TGF-β on components of the extracellular matrix and markers of fibrosis.

P38 also plays a role in directing survival and apoptosis of cells in response to various stimuli. Both survival and apoptosis can be p38 regulated depending on the stimulus and the cell type [Morin and Huot, Cancer Research. 64:1893-1898 (2004)]. For example, TGF-beta can stimulate apoptosis in murine hepatocytes through activation of gadd45b, a protein involved in cell-cycle control, in a p38 mediated process [Yoo et al, J. Biol. Chem. 278:43001-43007, (2003)]. In a different response pathway, UV-stress can activate p38 and trigger apoptosis of a damaged cell. P38 has also been shown to promote survival of lymphocytes in response to stress, including neutrophils and CD8+ T cells.

There remains a need for treatment, in this field, for compounds which are cytokine suppressive anti-inflammatory drugs, i.e. compounds which are capable of inhibiting the CSBP/p38/RK kinase. The present invention is directed to such novel compounds which are inhibitors of p38 kinase.

### SUMMARY OF THE INVENTION

This invention relates to the novel compounds of Formula (I), and pharmaceutical compositions comprising a compound of Formula (I), and a pharmaceutically acceptable diluent or carrier.

This invention relates to a method of treating a CSBP/RK/p38 kinase mediated disease in a mammal in need thereof, which comprises administering to said mammal an effective amount of a compound of Formula (I).

This invention relates to a method of inflammation in a mammal in need thereof, which comprises administering to said mammal an effective amount of a compound of Formula (I).

This invention also relates to a method of inhibiting cytokines and the treatment of a cytokine mediated disease, and the inflammation associated therewith, in a mammal in need thereof, which comprises administering to said mammal an effective amount of a compound of Formula (I).

This invention more specifically relates to a method of inhibiting the production of IL-1, IL-8, or TNF in a mammal in need thereof which comprises administering to said mammal an effective amount of a compound of Formula (I).

Accordingly, the present invention provides for a compound of the formula: 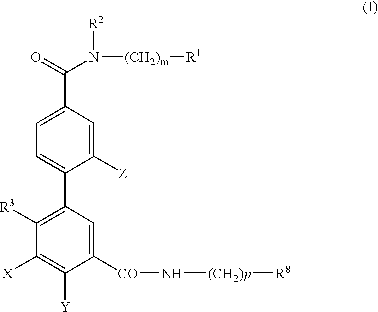
wherein

R¹ is selected from hydrogen, C₁₋₆alkyl optionally substituted by up to three groups independently selected from C₁₋₆alkoxy, halogen and hydroxy, C₃₋₇cycloalkyl optionally substituted independently by one or more C₁₋₆alkyl groups, an aryl, heteroaryl, or heterocyclic ring, all optionally substituted, independently, by up to three groups selected from R⁵ and R⁶;

R² is selected from hydrogen, C₁₋₆alkyl or a —(CH₂)ₚ—C₃₋₇cycloalkyl optionally substituted independently by one or more C₁₋₆alkyl groups,

or the (CH₂)ₘR¹ and R², together with the nitrogen atom to which they are bound, form an optionally substituted, four- to six-membered heterocyclic ring optionally containing at least one additional heteroatom selected from oxygen, nitrogen or sulfur;

R³ is halogen or methyl;

R⁴ is hydrogen, C₁₋₆ alkyl, halo-substituted-C₁₋₄ alkyl, or C₃₋₇cycloalkyl;

R⁵ is selected from C₁₋₆alkyl, OR⁴, —(CH₂)ₚ—C₃₋₇cycloalkyl optionally substituted independently by one or more C₁₋₆alkyl groups, —CONR⁹R¹⁰, —NHCOR¹⁰, —SO₂NHR⁹, —(CH₂)_{q}NHSO₂R¹⁰, halogen, CN, —(CH₂)_{q}NR¹¹R¹², and trifluoromethyl;

R⁶ is selected from hydrogen, C₁₋₆alkyl, OR⁴, halogen, trifluoromethyl and —(CH₂)_{q}NR¹¹R¹²;

R⁸ is selected from hydrogen, C₁₋₆alkyl, C₃₋₇cycloalkyl optionally substituted by one or more C₁₋₆alkyl groups, OH, C₁₋₆alkyl optionally substituted by one or more hydroxyl groups, CONHR⁹, phenyl optionally substituted by R¹³ and/or R¹⁴, or a heteroaryl optionally substituted by R¹³ and/or R¹⁴;

R⁹ and R¹⁰ are each independently selected from hydrogen and C₁₋₆alkyl, or

R⁹ and R¹⁰, together with the nitrogen atom to which they are bound, form a five- to six-membered heterocyclic ring optionally containing one additional heteroatom selected from oxygen, sulfur and N—R¹⁵, wherein the ring is optionally substituted by up to two C₁₋₆alkyl groups;

R¹¹ is selected from hydrogen, C₁₋₆alkyl and —(CH₂)ₚ—C₃₋₇cycloalkyl optionally substituted by one or more C₁₋₆alkyl groups,

R¹² is selected from hydrogen and C₁₋₆alkyl, or

R¹¹ and R¹², together with the nitrogen atom to which they are bound, form a five or six-membered heterocyclic ring optionally containing one additional heteroatom selected from oxygen, sulfur and N—R¹⁵;

R¹³ is selected from C₁₋₆alkyl, C₁₋₆alkoxy, —(CH₂)ₚ—C₃₋₇cycloalkyl optionally substituted by one or more C₁₋₆alkyl groups, —CONR⁹R¹⁰, —NHCOR¹⁰, halogen, CN, —(CH₂)_{q}NR¹¹R¹², trifluoromethyl, phenyl optionally substituted independently by one or more R¹⁴ groups, heterocyclic optionally substituted independently by one or more R¹⁴ groups, and a heteroaryl optionally substituted independently by one or more R¹⁴ groups;

R¹⁴ is selected from C₁₋₆alkyl, C₁₋₆alkoxy, halogen, halo-substituted C₁₋₄ alkyl, and NR¹¹R¹²;

R¹⁵ is selected from hydrogen and methyl;

X and Y are each independently selected from hydrogen, methyl and halogen;

Z is selected from —(CH₂)ₛCOOR¹⁶, or —(CH₂)ₛCONR¹⁶R¹⁷;

R¹⁶ and R¹⁷ are independently selected from hydrogen, optionally substituted C₁₋₆alkyl, —(CR₂₀R₂₁)ᵥOR¹⁸, —(CR₂₀R₂₁)ᵥNR¹⁸R¹⁹, —(CR₂₀R₂₁)ᵥNHSO₂R¹⁸, —(CR₂₀R₂₁)ᵥCONR¹⁸R¹⁹, —(CR₂₀R₂₁)ᵥCOOR¹⁸, optionally substituted —(CR₂₀R₂₁)ₜ heteroaryl, optionally substituted —(CR₂₀R₂₁)ₜaryl, optionally substituted —(CR₂₀R₂₁)ₜ heterocyclic, optionally substituted —(CR₂₀R₂₁)ₜ C3-7cycloalkyl, or optionally substituted —(CR₂₀R₂₁)ₜC3-7cycloalkenyl; or

R¹⁶ and R¹⁷, together with the nitrogen atom to which they are bound, form an optionally substituted five- to six-membered ring optionally containing at least one additional heteroatom selected from oxygen, sulfur and N—R¹⁵;

R¹⁸ and R¹⁹ are each independently selected from hydrogen and C₁₋₆alkyl optionally substituted by up to two hydroxy groups; or

R¹⁸ and R¹⁹, together with the nitrogen atom to which they are bound, form a five- to six-membered ring, optionally containing one additional heteroatom selected from oxygen, sulfur and N—R¹⁵, and wherein the ring is optionally substituted by up to two groups independently selected from oxo, halogen and C₁₋₆alkyl;

R₂₀ and R₂₁ are independently selected from hydrogen or C₁₋₄ alkyl;

m is 0 or an integer selected from 1, 2, 3 and 4;

p is 0 or an integer selected from 1 and 2;

q is 0 or an integer selected from 1, 2 and 3;

r is 0 or an integer of 1;

s is 0 or an integer selected from 1, 2, 3 and 4; and

t is 0 or an integer selected from 1, 2, 3, 4, 5 and 6;

v is an integer selected from 1, 2, 3, 4, 5, and 6;

or a pharmaceutically acceptable salt or derivative thereof.

### DETAILED DESCRIPTION OF THE INVENTION

The novel compound of Formula (I) are discussed in greater detail described below.

Suitably, R¹ is selected from hydrogen; a C₁₋₆alkyl optionally substituted by up to three groups independently selected from C₁₋₆alkoxy, halogen, hydroxy, and NR¹¹R¹²; a C₃₋₇cycloalkyl optionally substituted by one or more C₁₋₆alkyl groups; an aryl; a heteroaryl; or a heterocyclic ring, and wherein the aryl, heteroaryl, and heterocyclic rings are all optionally substituted, independently, by up to three groups selected from R⁵ and R⁶.

A representative example of R¹ as a C₃₋₆cycloalkyl is an optionally substituted cyclopropyl, or cyclohexylring. A representative example of R¹ as a C₁₋₆alkyl, includes but is not limited to 2-methylpropyl, 1,2-dimethylpropyl, 2,2-dimethylpropyl, or a 1,2,2-trimethylpropyl group.

Representative examples of R¹ as an optionally substituted heteroaryl group include but are not limited to a thiazole ring, diazole, imidazole, or thiadiazole ring.

Representative examples of R¹ as an optionally substituted heterocyclic ring include but are not limited to a piperazine ring, a piperidine, or a pyrrolidinyl ring.

In one embodiment R¹ is a phenyl ring optionally substituted R⁵ or R⁶.

In another embodiment, R¹ is selected from C₁₋₆alkyl, C₃₋₇cycloalkyl or a phenyl optionally substituted by up to three groups selected from R⁵ and R⁶.

In another embodiment R¹ is selected from an optionally substituted heteroaryl ring. In another embodiment, R¹ is an optionally substituted thiazole ring, pyrazole, diazole, imidazole, or thiadiazole ring.

Suitably, R² is selected from hydrogen, C₁₋₆alkyl and (CH₂)ₚ—C₃₋₇cycloalkyl optionally substituted by one or more C₁₋₆alkyl groups or (CH₂)ₘR¹ and R² together with the nitrogen atom to which they are bound, form an optionally substituted four- to six-membered heterocyclic ring, which may also optionally contain at least one additional heteroatom selected from oxygen, nitrogen and sulfur. This heterocyclic ring may be optionally substituted by up to three substituents, independently selected from C₁₋₆alkyl, halogen, hydroxyl, C₁₋₄ alkoxy, C₁₋₄ alkylthio, C₁₋₄alkylsulfinyl, CH₂OR⁴, amino, mono and di-C₁₋₆ alkyl substituted amino. If the heterocyclic ring contains an additional nitrogen, the nitrogen atom itself may also be optionally substituted by an oxide, or a C₁₋₆alkyl group.

In one embodiment the (CH₂)ₘR¹ and R² cyclized ring may be an optionally substituted piperidine, piperazine, or pyrollidine ring, such as a 4-methyl-1-piperazinyl ring.

In one embodiment R² is hydrogen, or a branched or linear C₁₋₅alkyl moiety, such as ethyl, such as methyl, ethyl, n-propyl, isopropyl, n-butyl. Or t-butyl.

In one embodiment of the invention, R² is selected from hydrogen, C₁₋₄alkyl and —CH₂—C₃₋₆cycloalkyl. In another embodiment, R² is hydrogen, ethyl or n-butyl. In another embodiment, R² is hydrogen.

Suitably, R³ is halogen, or methyl.

Suitably, R⁴ is hydrogen, C₁₋₆ alkyl, halo-substituted-C₁₋₄ alkyl, or C₃₋₇ cycloalkyl.

Suitably, R⁵ is selected from C₁₋₆alkyl, C₁₋₆alkoxy, —(CH₂)ₚ—C₃₋₇cycloalkyl optionally substituted by one or more C₁₋₆alkyl groups, —CONR⁹R¹⁰, —NHCOR¹⁰, —SO₂NHR⁹, —(CH₂)_{q}NHSO₂R¹⁰, halogen, CN, OH, —(CH₂)_{q}NR¹¹R¹², and trifluoromethyl.

In one embodiment R⁵ is selected from C₁₋₄alkyl, C₁₋₄alkoxy, —(CH₂)_{q}NHSO₂R¹⁰, halogen, —(CH₂)_{q}NR¹¹R¹² and trifluoromethyl. In another embodiment R⁵ is a C₁₋₄alkyl, such as methyl, or is a C₁₋₄alkoxy, such as methoxy.

Suitably, R⁶ is selected from C₁₋₆alkyl, C₁₋₆alkoxy, halogen, trifluoromethyl and —(CH₂)_{q}NR¹¹R¹². In one embodiment, R⁶ is selected from C₁₋₄alkyl, C₁₋₄alkoxy, halogen and trifluoromethyl. In another embodiment, R⁶ is a C₁₋₄alkyl, such as methyl, or is a C₁₋₄alkoxy, such as methoxy.

Suitably, R⁸ is selected from hydrogen, C₁₋₆alkyl, C₃₋₇cycloalkyl optionally substituted by one or more C₁₋₆alkyl groups, OH, a C₁₋₆alkyl optionally substituted by one or more hydroxyl groups, CONHR⁹, phenyl optionally substituted by R¹³ and/or R¹⁴, or a heteroaryl optionally substituted by R¹³ and/or R¹⁴.

Suitably, when R⁸ is a heteroaryl ring optionally substituted by R¹³ and/or R¹⁴ the ring includes, but is not limited to, a furyl, thienyl, pyrrolyl, oxazolyl, thiazolyl, isoxazolyl, isothiazolyl, imidazolyl, pyrazolyl, oxadiazolyl, triazolyl, tetrazolyl, thiadiazolyl, pyridyl, pyridazinyl, pyrimidinyl, pyrazinyl or triazinyl ring, or as further defined herein.

In one embodiment when R⁸ is a heteroaryl optionally substituted R¹³ and/or R¹⁴ the heteroaryl ring is suitably a pyrazole, 1,2,4-thiadiazole, 1,3-thiazole, isoxazole, isothiazole, oxadiazolyl, or a pyridine.

In one embodiment, R⁸ is selected from hydrogen, C₃₋₇cycloalkyl optionally substituted by one or more C₁₋₆alkyl groups, CONHR⁹, phenyl optionally substituted by R¹³ and/or R¹⁴, and heteroaryl optionally substituted by R¹³ and/or R¹⁴. In another embodiment, R⁸ is selected from C₃₋₇cycloalkyl, CONHR⁹, phenyl optionally substituted by R¹³ and/or R¹⁴ and heteroaryl optionally substituted by R¹³ and/or R¹⁴. In one embodiment when R⁸ is C₃₋₆cycloalkyl, p=0. A representative example of R⁸ is C₃₋₆cycloalkyl, such as cyclopropyl.

In one embodiment R⁸ is C₁₋₆alkyl, OH, or a C₁₋₆alkyl optionally substituted by one or more hydroxyl groups.

Suitably, R⁹ and R¹⁰ are each independently selected from hydrogen and C₁₋₄alkyl, or R⁹ and R¹⁰, together with the nitrogen atom to which they are bound, form a five- to six-membered heterocyclic ring optionally containing one additional heteroatom selected from oxygen, sulfur and N—R¹⁵, wherein the ring is optionally substituted by up to two C₁₋₆alkyl groups.

Suitably, R¹¹ is selected from hydrogen, C₁₋₆alkyl and —(CH₂)ₚ—C₃₋₇cycloalkyl optionally substituted by one or more C₁₋₆alkyl groups.

Suitably, R¹² is selected from hydrogen and C₁₋₆alkyl, or R¹¹ and R¹², together with the nitrogen atom to which they are bound, form a five or six-membered heterocyclic ring optionally further containing one additional heteroatom N—R¹⁵.

Suitably, R¹³ is selected from hydrogen, C₁₋₆alkyl, C₁₋₆alkoxy, —(CH₂)ₚ—C₃₋₇cycloalkyl optionally substituted by one or more C₁₋₆alkyl groups, —CONR⁹R¹⁰, —NHCOR¹⁰, halogen, CN, —(CH₂)_{q}NR¹¹R¹², trifluoromethyl, a phenyl ring optionally substituted by one or more R¹⁴ groups, heterocyclic optionally substituted independently by one or more R¹⁴ groups, and a heteroaryl ring optionally substituted by one or more R¹⁴ groups.

Suitably, R¹⁴ is selected from hydrogen, C₁₋₆alkyl, C₁₋₆alkoxy, halogen, halo-substituted C₁₋₄ alkyl, such as trifluoromethyl, and NR¹¹R¹².

In one embodiment, R¹³ is selected from C₁₋₄alkyl, C₁₋₄alkoxy, halogen, —(CH₂)_{q}NR¹¹R¹², a phenyl ring optionally substituted by one or more R¹⁴ groups, or a heteroaryl ring optionally substituted by one or more R¹⁴ groups.

In one embodiment, R¹⁴ is selected from C₁₋₄alkyl, C₁₋₄alkoxy and —NR¹¹R¹².

In one embodiment, R¹³ and R¹⁴ are independently selected from hydrogen or C₁₋₄alkyl.

Suitably, R¹⁵ is hydrogen, or methyl.

Suitably, X and Y are each independently selected from hydrogen, methyl and halogen. In one embodiment, X and Y are each independently selected from hydrogen, chlorine and fluorine. A representative example of halogen is fluorine. A further representative example of X is hydrogen. A representative example of Y is hydrogen.

Suitably, Z is selected from —(CH₂)ₛCOOR¹⁶, or —(CH₂)ₛCONR¹⁶R¹⁷. In one embodiment Z is —(CH₂)ₛCONR¹⁶R¹⁷.

Suitably, R¹⁶ and R¹⁷ are independently selected from hydrogen, optionally substituted C₁₋₆alkyl, —(CR₂₀R₂₁)ᵥOR¹⁸, —(CR₂₀R₂₁)ᵥNR¹⁸R¹⁹, —(CR₂₀R₂₁)ᵥNHSO₂R¹⁸, —(CR₂₀R₂₁)ᵥCONR¹⁸R¹⁹, —(CR₂₀R₂₁)ᵥCOOR¹⁸, optionally substituted —(CR₂₀R₂₁)ₜheteroaryl, optionally substituted —(CR₂₀R₂₁)ₜaryl, optionally substituted —(CR₂₀R₂₁)ₜheterocyclic, optionally substituted —(CR₂₀R₂₁)ₜ C₃₋₇cycloalkyl, optionally substituted —(CR₂₀R₂₁)ₜC₄₋₇cycloalkenyl, or R¹⁶ and R¹⁷ together with the nitrogen atom to which they are bound, form an optionally substituted five- to six-membered heterocyclic ring optionally containing at least one additional heteroatom selected from oxygen, sulfur and N—R¹⁵.

Suitable, v is an integer selected from 1, 2, 3, 4, 5, and 6.

In one embodiment R¹⁶ is hydrogen, C₁₋₆alkyl, C₁₋₆alkyl optionally substituted one or more times independently by hydroxyl, halogen, C₁₋₆alkoxy, and NR₇R_{7′}, wherein R₇ and R_{7′} are each independently hydrogen or C₁₋₄ alkyl.

In another embodiment R¹⁶ is propyl, isopropyl, 2-hydroxypropyl, 3-hydroxypropyl, 2,2,2-trifluoroethyl, dimethylamino)ethyl, hydrogen, 3-(ethyloxy)propyl, 5-hydroxypentyl, (dibutylamino)propyl, or 1-(methylethyl)oxy)propyl.

Suitably, the R¹⁶ moiety when it is a C₁₋₆alkyl (branched or linear) is optionally substituted independently, one or more times by halogen, such as fluorine, chlorine, bromine or iodine; hydroxy; C₁₋₆ alkoxy, such as methoxy or ethoxy; halosubstituted C₁₋₆alkoxy; S(O)m′ alkyl, such as methyl thio, methylsulfinyl or methyl sulfonyl, wherein m′ is 0, 1 or 2; —C(O); NR₇R_{7′}, wherein R₇ and R_{7′} are each independently hydrogen or C₁₋₄ alkyl, such as amino or mono or -disubstituted C₁₋₄ alkyl, or wherein the R₇ and R_{7′} together with the nitrogen to which they are attached can cyclize to form a 5 to 7 membered ring, which ring optionally contains an additional heteroatom selected from O/N/S; C₁₋₆ alkyl, C₃₋₇cycloalkyl, or C₃₋₇cycloalkyl C₁₋₆ alkyl group, such as methyl, ethyl, propyl, isopropyl, t-butyl, etc. or cyclopropyl methyl; halosubstituted C₁₋₆ alkyl, such CF₂CF₂H, or CF₃; cyano; an optionally substituted aryl, such as phenyl, or an optionally substituted arylalkyl, such as benzyl or phenethyl, wherein these aryl containing moieties may also be substituted one to two times by halogen; hydroxy; hydroxy substituted alkyl; C₁₋₆ alkoxy; S(O)_{m′}alkyl; amino, mono & di-substituted C₁₋₄ alkyl amino; C₁₋₄ alkyl, or CF₃. In one embodiment the C₁₋₆alkyl is optionally substituted by one or two hydroxy groups.

When R¹⁶ is an optionally substituted —(CR₂₀R₂₁)ₜheteroaryl ring, optionally substituted —(CR₂₀R₂₁)ₜaryl ring, optionally substituted —(CR₂₀R₂₁)ₜheterocyclic ring, optionally substituted —(CR₂₀R₂₁)ₜC₃₋₇cycloalkyl ring or an optionally substituted —(CR₂₀R₂₁)ₜC₃₋₇ cycloalkenyl ring, the rings may be substituted independently, one or more times, suitably 1 to 3 times, by halogen, such as fluorine, chlorine, bromine or iodine; hydroxy; hydroxy substituted C₁₋₆alkyl; C₁₋₆ alkoxy, such as methoxy or ethoxy; halosubstituted C₁₋₆ alkoxy; S(O)_{m′}C₁₋₆ alkyl, such as methyl thio, methylsulfinyl or methyl sulfonyl, wherein m′ is 0, 1 or 2; —C(O); NR₇R_{7′}, wherein R₇ and R_{7′} are each independently hydrogen or C₁₋₄ alkyl, such as amino or mono or -disubstituted C₁₋₄ alkyl or wherein the R₇ and R_{7′} can together with the nitrogen to which they are attached cyclize to form a 5 to 7 membered ring which optionally contains an additional heteroatom selected from O/N/S; S(O)₂NR₇R_{7′}; C₁₋₆ alkyl, C₃₋₇cycloalkyl, or C₃₋₇cycloalkyl C₁₋₆ alkyl group, such as methyl, ethyl, propyl, isopropyl, t-butyl, etc. or cyclopropyl methyl; halosubstituted C₁₋₆ alkyl, such CF₂CF₂H, or CF₃; an optionally substituted aryl, such as phenyl, or an optionally substituted arylalkyl, such as benzyl or phenethyl, wherein these aryl containing moieties may also be substituted one to two times by halogen; hydroxy; hydroxy substituted alkyl; C₁₋₆ alkoxy; S(O)_{m′}alkyl; amino, mono & di-substituted C₁₋₄ alkyl amino; C₁₋₄ alkyl, or CF₃. In one embodiment, the rings are substituted by one or two groups independently selected from halogen, C₁₋₆alkyl and oxo, and C₁₋₆alkoxy. It is recognized that when R¹⁶ is an optionally substituted —(CH₂)ₜheteroaryl ring, or optionally substituted —(CH₂)ₜheterocyclic ring, and the heteroaryl or the heterocyclic ring contains a nitrogen, the nitrogen itself may also be directly substituted, as in 1-ethyl-pyrrolidinyl ring.

Suitably, when R¹⁶ is an optionally substituted —(CR₂₀R₂₁)ₜheteroaryl ring, the ring includes, but is not limited to, a furyl, thienyl, pyrrolyl, oxazolyl, thiazolyl, isoxazolyl, isothiazolyl, imidazolyl, pyrazolyl, oxadiazolyl, triazolyl, tetrazolyl, thiadiazolyl, pyridyl, pyridazinyl, pyrimidinyl, pyrazinyl, triazinyl ring, indolyl, isoindolyl, azaindolyl, benzoxazolyl, benzimidazolyl, benzothiazolyl, benzofuranyl, benzothiophenyl, quinolyl, isoquinolyl, quinazolinyl, cinnolinyl or phthalazinyl ring, or as further defined herein.

Suitably, when R¹⁶ is an optionally substituted —(CR₂₀R₂₁)ₜheterocyclic ring, the ring includes, but is not limited to, aziridinyl, pyrrolinyl, pyrrolidinyl, imidazolinyl, imidazolidinyl, pyrazolinyl, pyrazolidinyl, piperidyl, piperazinyl, morpholino and thiomorpholino rings, or as further defined herein.

When R¹⁶ and R¹⁷ together with the nitrogen atom to which they are bound form an optionally substituted five- to six-membered heterocyclic ring which ring may optionally containing at least one additional heteroatom selected from oxygen, sulfur and N—R¹⁵ the cyclized ring may be optionally substituted, independently, one or more times by halogen, such as fluorine, chlorine, bromine or iodine; hydroxy; C₁₋₆ alkoxy, such as methoxy or ethoxy; halosubstituted C₁₋₆alkoxy; S(O)m′ alkyl, such as methyl thio, methylsulfinyl or methyl sulfonyl, wherein m′ is 0, 1 or 2; —C(O); NR₇R_{7′}, wherein R₇ and R_{7′} are each independently hydrogen or C₁₋₄ alkyl, such as amino or mono or -disubstituted C₁₋₄ alkyl or wherein the R₇ and R_{7′} can together with the nitrogen to which they are attached cyclize to form a 5 to 7 membered ring which optionally contains an additional heteroatom selected from O/N/S, such as a pyrrolidinyl ring; C₁₋₆ alkyl, C₃₋₇cycloalkyl, or C₃₋₇cycloalkyl C₁₋₆ alkyl group, such as methyl, ethyl, propyl, isopropyl, t-butyl, etc. or cyclopropyl methyl; halosubstituted C₁₋₆ alkyl, such CF₂CF₂H, or CF₃; an optionally substituted aryl, such as phenyl, or an optionally substituted arylC₁₋₄alkyl, such as benzyl or phenethyl, and wherein the aryl and arylalkyl moieties may also be substituted independently, one or two times by halogen; hydroxy; hydroxy substituted alkyl; C₁₋₆ alkoxy; S(O)_{m′}alkyl; amino, mono & di-substituted C₁₋₄alkyl amino; C₁₋₄ alkyl, or CF₃. In one embodiment the cyclized ring is a piperidine ring or a piperazinyl ring. In another embodiment the cyclized ring is optionally substituted by C₁₋₆ alkyl, hydroxy or C(O). In one embodiment R¹⁶ and R¹⁷ together form an optionally substituted piperidinyl or piperazinyl ring.

In one embodiment when R¹⁶ is an optionally substituted —(CR₂₀R₂₁)ₜheteroaryl ring, the heteroaryl ring is an optionally substituted pyridine, thiazole, pyrrole, diazole, imidazole, or a thiadiazole ring. In another embodiment R¹⁶ is a thiazole ring.

In one embodiment when R¹⁶ is an optionally substituted —(CR₂₀R₂₁)ₜheterocyclic ring, the heterocyclic ring is a piperazine ring, a piperidine, or a pyrrolidinyl ring.

In one embodiment when R¹⁶ is an optionally substituted —(CR₂₀R₂₁)ₜaryl ring, the aryl ring is an optionally substituted phenyl. In another embodiment the phenyl is optionally substituted independently, 1 to 3 times by C₁₋₆ alkyl, or S(O)₂NR₇R_{7′}.

In one embodiment R¹⁶ is an optionally substituted —(CR₂₀R₂₁)ₜ C₃₋₇cycloalkyl. In another embodiment R¹⁶ is cyclopropyl, cyclopentyl, or a cyclohexylC1-6 alkyl.

In one embodiment, R¹⁶ is selected from hydrogen, optionally substituted C₁₋₆alkyl; —(CR₂₀R₂₁)ᵥOR¹⁸, —(CR₂₀R₂₁)ᵥNR¹⁸R¹⁹, —(CR₂₀R₂₁)ᵥCOOR¹⁸, —(CR₂₀R₂₁)ₜheteroaryl, —(CR₂₀R₂₁)ₜheterocyclic, and —(CR₂₀R₂₁)ₜaryl. In another embodiment, the heteroaryl, heterocyclic, and aryl moieties are optionally substituted, independently 1 to 3 times by halogen, C₁₋₆alkyl, and C₁₋₆alkoxy.

In one embodiment R¹⁶ is an optionally substituted (CR₂₀R₂₁)ₜheteroaryl, optionally substituted —(CR₂₀R₂₁)ₜ aryl, or an optionally substituted —(CR₂₀R₂₁)ₜheterocyclic. In one embodiment R¹⁶ is an optionally substituted thiazolyl, optionally substituted phenyl, optionally substituted pyridine, optionally substituted imidazole, optionally substituted piperidinyl, optionally substituted piperazinyl, optionally substituted phenylC₁₋₆alkyl, or optionally substituted pyrrolidinyl C₁₋₆alkyl. In another embodiment R¹⁶ is 1,3-thiazolyl, optionally substituted phenyl, pyridine, imidazole, piperidinyl, piperazinyl, benzyl, phenylbutyl, phenylethyl, pyrrolidinylethyl, pyrrolidinylmethyl, or (4-methylphenyl)methyl, or (1-ethyl-2-pyrrolidinyl)methyl. In another embodiment R¹⁶ is 1,3-thiazolyl, and t is 0.

One representative example of R¹⁶ is —(CR₂₀R₂₁)ᵥNR¹⁸R¹⁹. Other representative examples of R¹⁶ include hydrogen; C₁₋₆alkyl optionally substituted by up to two hydroxy groups, in particular methyl, ethyl, n-propyl, 2-hydroxypropyl, 2,3-dihydroxypropyl, 4-hydroxybutyl and 2,2-dimethylpropyl; (CR₂₀R₂₁)ᵥOR¹⁸; (CR₂₀R₂₁)ᵥNR¹⁸R¹⁹; (CR₂₀R₂₁)ᵥNHSO₂R¹⁸; (CR₂₀R₂₁)ᵥCONR¹⁸R¹⁹; (CR₂₀R₂₁)ᵥCOOR¹⁸; and a (CR₂₀R₂₁)ₜ heteroaryl ring optionally substituted by up to two groups independently selected from halogen, C₁₋₆alkyl and oxo. In one embodiment the heteroaryl ring is a 5-membered ring containing up to three heteroatoms selected from oxygen, nitrogen and sulphur.

Suitably, R¹⁸ and R¹⁹ are each independently selected from hydrogen and a C₁₋₆alkyl group which is optionally substituted by up to two hydroxy groups, or R¹⁸ and R¹⁹, together with the nitrogen atom to which they are bound, form a five- to six-membered heterocyclic ring optionally containing one additional heteroatom selected from oxygen, sulfur and N—R¹⁵, wherein the ring is optionally substituted by up to two groups independently selected from oxo, halogen and C₁₋₆alkyl.

In one embodiment, R¹⁸ and R¹⁹ are each independently selected from hydrogen and C₁₋₄alkyl. In another embodiment, R¹⁸ and R¹⁹ are each independently selected from hydrogen, methyl, ethyl, 2-hydroxyethyl and isopropyl. A representative example of R¹⁸ and R¹⁹ is methyl. Further representative examples of R¹⁸ and R¹⁹ include hydrogen, ethyl, 2-hydroxyethyl and isopropyl.

In a further embodiment, R¹⁸ and R¹⁹, together with the nitrogen atom to which they are bound, form a five- to six-membered heterocyclic ring optionally containing oxygen, for example pyrrolidinyl or morpholinyl.

Suitably, R₂₀ and R₂₁ are independently selected from hydrogen or C₁₋₄ alkyl.

Suitably, m is 0 or an integer selected from 1, 2, 3 and 4, wherein each carbon atom of the resulting carbon chain may be optionally substituted with up to two groups independently selected from C₁₋₆alkyl and halogen. In one embodiment, m is selected from 0, 1 and 2. In another embodiment, m is selected from 0 and 1. A representative example of m is 1. A further representative example of m is 0.

Suitably, p is 0 or an integer selected from 1 and 2. In one embodiment, p is selected from 0 and 1. A representative example of p is 0.

Suitably, q is 0 or an integer selected from 1, 2 and 3. In one embodiment, q is selected from 0 and 1.

Suitably, r is 0 or an integer of 1. In one embodiment, r is 0.

Suitably, s is 0 or an integer selected from 1, 2, 3 and 4. In one embodiment, s is selected from 0 and 1. A representative example of s is 0. A further representative example of s is 1.

Suitably, t is 0 or an integer selected from 1, 2, 3, 4, 5 and 6. In one embodiment t is selected from 0, 1, 2, 3 and 4. In another embodiment, t is 2, 3, and 4. In another embodiment, t is selected from 2 and 3. A representative example of t is 0, or 2. Further representative examples of t include 1, 3 and 4.

In one embodiment Z is (CH₂)ₛCONR¹⁶R¹⁷, R¹⁶ is 1,3-thiazolyl, t is 0, R⁸ is cyclopropyl, p=0. In a further embodiment R² is hydrogen, R¹ is C₁₋₆alkyl, or C₃₋₇cycloalkyl.

Another aspect of the invention are compounds represented by the formula: 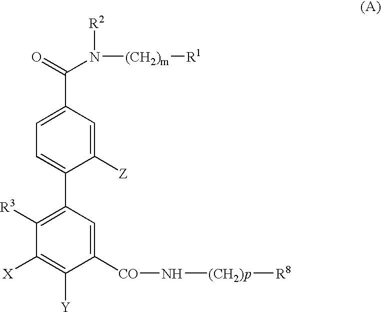
wherein

R¹ is selected from hydrogen; C₁₋₆alkyl optionally substituted by up to three groups independently selected from C₁₋₆alkoxy, halogen and hydroxy; C₃₋₇cycloalkyl optionally substituted independently by one or more C₁₋₆alkyl groups; an aryl, heteroaryl, or heterocyclic ring each optionally substituted, independently, by up to three groups selected from R⁵ and R⁶;

R² is hydrogen, C₁₋₆alkyl or a —(CH₂)ₚ—C₃₋₇cycloalkyl optionally substituted independently by one or more C₁₋₆alkyl groups,

or the (CH₂)ₘR¹ and R², together with the nitrogen atom to which they are bound, form an optionally substituted, four- to six-membered heterocyclic ring optionally containing another heteroatom selected from O/N/S;

R³ is halogen or methyl;

R⁴ is hydrogen, C₁₋₆ alkyl, halo-substituted-C₁₋₄ alkyl, or C₃₋₇cycloalkyl;

R⁵ is independently C₁₋₆alkyl, OR⁴, —(CH₂)ₚ—C₃₋₇cycloalkyl optionally substituted independently by one or more C₁₋₆alkyl groups, —CONR⁹R¹⁰, —NHCOR¹⁰, —SO₂NHR⁹, —(CH₂)_{q}NHSO₂R¹⁰, halogen, CN, —(CH₂)_{q}NR¹¹R¹², and trifluoromethyl;

R⁶ is independently hydrogen, C₁₋₆alkyl, OR⁴, halogen, trifluoromethyl and —(CH₂)_{q}NR¹¹R¹²;

R⁸ is selected from hydrogen, C₁₋₆alkyl, C₃₋₇cycloalkyl optionally substituted by one or more C₁₋₆alkyl groups, OH, a C₁₋₆alkyl optionally substituted by one or more hydroxyl groups, CONHR⁹, phenyl optionally substituted by R¹³ and/or R¹⁴, or a heteroaryl optionally substituted by R¹³ and/or R¹⁴;

R⁹ and R¹⁰ are each independently selected from hydrogen and C₁₋₆alkyl, or

R⁹ and R¹⁰, together with the nitrogen atom to which they are bound, form a five- to six-membered heterocyclic ring optionally containing one additional heteroatom selected from oxygen, sulfur and N—R¹⁵, wherein the ring is optionally substituted by up to two C₁₋₆alkyl groups;

R¹¹ is selected from hydrogen, C₁₋₆alkyl and —(CH₂)ₚ—C₃₋₇cycloalkyl optionally substituted by one or more C₁₋₆alkyl groups,

R¹² is selected from hydrogen and C₁₋₆alkyl, or

R¹¹ and R¹², together with the nitrogen atom to which they are bound, form a five or six-membered heterocyclic ring optionally containing one additional heteroatom selected from oxygen, sulfur and N—R¹⁵;

R¹³ is selected from C₁₋₆alkyl, C₁₋₆alkoxy, —(CH₂)ₚ—C₃₋₇cycloalkyl optionally substituted by one or more C₁₋₆alkyl groups, —CONR⁹R¹⁰, —NHCOR¹⁰, halogen, CN, —(CH₂)_{q}NR¹¹R¹², trifluoromethyl, phenyl optionally substituted independently by one or more R¹⁴ groups, heterocyclic optionally substituted independently by one or more R¹⁴ groups, and a heteroaryl optionally substituted independently by one or more R¹⁴ groups;

R¹⁴ is selected from C₁₋₆alkyl, C₁₋₆alkoxy, halogen, halo-substituted C₁₋₄ alkyl, and NR¹¹R¹²;

R¹⁵ is selected from hydrogen and methyl;

X and Y are each independently selected from hydrogen, methyl and halogen;

Z is selected from —(CH₂)ₛNH₂, or (CH₂)ₛN(R²²)CONR²³R²⁴;

R²³ and R²⁴ are independently selected from hydrogen, optionally substituted C₁₋₆alkyl, —(CR₂₀R₂₁)ᵥOR²⁵, —(CR₂₀R₂₁)ᵥNR²⁵R²⁶, —(CR₂₀R₂₁)ᵥNHSO₂R²⁵, —(CR₂₀R₂₁)ᵥCONR²⁵R²⁶, —(CR₂₀R₂₁)ᵥCOOR²⁵, optionally substituted —(CR₂₀R₂₁)ₜheteroaryl, optionally substituted —(CR₂₀R₂₁)ₜ aryl, optionally substituted —(CR₂₀R₂₁)ₜheterocyclic, optionally substituted —(CR₂₀R₂₁)ₜ C₃₋₇cycloalkyl, or optionally substituted —(CR₂₀R₂₁)ₜ C₃₋₇cycloalkenyl; or

R²³ and R²⁵, together with the nitrogen atom to which they are bound, form an optionally substituted five- to six-membered ring optionally containing at least one additional heteroatom selected from oxygen, sulfur and N—R¹⁵;

R²⁵ and R²⁶ are each independently selected from hydrogen and C₁₋₆alkyl optionally substituted by up to two hydroxy groups; or

R²⁵ and R²⁶, together with the nitrogen atom to which they are bound, form a five- to six-membered ring, optionally containing one additional heteroatom selected from oxygen, sulfur and N—R¹⁵, and wherein the ring is optionally substituted by up to two groups independently selected from oxo, halogen and C₁₋₆alkyl;

R₂₀ and R₂₁ are independently selected from hydrogen or C₁₋₄ alkyl;

R²² is selected from hydrogen or C₁₋₄ alkyl;

m is 0 or an integer selected from 1, 2, 3 and 4;

p is 0 or an integer selected from 1 and 2;

q is 0 or an integer selected from 1, 2 and 3;

r is 0 or an integer of 1;

s is 0 or an integer selected from 1, 2, 3 and 4; and

t is 0 or an integer selected from 1, 2, 3, 4, 5 and 6;

v is an integer selected from 1, 2, 3, 4, 5 and 6;

or a pharmaceutically acceptable salt or derivative thereof.

Compounds of Formula (A) differ from compounds of Formula (I) in their definition of the Z substituent. All remaining terms, for example R¹, R², etc. have the same definitions and substitutions, etc. as indicated herein for Formula (I).

Suitably, Z is —(CH₂)ₛNH₂, or (CH₂)ₛN(R²²)CONR²³R²⁴. In one embodiment Z is (CH₂)ₛN(R²²)CONR²³R²⁴.

Suitably, R²³ and R²⁴ are independently selected from hydrogen, optionally substituted C₁₋₆alkyl, —(CR₂₀R₂₁)ᵥOR²⁵, —(CR₂₀R₂₁)ᵥNR²⁵R²⁶, —(CR₂₀R₂₁)ᵥNHSO₂R²⁵, —(CR₂₀R₂₁)ᵥCONR²⁵R²⁶, —(CR₂₀R₂₁)ᵥCOOR²⁵, optionally substituted —(CR₂₀R₂₁)ₜheteroaryl, optionally substituted —(CR₂₀R₂₁)ₜ aryl, optionally substituted —(CR₂₀R₂₁)ₜheterocyclic, optionally substituted —(CR₂₀R₂₁)ₜ C₃₋₇cycloalkyl, or optionally substituted —(CR₂₀R₂₁)ₜ C₃₋₇cycloalkenyl; or R²³ and R²⁴, together with the nitrogen atom to which they are bound, form an optionally substituted five- to six-membered ring optionally containing at least one additional heteroatom selected from oxygen, sulfur and N—R¹⁵.

Suitably, R²⁵ and R²⁶ are each independently selected from hydrogen and C₁₋₆alkyl optionally substituted by up to two hydroxy groups; or R²⁵ and R²⁶, together with the nitrogen atom to which they are bound, form a five- to six-membered ring, optionally containing one additional heteroatom selected from oxygen, sulfur and N—R¹⁵, and wherein the ring is optionally substituted by up to two groups independently selected from oxo, halogen and C₁₋₆alkyl.

Suitably, R₂₀ and R₂₁ are independently selected from hydrogen or C₁₋₄ alkyl.

Suitably, v is an integer selected from 1, 2, 3, 4, 5 and 6.

Suitably, R²² is selected from hydrogen or C₁₋₄ alkyl.

This invention therefore also relates to the novel compounds of Formula (A), and pharmaceutical compositions comprising a compound of Formula (A), and a pharmaceutically acceptable diluent or carrier.

This invention relates to a method of treating a CSBP/RK/p38 kinase mediated disease, and the inflammation associated therewith, in a mammal in need thereof, which comprises administering to said mammal an effective amount of a compound of Formula (A).

This invention also relates to a method of inhibiting cytokines and the treatment of a cytokine mediated disease, and the inflammation associated therewith, in a mammal in need thereof, which comprises administering to said mammal an effective amount of a compound of Formula (A).

This invention more specifically relates to a method of inhibiting the production of IL-1, IL-8, or TNF in a mammal in need thereof which comprises administering to said mammal an effective amount of a compound of Formula (A).

It is to be understood that the present invention covers all combinations of the representative groups described hereinabove. It is also to be understood that the present invention encompasses compounds of formula (I) in which a particular group or parameter, for example R⁵, R⁶, R⁹, R¹⁰, R¹¹, R¹², R¹⁵, p or q may occur more than once. In such compounds it will be appreciated that each group or parameter is independently selected from the values listed.

Particular compounds according to the invention include those mentioned in the examples and their pharmaceutically derivatives.

As used herein, the term “pharmaceutically acceptable” means a compound which is suitable for pharmaceutical use. Salts and solvates of compounds of the invention which are suitable for use in medicine are those wherein the counterion or associated solvent is pharmaceutically acceptable. However, salts and solvates having non-pharmaceutically acceptable counterions or associated solvents are within the scope of the present invention, for example, for use as intermediates in the preparation of other compounds of the invention and their pharmaceutically acceptable salts and solvates.

As used herein, the term “pharmaceutically acceptable derivative”, means any pharmaceutically acceptable salt, solvate or prodrug e.g. ester, of a compound of the invention, which upon administration to the recipient is capable of providing (directly or indirectly) a compound of the invention, or an active metabolite or residue thereof. Such derivatives are recognizable to those skilled in the art, without undue experimentation. Nevertheless, reference is made to the teaching of Burger's Medicinal Chemistry and Drug Discovery, 5^{th} Edition, Vol. 1: Principles and Practice, which is incorporated herein by reference to the extent of teaching such derivatives. In one embodiment of the invention the pharmaceutically acceptable derivatives are salts, solvates, esters, carbamates and phosphate esters. In another embodiment of the invention the pharmaceutically acceptable derivatives are salts, solvates and esters. In another embodiment of the invention the pharmaceutically acceptable derivatives are salts and esters, in particular salts.

The compounds of the present invention may be in the form of and/or may be administered as a pharmaceutically acceptable salt. For a review on suitable salts see Berge et al., J. Pharm. Sci., 1977, 66, 1-19.

Typically, a pharmaceutical acceptable salt may be readily prepared by using a desired acid or base as appropriate. The salt may precipitate from solution and be collected by filtration or may be recovered by evaporation of the solvent.

Salts of the compounds of the present invention may, for example, comprise acid addition salts resulting from reaction of an acid with a nitrogen atom present in a compound of formula (I). Salts encompassed within the term “pharmaceutically acceptable salts” refer to non-toxic salts of the compounds of this invention. Suitable addition salts are formed from acids which form non-toxic salts and examples are acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, ethanesulphonate, formate, fumarate, gluceptate, gluconate, glutamate, glycollylarsanilate, hexylresorcinate, hydrabamine, hydrobromide, hydrochloride, hydrogen phosphate, hydroiodide, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, oxaloacetate, pamoate (embonate), palmitate, pantothenate, phosphate/diphosphate, piruvate, polygalacturonate, saccharate, salicylate, stearate, subacetate, succinate, sulphate, tannate, tartrate, teoclate, tosylate, triethiodide, trifluoroacetate and valerate.

Pharmaceutically acceptable base salts include ammonium salts such as a trimethylammonium salt, alkali metal salts such as those of sodium and potassium, alkaline earth metal salts such as those of calcium and magnesium and salts with organic bases, including salts of primary, secondary and tertiary amines, such as isopropylamine, diethylamine, ethanolamine, trimethylamine, dicyclohexyl amine and N-methyl-D-glucamine.

Those skilled in the art of organic chemistry will appreciate that many organic compounds can form complexes with solvents in which they are reacted or from which they are precipitated or crystallized. These complexes are known as “solvates”. As used herein, the term “solvate” refers to a complex of variable stoichiometry formed by a solute (in this invention, a compound of formula (I) or a salt thereof) and a solvent. Such solvents for the purpose of the invention may not interfere with the biological activity of the solute. Examples of suitable solvents include water, methanol, ethanol and acetic acid. Preferably the solvent used is a pharmaceutically acceptable solvent. Examples of suitable pharmaceutically acceptable solvents include water, ethanol and acetic acid. Most preferably the solvent used is water. A complex with water is known as a “hydrate”. Solvates of the compound of the invention are within the scope of the invention.

As used herein, the term “prodrug” means a compound which is converted within the body, e.g. by hydrolysis in the blood, into its active form that has medical effects. Pharmaceutically acceptable prodrugs are described in T. Higuchi and V. Stella, Prodrugs as Novel Delivery Systems, Vol. 14 of the A.C.S. Symposium Series; Edward B. Roche, ed., Bioreversible Carriers in Drug Design, American Pharmaceutical Association and Pergamon Press, 1987; and in D. Fleisher, S. Ramon and H. Barbra “Improved oral drug delivery: solubility limitations overcome by the use of prodrugs”, Advanced Drug Delivery Reviews (1996) 19(2) 115-130, each of which are incorporated herein by reference.

Prodrugs are any covalently bonded carriers that release a compound of formula (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy or amine groups are bonded to any group that, when administered to a patient, cleaves to form the hydroxy or amine groups. Thus, representative examples of prodrugs include (but are not limited to) acetate, formate and benzoate derivatives of alcohol and amine functional groups of the compounds of formula (I). Further, in the case of a carboxylic acid (—COOH), esters may be employed, such as methyl esters, ethyl esters, and the like. Esters may be active in their own right and/or be hydrolysable under in vivo conditions in the human body. Suitable pharmaceutically acceptable in vivo hydrolysable ester groups include those which break down readily in the human body to leave the parent acid or its salt.

As used herein, the term “alkyl” refers to straight or branched hydrocarbon chains containing the specified number of carbon atoms. For example, C₁₋₆alkyl means a straight or branched alkyl containing at least 1, and at most 6, carbon atoms. Examples of “alkyl” as used herein include, but are not limited to, methyl, ethyl, n-propyl, n-butyl, n-pentyl, isobutyl, isopropyl, sec-butyl, t-butyl and hexyl and the like.

As used herein, the term “alkenyl” refers to straight or branched hydrocarbon chains containing the specified number of carbon atoms and containing at least one double bond. For example, C₂₋₆alkenyl means a straight or branched alkenyl containing at least 2, and at most 6, carbon atoms and containing at least one double bond. Examples of “alkenyl” as used herein include, but are not limited to ethenyl, 2-propenyl, 3-butenyl, 2-butenyl, 2-pentenyl, 3-pentenyl, 3-methyl-2-butenyl, 3-methylbut-2-enyl, 3-hexenyl, 1,1-dimethylbut-2-enyl and the like.

As used herein, the term “aryl” refers to phenyl and naphthyl.

As used herein, the term “alkoxy” refers to straight or branched chain alkoxy groups containing the specified number of carbon atoms. For example, C₁₋₆alkoxy means a straight or branched alkoxy containing at least 1, and at most 6, carbon atoms. Examples of “alkoxy” as used herein include, but are not limited to, methoxy, ethoxy, propoxy, prop-2-oxy, butoxy, but-2-oxy, 2-methylprop-1-oxy, 2-methylprop-2-oxy, pentoxy and hexyloxy.

As used herein, the term “cycloalkyl” refers to a non-aromatic hydrocarbon ring containing the specified number of carbon atoms. For example, C₃₋₇cycloalkyl means a non-aromatic ring containing at least three, and at most seven, ring carbon atoms. Representative examples of “cycloalkyl” as used herein include, but are not limited to, cyclopropyl, cyclobutyl, cyclopentyl, cyclohexyl and cycloheptyl.

As used herein, the terms “heteroaryl ring” and “heteroaryl” refer to a monocyclic five- to seven-membered unsaturated hydrocarbon ring containing at least one heteroatom selected from oxygen, nitrogen and sulfur. Examples of heteroaryl rings include, but are not limited to, furyl, thienyl, pyrrolyl, oxazolyl, thiazolyl, isoxazolyl, isothiazolyl, imidazolyl, pyrazolyl, oxadiazolyl, triazolyl, tetrazolyl, thiadiazolyl, pyridyl, pyridazinyl, pyrimidinyl, pyrazinyl and triazinyl. The terms “heteroaryl ring” and “heteroaryl” also refer to fused aromatic rings comprising at least one heteroatom selected from oxygen, nitrogen and sulfur. Preferably, the fused rings each have five or six ring atoms. Examples of fused aromatic rings include, but are not limited to, indolyl, isoindolyl, azaindolyl, benzoxazolyl, benzimidazolyl, benzothiazolyl, benzofuranyl, benzothiophenyl, quinolyl, isoquinolyl, quinazolinyl, cinnolinyl and phthalazinyl.

As used herein, the terms “heterocyclic rings” and “heterocyclyl” refer to a monocyclic three- to seven-membered saturated or non-aromatic, unsaturated hydrocarbon ring containing at least one heteroatom selected from oxygen, nitrogen and sulfur. Examples of heterocyclyl groups include, but are not limited to, aziridinyl, pyrrolinyl, pyrrolidinyl, imidazolinyl, imidazolidinyl, pyrazolinyl, pyrazolidinyl, piperidyl, piperazinyl, morpholino and thiomorpholino.

The term “aralkyl” or “heteroarylalkyl” or “heterocyclicalkyl” is used herein to mean a C₁₋₄ alkyl linkage, unless the carbon chain linkage is otherwise indicated which may be straight or branched, as defined above, and which carbon chain is attached to the aryl, cycloalkyl, heteroaryl or heterocyclic moiety.

As used herein, the terms “halogen” or “halo” refer to the elements fluorine, chlorine, bromine and iodine.

As used herein, the term “optionally” means that the subsequently described event(s) may or may not occur, and includes both event(s) which occur and events that do not occur.

As used herein, the term “substituted” refers to substitution with the named substituent or substituents, multiple degrees of substitution being allowed unless otherwise stated.

As used herein, “substituted” or “optionally substituted” unless specifically defined elsewhere shall mean such groups as halogen, such as fluorine, chlorine, bromine or iodine; hydroxy; hydroxy substituted C₁₋₆alkyl; C₁₋₆ alkoxy, such as methoxy or ethoxy; halosubstituted C₁₋₆ alkoxy; S(O)m′ alkyl, such as methyl thio, methylsulfinyl or methyl sulfonyl, wherein m′ is 0, 1 or 2; —C(O); NR₇R_{7′}, wherein R₇ and R_{7′} are each independently hydrogen or C₁₋₄ alkyl, such as amino or mono or -disubstituted C₁₋₄ alkyl or wherein the R₇R_{7′} can cyclize together with the nitrogen to which they are attached to form a 5 to 7 membered ring which optionally contains an additional heteroatom selected from O/N/S; C₁₋₆ alkyl, C₃₋₇cycloalkyl, or C₃₋₇cycloalkyl C₁₋₆ alkyl group, such as methyl, ethyl, propyl, isopropyl, t-butyl, etc. or cyclopropyl methyl; halosubstituted C₁₋₆ alkyl, such CF₂CF₂H, or CF₃; an optionally substituted aryl, such as phenyl, or an optionally substituted arylalkyl, such as benzyl or phenethyl, wherein these aryl containing moieties may also be substituted one to two times by halogen; hydroxy; hydroxy substituted alkyl; C₁₋₆ alkoxy; S(O)_{m′}alkyl; amino, mono & di-substituted C₁₋₄ alkyl amino; C₁₋₄ alkyl, or CF₃.

With regard to stereoisomers, the compounds of structure (I) may have one or more asymmetric carbon atom and may occur as racemates, racemic mixtures and as individual enantiomers or diastereomers. All such isomeric forms are included within the present invention, including mixtures thereof.

Cis (E) and trans (Z) isomerism may also occur. The present invention includes the individual stereoisomers of the compound of the invention and, where appropriate, the individual tautomeric forms thereof, together with mixtures thereof.

Separation of diastereoisomers or cis and trans isomers may be achieved by conventional techniques, e.g. by fractional crystallisation, chromatography or H.P.L.C. A stereoisomeric mixture of the agent may also be prepared from a corresponding optically pure intermediate or by resolution, such as H.P.L.C. of the corresponding racemate using a suitable chiral support or by fractional crystallisation of the diastereoisomeric salts formed by reaction of the corresponding racemate with a suitable optically active acid or base, as appropriate.

Furthermore, some of the crystalline forms of the compounds of structure (I) may exist as polymorphs, which are included in the present invention.

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the working Examples.

A compound of formula (I) may be prepared by reacting a compound of (II) 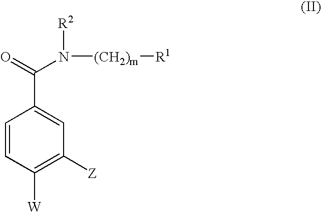
in which R¹, R², Z and m are as hereinbefore defined and W is halogen, in particular bromine or iodine for use in a Suzuki reaction, with a compound of formula (III) 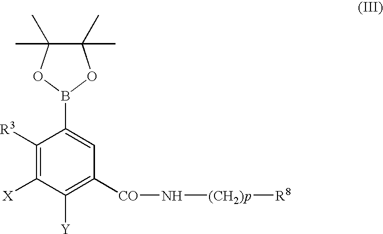
in which R³, p, X and Y are as hereinbefore defined, for formula (I), and wherein R⁸ is selected from hydrogen, C₁₋₆alkyl, C₃₋₇cycloalkyl optionally substituted by one or more C₁₋₆alkyl groups, CONHR⁹, phenyl optionally substituted by R¹³ and/or R¹⁴, and heteroaryl optionally substituted by R¹³ and/or R¹⁴, wherein R⁹, R¹³ and R¹⁴, are as defined in formula (I); in the presence of a catalyst, for example tetrakis(triphenylphosphine)palladium.

A compound of formula (II) may readily be prepared from a corresponding acid compound of formula (IV) 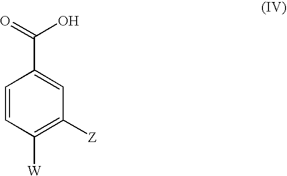
in which W and Z are hereinbefore defined,
by converting the acid to an activated form of the acid, for example the acid chloride, by treatment with, for example, thionyl chloride, and then reacting the activated acid thus formed with an amine compound of formula (V) 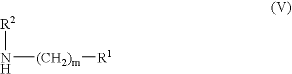
in which R¹, R² and m are as hereinbefore defined,
under amide forming conditions.

Suitable amide forming conditions are well known in the art and include treating a solution of the acid of formula (IV), or the activated form thereof, in for example acetone or dichloromethane, with an amine of formula (V) in the presence of sodium carbonate.

A compound of formula (III) may be prepared by reacting a compound of formula (VI) 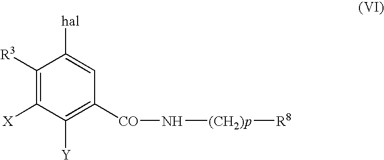
in which R³, R⁸, X and Y are as hereinbefore defined and hal is halogen, in particular bromine or iodine, with bis(pinnacolato)diboron, PdCl₂dppf and potassium acetate in a solvent such as DMF.

In another embodiment, a compound of formula (III) may be prepared by reacting an acid compound of formula (VII) 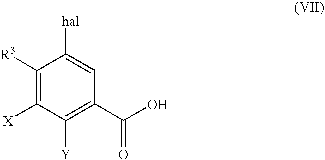
in which R³, hal, X and Y are as hereinbefore defined,
with bis(pinnacolato)diboron, PdCl₂dppf and potassium acetate in a solvent such as DMF, and then forming an amide by reaction with an amine compound of formula (V) as hereinbefore defined.

A compound of formula (I) may also be prepared by reacting a compound of formula (VII) 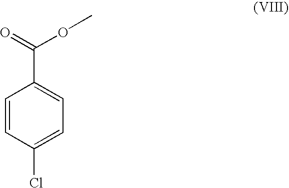
with a compound of formula (III) as hereinbefore defined and then reacting the acid thus formed with an amine of formula (V) as hereinbefore defined, under amide forming conditions.

Additionally, a compound of formula (I) may be prepared by reacting a compound of (II) as hereinbefore defined with a compound of formula (IX) 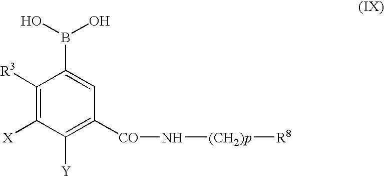
in which R³, X and Y are as hereinbefore defined,
in the presence of a catalyst, for example tetrakis(triphenylphosphine)palladium.

Additionally, a compound of formula (I) may be prepared by reacting a compound of formula (X) 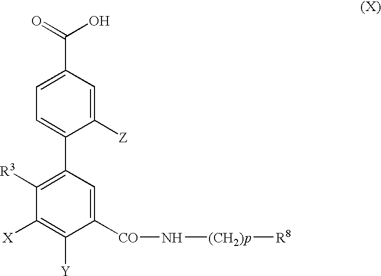
in which R³, X, Y, Z and n are as hereinbefore defined,
with an amine compound of formula (V) as hereinbefore defined,
under amide forming conditions.

A compound of formula (X) may be prepared by reacting a compound of formula (XI) 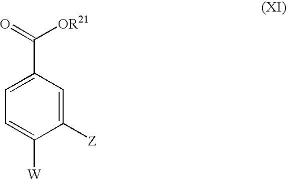
in which W, Z and n are as hereinbefore defined and R₂₁ is C₁₋₆alkyl, in particular methyl or ethyl, with a compound of formula (III) or a compound of formula (IX) as hereinbefore defined, in the presence of a catalyst, for example tetrakis(triphenylphosphine)palladium, and removing the R₂₁ group, if necessary, by treatment with a base such as sodium hydroxide in a solvent such as methanol.

A further general method comprises final stage modification of one compound of formula (I) into another compound of formula (I). Suitable functional group transformations for converting one compound of formula (I) into another compound of formula (I) are well known in the art and are described in, for instance, Comprehensive Heterocyclic Chemistry II, eds. A. R. Katritzky, C. W. Rees and E. F. V. Scriven (Pergamon Press, 1996), Comprehensive Organic Functional Group Transformations, eds. A. R. Katritzky, O. Meth-Cohn and C. W. Rees (Elsevier Science Ltd., Oxford, 1995), Comprehensive Organic Chemistry, eds. D. Barton and W. D. Ollis (Pergamon Press, Oxford, 1979), and Comprehensive Organic Transformations, R. C. Larock (VCH Publishers Inc., New York, 1989).

Alternatively, a compound of formula (I) may be prepared from a compound of formula (XII) 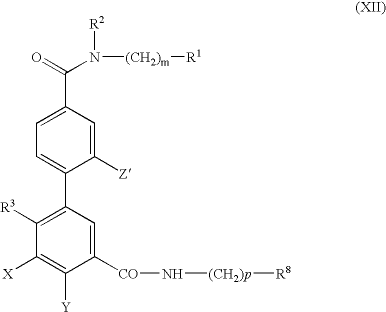
in which Z′ is a group convertible to Z as defined for formula (I). Conversion of a Z′ group may arise if, for example, an alternative group such as a halogen group or a protecting group is present during the reactions described above. A comprehensive discussion of protecting groups and methods for cleaving protected derivatives is given in for example T. W. Greene and P. G. M Wuts in Protective Groups in Organic Synthesis 2^{nd} ed., John Wiley & Son, Inc 1991.

For example, another general method for preparing the compounds of formula (I) comprises the reactions set out in Scheme 1 below.

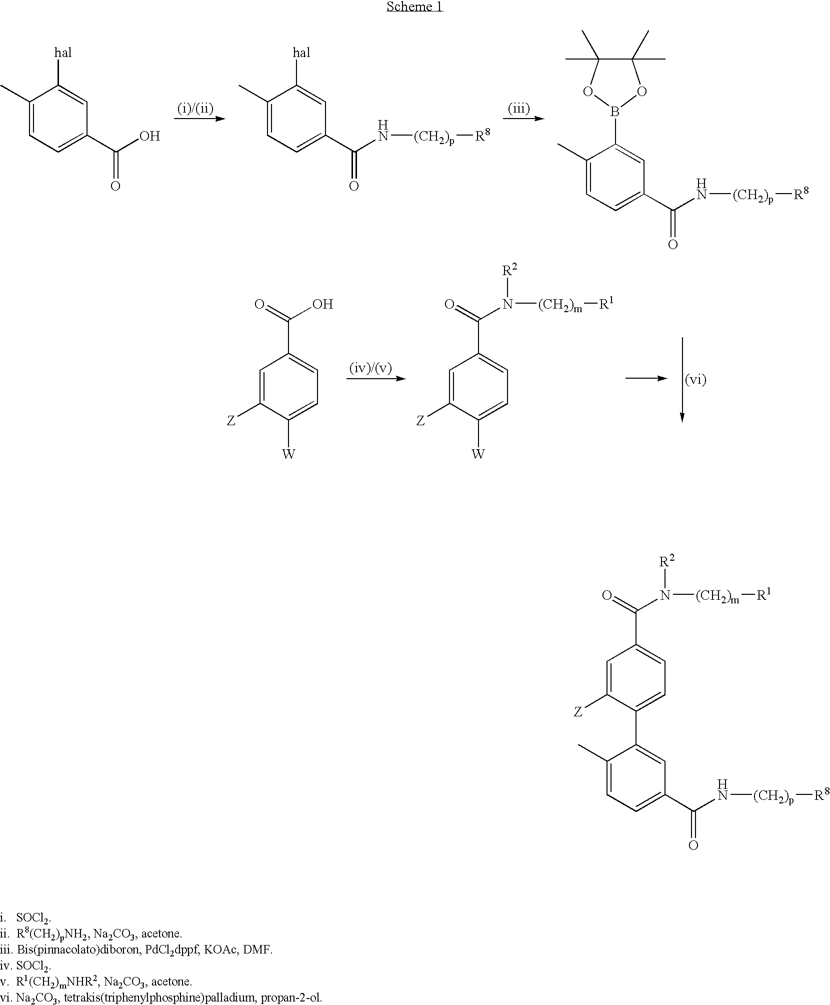

For example, another general method for preparing the compounds of formula (I) comprises the reactions set out in Scheme 2 below.

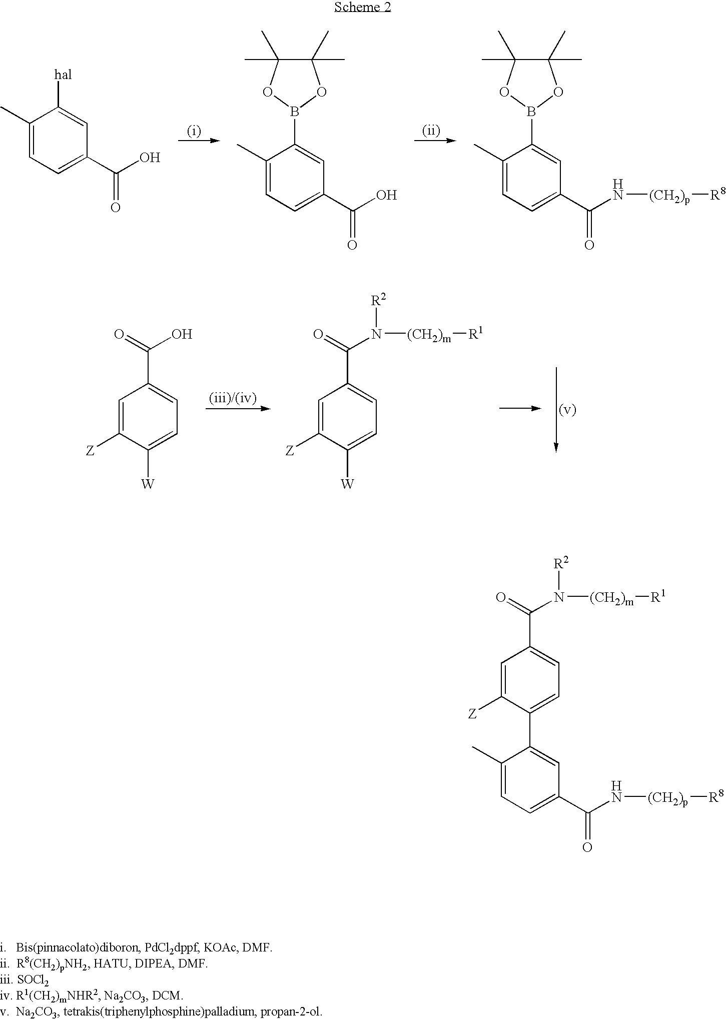

For example, another general method for preparing the compounds of formula (I) comprises the reactions set out in Scheme 3 below.

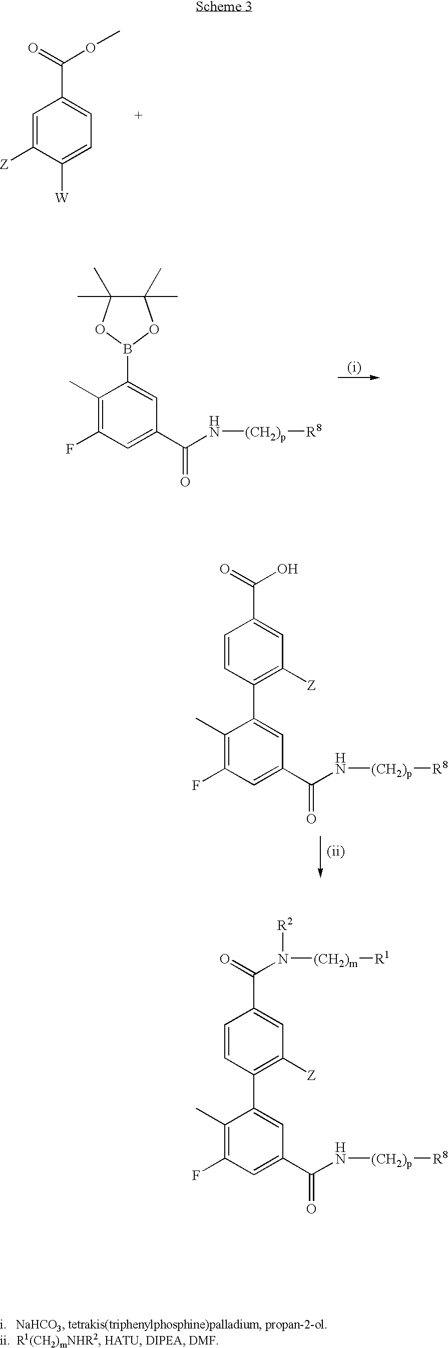

For example, another general method for preparing the compounds of formula (I) comprises the reactions set out in Scheme 4 below.

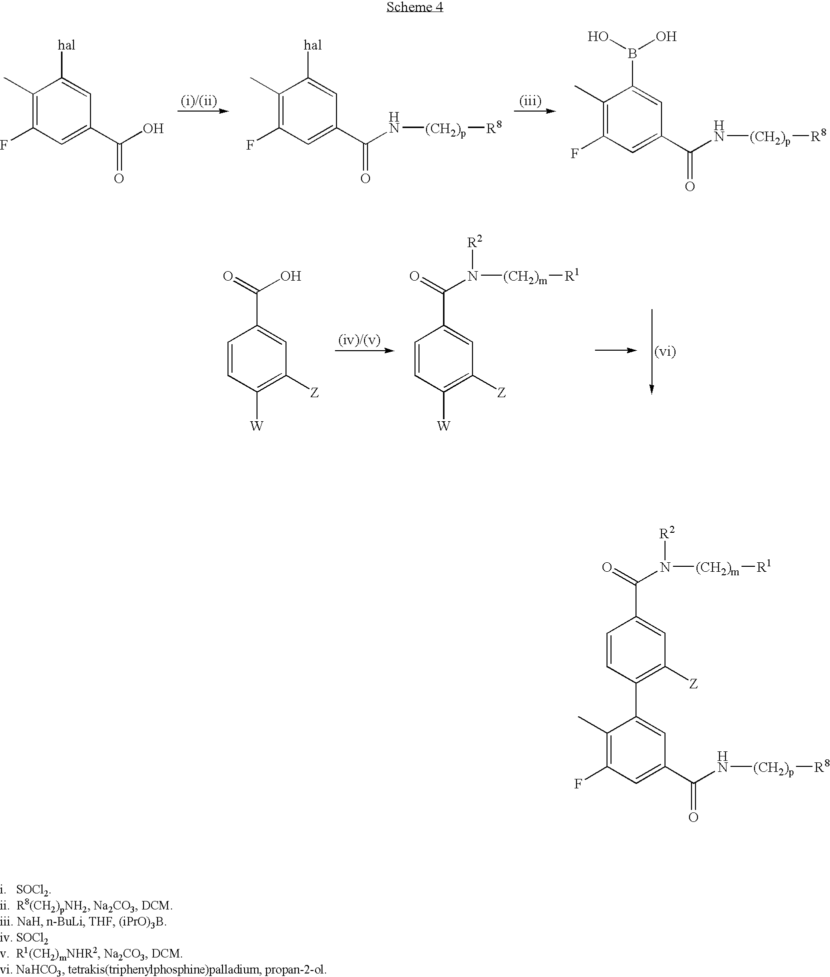

For example, another method for preparing the compounds of formula (I) comprises the reactions set out in Scheme 5 below.

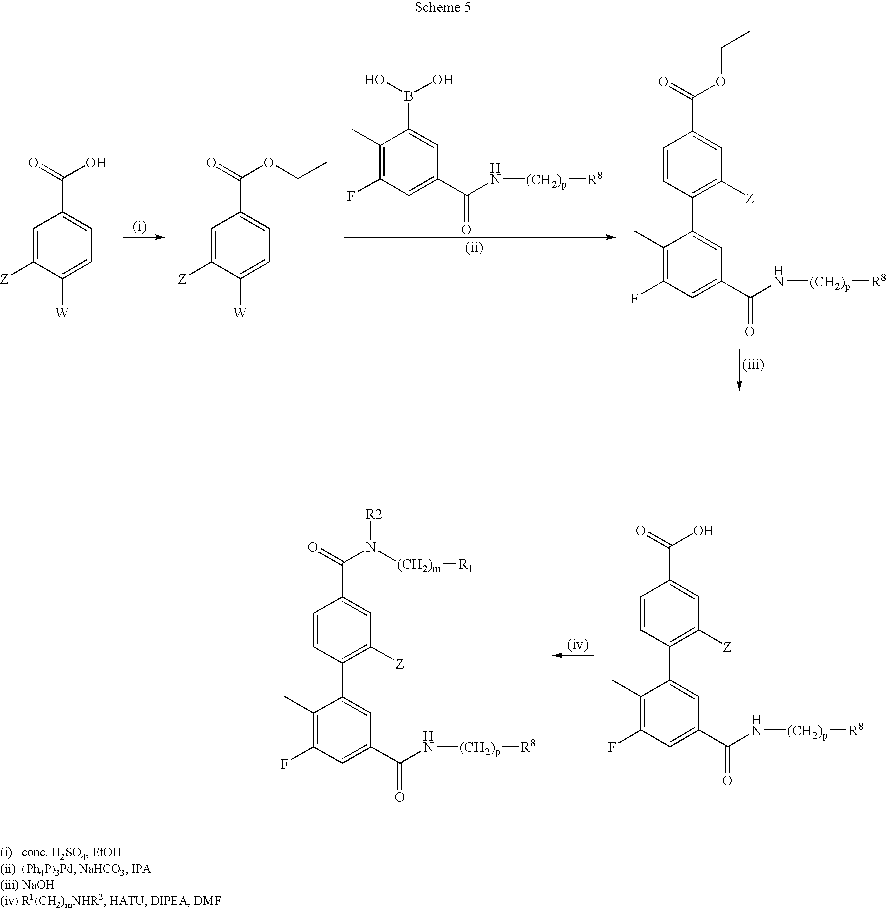

For example, another method for preparing the compounds of formula (I) comprises the reactions set out in Scheme 6 below.

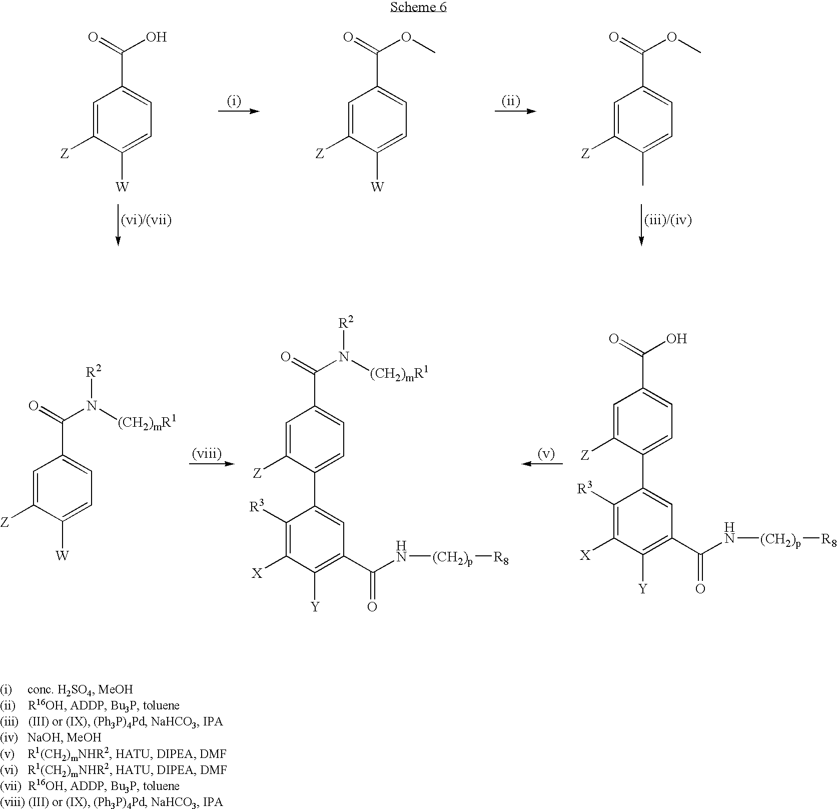

For example, another method for preparing the compounds of formula (I) comprises the reactions set out in Scheme 7 below. It includes the functional group transformations of Z′ to Z in step (ix) and these transformations may include more than one step in the synthesis. Examples of these transformations include, but not limited to: reaction of amine (e.g., Z′=NH₂) with acid (or acid chloride) to form amide, reaction of amine (e.g., Z′=NH₂) with isocyanate to form urea, reaction of acid (e.g., Z′=—COOH) with amine to form amide.

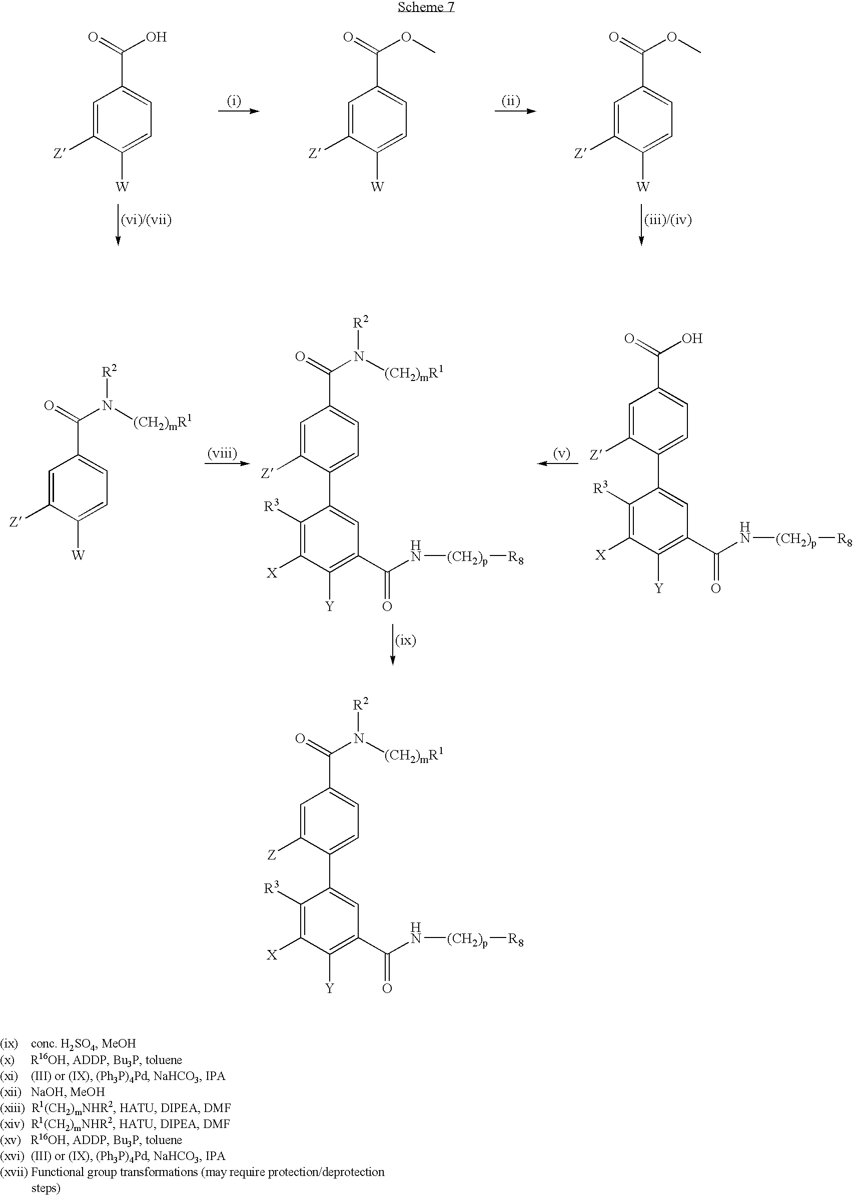

Those skilled in the art will appreciate that in the preparation of the compound of the invention or a solvate thereof it may be necessary and/or desirable to protect one or more sensitive groups in the molecule to prevent undesirable side reactions. Suitable protecting groups for use according to the present invention are well known to those skilled in the art and may be used in a conventional manner. See, for example, “Protective groups in organic synthesis” by T. W. Greene and P. G. M. Wuts (John Wiley & sons 1991) or “Protecting Groups” by P. J. Kocienski (Georg Thieme Verlag 1994). Examples of suitable amino protecting groups include acyl type protecting groups (e.g. formyl, trifluoroacetyl, acetyl), aromatic urethane type protecting groups (e.g. benzyloxycarbonyl (Cbz) and substituted Cbz), aliphatic urethane protecting groups (e.g. 9-fluorenylmethoxycarbonyl (Fmoc), t-butyloxycarbonyl (Boc), isopropyloxycarbonyl, cyclohexyloxycarbonyl) and alkyl type protecting groups (e.g. benzyl, trityl, chlorotrityl). Examples of suitable oxygen protecting groups may include for example alky silyl groups, such as trimethylsilyl or tert-butyldimethylsilyl; alkyl ethers such as tetrahydropyranyl or tert-butyl; or esters such as acetate.

Methods of Treatment

The compounds of Formula (I), or a pharmaceutically acceptable salt or derivative thereof, can be used in the manufacture of a medicament for the prophylactic or therapeutic treatment of any disease state in a human, or other mammal, in which and underlying inflammatory condition is demonstrated by, exacerbated by, and amplified by excessive cytokine, chemokine and adhesion molecule production by such mammal's cell, such as, but not limited to, monocytes, macrophages, neutrophils, endothelial cells or smooth muscle cells.

The compounds of the present invention are generally inhibitors of the serine/threonine kinase p38 and are therefore also inhibitors of cytokine production which is mediated by p38 kinase. Within the meaning of the term “inhibitors of the serine/threonine kinase p38” are included those compounds that interfere with the ability of p38 to transfer a phosphate group from ATP to a protein substrate according to the assay described below.

It will be appreciated that the compounds of the invention may be selective for one or more of the isoforms of p38, for example p38α, p38β, p38γ and/or p38δ. In one embodiment, the compounds of the invention selectively inhibit the p38α isoform. In another embodiment, the compounds of the invention selectively inhibit the p38β isoform. In a further embodiment, the compounds of the invention selectively inhibit the p38α and p38β isoforms. Assays for determining the selectivity of compounds for the p38 isoforms are described in, for example, WO 99/61426, WO 00/71535 and WO 02/46158. It is known that p38 kinase activity can be elevated (locally or throughout the body), p38 kinase can be incorrectly temporally active or expressed, p38 kinase can be expressed or active in an inappropriate location, p38 kinase can be constitutively expressed, or p38 kinase expression can be erratic; similarly, cytokine production mediated by p38 kinase activity can be occurring at inappropriate times, inappropriate locations, or it can occur at detrimentally high levels.

Accordingly, the present invention provides a method for the treatment of a condition or disease state mediated by p38 kinase activity, or mediated by cytokines produced by the activity of p38 kinase, in a subject which comprises administering to said subject a therapeutically effective amount of a compound of formula (I) or a pharmaceutically acceptable derivative thereof. The compound may be administered as a single or polymorphic crystalline form or forms, an amorphous form, a single enantiomer, a racemic mixture, a single stereoisomer, a mixture of stereoisomerism, a single diastereoisomer or a mixture of diastereoisomers.

The present invention treats these conditions by providing a therapeutically effective amount of a compound of this invention. By “therapeutically effective amount” is meant a symptom-alleviating or symptom-reducing amount, a cytokine-reducing amount, a cytokine-inhibiting amount, a kinase-regulating amount and/or a kinase-inhibiting amount of a compound. Such amounts can be readily determined by standard methods, such as by measuring cytokine levels or observing alleviation of clinical symptoms. For example, the clinician can monitor accepted measurement scores for anti-inflammatory treatments. It will be appreciated that reference to treatment includes acute treatment or prophylaxis as well as the alleviation of established symptoms.

Compounds of Formula (I) are capable of inhibiting proinflammatory mediators in the form of cytokines or chemokines, such as IL-1, IL-6, IL-8, TNF and RANTES and are therefore of use in therapy for diseases with an inflammatory component. IL-1, IL-6, IL-8, RANTES and TNF as well as other cytokines and chemokines affect a wide variety of cells and tissues, are important and critical inflammatory mediators of a wide variety of disease states and conditions. These pro-inflammatory mediators are produced by leukocytes as well as epithelial cells, endothelial cells, smooth muscle cells and other resident cells. The inhibition of these pro-inflammatory cytokines and chemokines is of benefit in controlling, reducing and alleviating many disease states which are marked by the excess production of these mediators.

Accordingly, the present invention provides a method of treating an inflammatory disease, which comprises administering an effective cytokine-interfering amount of a compound of Formula (I) or a pharmaceutically acceptable salt thereof. In particular, compounds of Formula (I) or a pharmaceutically acceptable salt thereof are of use in the prophylaxis or therapy of any disease state in a human, or other mammal, which is exacerbated by or caused by excessive inflammatory mediator production, such as IL-1, IL-6, IL-8 or TNF, by such mammal's cells, such as, but not limited to, monocytes, macrophages, neutrophils and endothelial cells. Excess cytokine and chemokine production are the biomarkers of inflammatory diseases, and can be measured in serum or tissue samples from patients affected with these diseases.

Excessive cytokine production is implicated in exacerbating and amplifying or perpetuating inflammatory diseases which include rheumatoid arthritis, osteoarthritis, meningitis, ischemic and hemorrhagic stroke, neurotrauma/closed head injury, stroke, endotoxemia and/or toxic shock syndrome, other acute or chronic inflammatory disease states such as the inflammatory reaction induced by endotoxin or inflammatory bowel disease, tuberculosis, atherosclerosis and associated disorders (mycardial infarction and stroke), muscle degeneration, pulmonary inflammation in asthma or chronic obstructive pulmonary disease (COPD), multiple sclerosis, cachexia, bone resorption, psoriatic arthritis, Reiter's syndrome, gout, traumatic arthritis, rubella arthritis and acute synovitis, progressive renal disease (diabetic and non-diabetic), hypertension and salt-sensitive hypertension. Recent evidence also links inflammation as measured by excess IL-1 levels to diabetes, pancreatic β cell diseases and Alzheimer's disease.

Use of a p38 inhibitor compound for the treatment of p38 mediated disease states, can include, but not be limited to neurodegenerative diseases, such as Alzheimer's disease (as noted above), Parkinson's disease and multiple sclerosis, etc.

In a further aspect, this invention relates to a method of inhibiting the production of TNF in a mammal in need thereof which comprises administering to said mammal an effective amount of a compound of Formula (I) or a pharmaceutically acceptable salt thereof.

Excessive or unregulated TNF production has been implicated in mediating or exacerbating a number of inflammatory diseases including rheumatoid arthritis, rheumatoid spondylitis, osteoarthritis, gouty arthritis and other arthritic conditions, sepsis, septic shock, endotoxic shock, gram negative sepsis, toxic shock syndrome, adult respiratory distress syndrome, chronic obstructive pulmonary disease, silicosis, pulmonary sarcoisosis, bone resorption diseases, such as osteoporosis, cardiac, brain and renal reperfusion injury, graft vs. host reaction, allograft rejections, fever and myalgias due to infection, such as influenza, brain infections including encephalitis (including HIV-induced forms), cerebral malaria, meningitis, ischemic and hemorrhagic stroke, cachexia secondary to infection or malignancy, cachexia secondary to acquired immune deficiency syndrome (AIDS), AIDS, ARC (AIDS related complex), keloid formation, scar tissue formation, inflammatory bowel disease, Crohn's disease, ulcerative colitis and pyresis.

Compounds of Formula (I) are capable of inhibiting inducible proinflammatory proteins, such as COX-2, also referred to by many other names such as prostaglandin endoperoxide synthase-2 (PGHS-2) and iNOS and are therefore of use in therapy. Proinflammatory lipid mediators of the cyclooxygenase (CO) pathway are produced by the inducible COX-2 enzyme. Regulation, therefore of COX-2 which is responsible for the these products derived from arachidonic acid, such as prostaglandins affect a wide variety of cells and tissues are important and critical inflammatory mediators of a wide variety of disease states and conditions. Expression of COX-1 is not effected by compounds of Formula (I). This selective inhibition of COX-2 may alleviate or spare ulcerogenic liability associated with inhibition of COX-1 thereby inhibiting prostaglandins essential for cytoprotective effects. Thus inhibition of these pro-inflammatory mediators is of benefit in controlling, reducing and alleviating many of these disease states. Most notably these inflammatory mediators, in particular prostaglandins, have been implicated in pain, such as in the sensitization of pain receptors, or edema. This aspect of pain management therefore includes treatment of neuromuscular pain, headache, cancer pain, arthritis pain and dental pain. Compounds of Formula (I) or a pharmaceutically acceptable salt thereof, are of use in the prophylaxis or therapy in a human, or other mammal, by inhibition of the synthesis of the COX-2 enzyme.

Accordingly, the present invention provides a method of inhibiting the synthesis of COX-2 which comprises administering an effective amount of a compound of Formula (I) or a pharmaceutically acceptable salt thereof. The present invention also provides for a method of prophylaxis treatment in a human, or other mammal, by inhibition of the synthesis of the COX-2 enzyme.

Compounds of Formula (I) are also useful in the treatment of viral infections, where such viruses are sensitive to upregulation by TNF or will elicit TNF production in vivo. The viruses contemplated for treatment herein are those that produce TNF as a result of infection, or those which are sensitive to inhibition, such as by decreased replication, directly or indirectly, by the TNF inhibiting-compounds of Formula (I). Such viruses include, but are not limited to HIV-1, HIV-2 and HIV-3, Cytomegalovirus (CMV), Influenza, adenovirus and the Herpes group of viruses, such as but not limited to, Herpes Zoster and Herpes Simplex. Accordingly, in a further aspect, this invention relates to a method of treating a mammal afflicted with a human immunodeficiency virus (HIV) which comprises administering to such mammal an effective TNF inhibiting amount of a compound of Formula (I) or a pharmaceutically acceptable salt thereof.

Compounds of Formula (I) are also useful in treatment of the host response to additional viral infections. This additional aspect of the present invention is a method of treating the common cold or respiratory viral infection caused by human rhinovirus (HRV), other enteroviruses, coronavirus, influenza virus, parainfluenza virus, respiratory syncytial virus, or adenovirus in a human in need thereof which method comprises administering to said human an effective amount of a CBSP/p38 inhibitor. It should be noted that the treatment herein is not directed to the elimination or treatment of the viral organism itself but is directed to treatment of the respiratory viral infection that exacerbates other diseases or symptoms of disease, such as asthma (exacerbated by such infections), chronic bronchitis, chronic obstructive pulmonary disease, otitis media, and sinusitis.

Another aspect of the present invention is a method of treating, including prophylaxis of influenza induced pneumonia in a human in need thereof which method comprises administering to said human an effective amount of a CBSP/p38 inhibitor.

The present invention also relates to the use of the CSBP/p38 kinase inhibitor for the treatment, including prophylaxis, of inflammation associated with a viral infection of a human rhinovirus (HRV), other enteroviruses, coronavirus, influenza virus, parainfluenza virus, respiratory syncytial virus, or adenovirus.

In particular, the present invention is directed to the treatment of a viral infection in a human, which is caused by the human rhinovirus (HRV), other enterovirus, coronavirus, influenza virus, parainfluenza virus, respiratory syncytial virus, or an adenovirus. In particular the invention is directed to respiratory viral infections that exacerbate asthma (induced by such infections), chronic bronchitis, chronic obstructive pulmonary disease, otitis media, and sinusitis. While inhibiting IL-8 or other cytokines may be beneficial in treating a rhinovirus may be known, the use of an inhibitor of the p38 kinase for treating HRV or other respiratory viral infections causing the common cold is believed novel. It should be noted that the respiratory viral infection treated herein may also be associated with a secondary bacterial infection, such as otitis media, sinusitis, or pneumonia.

For use herein treatment may include prophylaxis for use in a treatment group susceptible to such infections. It may also include reducing the symptoms of, ameliorating the symptoms of, reducing the severity of, reducing the incidence of, or any other change in the condition of the patient, which improves the therapeutic outcome. A preferred viral infection for treatment herein is the human rhinovirus (HRV) or respiratory syncytial virus (RSV).

It is also recognized that both IL-6 and IL-8 and other chemokines such as RANTES are produced during rhinovirus (HRV) infections and contribute to the pathogenesis of common cold and exacerbation of asthma associated with HRV infection (Turner et al. (1998), Clin. Infec. Dis., Vol. 26, p 840; Teren et al. (1997), Am J Respir Crit Care Med, Vol. 155, p1362; Grunberg et al. (1997), Am J Respir Crit Care Med 156:609 and Zhu et al, J Clin Invest (1996), 97:421). It has also been demonstrated in vitro that infection of pulmonary epithelial cells with HRV results in production of IL-6 and IL-8 (Subauste et al., J. Clin. Invest. 1995, 96:549.) Epithelial cells represent the primary site of infection of HRV. Therefore another aspect of the present invention is a method of treatment to reduce inflammatory response associated with a rhinovirus infection, not necessarily a direct effect on virus itself.

Compounds of Formula (I) may also be used in association with the veterinary treatment of mammals, other than in humans, in need of inhibition of TNF production. TNF mediated diseases for treatment, therapeutically or prophylactically, in animals include disease states such as those noted above, but in particular viral infections. Examples of such viruses include, but are not limited to, lentivirus infections such as, equine infectious anaemia virus, caprine arthritis virus, visna virus, or maedi virus or retrovirus infections, such as but not limited to feline immunodeficiency virus (FIV), bovine immunodeficiency virus, or canine immunodeficiency virus or other retroviral infections.

Compounds of Formula (I) have also been shown to inhibit the production of IL-8 (Interleukin-8, NAP) and other chemokines. Accordingly, in a further aspect, this invention relates to a method of inhibiting the production of IL-8 in a mammal in need thereof which comprises administering to said mammal an effective amount of a compound of Formula (I) or a pharmaceutically acceptable salt thereof.

There are many disease states in which excessive or unregulated IL-8 production is implicated in exacerbating and/or causing the disease. These diseases are characterized by massive lymphocyte infiltration, especially of neutrophils, such as, psoriasis, inflammatory bowel disease, asthma, cardiac, brain and renal reperfusion injury, adult respiratory distress syndrome, thrombosis and glomerulonephritis. All of these diseases are associated with increased IL-8 production which is responsible for the chemotaxis of neutrophils into the inflammatory site. In contrast to other inflammatory cytokines (IL-1, TNF, and IL-6), IL-8 has the unique property of promoting neutrophil chemotaxis and activation. Therefore, the inhibition of IL-8 production would lead to a direct reduction in the neutrophil infiltration.

In addition to inflammation marked by increases in cytokine and chemokine production, chronic stresses such as those which activate p38 enzyme also lead to inappropriate cell growth and tissue repair. An additional aspect of the invention is treatment of chronic diseases which have an inappropriate angiogenic component including various ocular neovascularizations, such as diabetic retinopathy and macular degeneration. Other chronic diseases which have an excessive or increased proliferation of vasculature are tumor growth and metastasis, atherosclerosis, and certain arthritic conditions. Therefore p38 kinase inhibitors will be of utility in the blocking of the angiogenic component of these disease states.

The term “excessive or increased proliferation of vasculature inappropriate angiogenesis” as used herein includes, but is not limited to, diseases which are characterized by hemangiomas and ocular diseases.

The term “inappropriate angiogenesis” as used herein includes, but is not limited to, diseases which are characterized by vesicle proliferation with accompanying tissue proliferation, such as occurs in cancer, metastasis, arthritis and atherosclerosis.

Diseases of inappropriate repair include chronic diseases which have a remodeling or fibrotic component, such as renal fibrosis, chronic obstructive pulmonary disease, or idiopathic pulmonary fibrosis as a result of TGF-beta stimulation or other stimulation mediated by p38. Inappropriate remodeling refers to excess deposition of collagen or other matrix components leading to loss of elasticity, tissue damage and scarring, and consequent impairment of tissue function.

In addition to those diseases already noted, treatment of stroke, neurotrauma, cardiac and renal reperfusion injury, congestive heart failure, coronary arterial bypass grafting (CABG) surgery, chronic renal failure, angiogenesis & related processes, such as cancer, thrombosis, glomerulonephritis, diabetes and pancreatic cells, multiple sclerosis, muscle degeneration, eczema, psoriasis, sunburn, and conjunctivitis are also included as treatable by inhibition of p38 MAP kinase by anti-inflammatory mechanisms.

P38 inhibitors are effective in many in vivo animal models of the complex diseases identified with an inflammatory component. P38 inhibitors are effective in models of arthritis such as the collagen-induced arthritis model, adjuvant arthritis model and PGPS model. Inhibition of TNF production in the endotoxic shock model. Where the reduction in plasma level of TNF correlated with survival and protection from endotoxic shock related mortality. Inhibitors of p38 kinase activity are effective in inhibiting bone resorption in a rat fetal long bone organ culture system, in inhibition of indices of pain in models of neurogenic pain, in inhibition of pulmonary inflammation in models such as ozone-induced mucin production, LPS induced neutrophilia; in models of fibrosis such as bleomycin induced lung fibrosis or TGFbeta induced renal fibrosis. These models represent specific aspects of the diseases identified in the preceding sections. Efficacy in these models are indicative of expected therapeutic efficacy on markers and disease states caused by stimuli that cause pathological activation of the p38 enzyme. [Griswold et al., (1988) Arthritis Rheum. 31:1406-1412; Badger, et al., (1989) Circ. Shock 27, 51-61; Votta et al., (1994) in vitro. Bone 15, 533-538; Lee et al., (1993). B Ann. N.Y. Acad. Sci. 696, 149-170.; Ravingerova, et al. (2003) Mol. Cell. Biochem. 247:127-138; Valen (2003) Annals Med 35:300-3-7; Donnelly & Rogers (2003) Drugs 63:1973-1998; Newton & Holden (2003) Biodrugs 17:113-19; Barone & Parsons (2000) Exp. Opin. Invest. Drugs 9:2281-2306; also refs within Background section.]

The compounds of Formula (I) may also be used topically in the treatment or prophylaxis of topical disease states mediated by or exacerbated by excessive cytokine production, such as by IL-1 or TNF respectively, such as inflamed joints, eczema, psoriasis and other inflammatory skin conditions such as sunburn; inflammatory eye conditions including conjunctivitis; pyresis, pain and other conditions associated with inflammation. Periodontal disease has also been implicated in cytokine production, both topically and systemically. Hence use of compounds of Formula (I) to control the inflammation associated with cytokine production in such peroral diseases such as gingivitis and periodontitis is another aspect of the present invention. In addition, topical treatment could include delivery to the lung of a compound of Formula (I) for treatment of pulmonary diseases such as COPD, chronic bronchitis, emphysema, idiopathic pulmonary fibrosis, or asthma.

The compounds of Formula (I) are administered in an amount sufficient to inhibit markers of inflammation such as cytokine or chemokine, in particular IL-1, IL-6, IL-8 or TNF, production such that it is regulated down to therapeutically effective levels, which could be lower than the elevated disease levels, or as low as normal clinical levels, or in some cases to subnormal levels, so as to ameliorate or prevent the disease state. Abnormal or elevated levels of IL-1, IL-6, IL-8 or TNF, for instance in the context of the present invention, constitute: (i) levels of free (not cell bound) IL-1, IL-6, IL-8 or TNF greater than or equal to 1 picogram per ml; (ii) any cell associated IL-1, IL-6, IL-8 or TNF; or (iii) the presence of IL-1, IL-6, IL-8 or TNF mRNA above basal levels in cells or tissues in which IL-1, IL-6, IL-8 or TNF, respectively, is produced.

The discovery that the compounds of Formula (I) are inhibitors of cytokines, specifically IL-1, IL-6, IL-8 and TNF is based upon the effects of the compounds of Formulas (I) on the production of the IL-1, IL-8 and TNF in in vitro assays which are described herein.

As used herein, the term “inhibiting the production of IL-1 (IL-6, IL-8 or TNF)” refers to:

a) a decrease of excessive in vivo levels of the cytokine (IL-1, IL-6, IL-8 or TNF) in a human to normal or sub-normal levels by inhibition of the in vivo release of the cytokine by cells, including but not limited to monocytes, macrophages, endothelial cells, or neutrophils;

b) a down regulation, at the genomic level, of excessive in vivo levels of the cytokine (IL-1, IL-6, IL-8 or TNF) in a human to normal or sub-normal levels;

c) a down regulation, by inhibition of the direct synthesis of the cytokine (IL-1, IL-6, IL-8 or TNF) as a posttranslational event; or

d) a down regulation, at the translational level, of excessive in vivo levels of the cytokine (IL-1, IL-6, IL-8 or TNF) in a human to normal or sub-normal levels.

As used herein, the term “TNF mediated disease or disease state” refers to any and all disease states in which TNF plays a role, either by production of TNF itself, or by TNF causing another monokine to be released, such as but not limited to IL-1, IL-6 or IL-8. A disease state in which, for instance, IL-1 is a major component, and whose production or action, is exacerbated or secreted in response to TNF, would therefore be considered a disease state mediated by TNF. In addition, TNF plays a role in disease such as stroke, for instance, through initiation of apoptosis of target cells, causing tissue damage. Reduction of TNF levels will also ameliorate the tissue damage.

As used herein, the term “cytokine” refers to any secreted polypeptide that affects the functions of cells and is a molecule, which modulates interactions between cells in the immune, inflammatory or hematopoietic response. A cytokine includes, but is not limited to, monokines and lymphokines, regardless of which cells produce them. For instance, a monokine is generally referred to as being produced and secreted by a mononuclear cell, such as a macrophage and/or monocyte. Many other cells however also produce monokines, such as natural killer cells, fibroblasts, basophils, neutrophils, endothelial cells, brain astrocytes, bone marrow stromal cells, epidermal keratinocytes and B-lymphocytes. Lymphokines are generally referred to as being produced by lymphocyte cells. Examples of cytokines include, but are not limited to, Interleukin-1 (IL-1), Interleukin-6 (IL-6), Interleukin-8 (IL-8), Tumor Necrosis Factor-alpha (TNF-α) and Tumor Necrosis Factor beta (TNF-β).

As used herein, the term “cytokine interfering” or “cytokine suppressive amount” refers to an effective amount of a compound of Formula (I) which will cause a decrease in the in vivo levels of the cytokine to normal or sub-normal levels, when given to a patient for the prophylaxis or treatment of a disease state which is exacerbated by, or caused by, excessive or unregulated cytokine production.

As used herein, the cytokine referred to in the phrase “inhibition of a cytokine, for use in the treatment of a HIV-infected human” is a cytokine which is implicated in (a) the initiation and/or maintenance of T cell activation and/or activated T cell-mediated HIV gene expression and/or replication and/or (b) any cytokine-mediated disease associated problem such as cachexia or muscle degeneration.

As TNF-β (also known as lymphotoxin) has close structural homology with TNF-α (also known as cachectin) and since each induces similar biologic responses and binds to the same cellular receptor, both TNF-α and TNF-β are inhibited by the compounds of the present invention and thus are herein referred to collectively as “TNF” unless specifically delineated otherwise.

Accordingly, the present invention provides a method of treating a p38 kinase mediated disease in a mammal in need thereof, preferably a human, which comprises administering to said mammal, an effective amount of a compound of Formula (I) or a pharmaceutically acceptable salt thereof.

As noted, the compounds of Formula (I) may be used in combination with other therapeutically active ingredients. These combinations may conveniently be presented for use in the form of a pharmaceutical formulation and thus pharmaceutical formulations comprising a combination as defined above together with a pharmaceutically acceptable carrier or excipient comprise a further aspect of the invention.

The individual components of such combinations may be administered either sequentially or simultaneously in separate or combined pharmaceutical formulations by any convenient route.

When administration is sequential, either the compound of the invention or the second therapeutic agent may be administered first. When administration is simultaneous, the combination may be administered either in the same or different pharmaceutical composition.

When combined in the same formulation it will be appreciated that the two compounds must be stable and compatible with each other and the other components of the formulation. When formulated separately they may be provided in any convenient formulation, conveniently in such manner as are known for such compounds in the art.

In order to use a compound of Formula (I) or a pharmaceutically acceptable salt thereof in therapy, it will normally be formulated into a pharmaceutical composition in accordance with standard pharmaceutical practice with regard to the intended route of administration and standard pharmaceutical practice. This invention, therefore, also relates to a pharmaceutical composition comprising an effective, non-toxic amount of a compound of Formula (I) and a pharmaceutically acceptable excipients, carrier or diluent.

Compounds of Formula (I), pharmaceutically acceptable salts thereof and pharmaceutical compositions incorporating such may conveniently be administered by any of the routes conventionally used for drug administration, for instance, orally, topically, parentally or by inhalation.

The compounds of Formula (I) may be administered in conventional dosage forms prepared by combining a compound of Formula (I) with standard pharmaceutical carriers according to conventional procedures. The compounds of Formula (I) may also be administered in conventional dosages in combination with a known, second therapeutically active compound. These procedures may involve mixing, granulating and compressing or dissolving the ingredients as appropriate to the desired preparation. It will be appreciated that the form and character of the pharmaceutically acceptable character or diluent is dictated by the amount of active ingredient with which it is to be combined, the route of administration and other well-known variables. The carrier(s) must be “acceptable” in the sense of being compatible with the other ingredients of the formulation and not deleterious to the recipient thereof.

The pharmaceutical compositions may be for human or animal usage in human and veterinary medicine and will typically comprise any one or more of a pharmaceutically acceptable excipient, diluent or carrier. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical excipient, diluent or carrier can be selected with regard to the intended route of administration and standard pharmaceutical practice. The pharmaceutical compositions may comprise, or in addition to, the excipient, diluent or carrier any suitable binder(s), lubricant(s), suspending agent(s), coating agent(s) and solubilizing agent(s).

The pharmaceutical carrier employed may be, for example, either a solid or liquid. Exemplary of solid carriers are lactose, terra alba, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time delay material well known to the art, such as glyceryl mono-stearate or glyceryl distearate alone or with a wax.

A wide variety of pharmaceutical forms can be employed. Thus, if a solid carrier is used, the preparation can be tableted, placed in a hard gelatin capsule in powder or pellet form or in the form of a troche or lozenge. The amount of solid carrier will vary widely but preferably will be from about 25 mg. to about 1 g. When a liquid carrier is used, the preparation will be in the form of a syrup, emulsion, soft gelatin capsule, sterile injectable liquid such as an ampule or nonaqueous liquid suspension.

The present invention includes pharmaceutical compositions containing 0.1 to 99.5%, more particularly, 0.5 to 90% of a compound of the formula (I) in combination with a pharmaceutically acceptable carrier.

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

The compounds of the invention may be milled using known milling procedures such as wet milling to obtain a particle size appropriate for tablet formation and for other formulation types. Finely divided (nanoparticulate) preparations of the compounds of the invention may be prepared by processes known in the art, for example see WO 02/00196 (SmithKline Beecham).

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Thus, if a solid carrier is used, the preparation can be tableted, placed in a hard gelatin capsule in powder or pellet form or in the form of a troche or lozenge. The amount of solid carrier will vary widely but will generally be from about 25 mg. to about 1 g. When a liquid carrier is used, the preparation will be in the form of a syrup, emulsion, soft gelatin capsule, sterile injectable liquid such as an ampoule or nonaqueous liquid suspension.

For instance, for oral administration in the form of a tablet or capsule, the active drug component can be combined with an oral, non-toxic pharmaceutically acceptable inert carrier such as ethanol, glycerol, water and the like, or those as described above. The tablets may also contain excipients such as microcrystalline cellulose, lactose, sodium citrate, calcium carbonate, dibasic calcium phosphate and glycine, disintegrants such as starch (such as corn, wheat, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium or calcium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules can be made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an alginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acacia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously.

For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

For non-systemic administration this can include topical applications externally to the epidermis or the buccal cavity, or the instillation of such a compound into the ear, eye and nose, or inhalation into the lung, such that the compound does not significantly enter the blood stream, or mucosal (e.g. as a nasal spray or aerosol for inhalation)

In contrast, systemic administration refers to oral, parenteral (e.g. by injectable form, such as intravenous), intraperitoneal, intramuscular, and intraspinal administration. Other routes of administration can include, but are not limited to, intrauterine, intraocular, intradermal, intracranial, intratracheal, intravaginal, intracerebroventricular, intracerebral, subcutaneous, ophthalmic (including intravitreal or intracameral), transdermal, rectal, epidural and sublingual. It is to be understood that not all of the compounds need be administered by the same route. Likewise, if the composition comprises more than one active component, then those components may be administered by different routes.

The compounds of formula (I) and their pharmaceutically acceptable salts and solvates may be formulated for administration in any suitable manner. They may, for example, be formulated for topical administration or administration by inhalation or, more preferably, for oral, transdermal or parenteral administration. The pharmaceutical composition may be in a form such that it can affect controlled release of the compounds of formula (I) and their pharmaceutically acceptable derivatives.

In one embodiment of the present invention, the agents of the present invention are delivered via oral inhalation or intranasal administration. Appropriate dosage forms for such administration, such as an aerosol formulation or a metered dose inhaler, may be prepared by conventional techniques.

For administration by inhalation the compounds may be delivered in the form of an aerosol spray presentation from pressurized packs or a nebulizer, with the use of a suitable propellant, e.g. dichlorodifluoromethane, trichlorofluoromethane, dichlorotetrafluoroethane, a hydrofluoroalkane such as tetrafluoroethane or heptafluoropropane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of a compound of the invention and a suitable powder base such as lactose or starch.

Formulations suitable for topical administration include liquid or semi-liquid preparations suitable for penetration through the skin to the site of inflammation such as liniments, lotions, creams, ointments or pastes, and drops suitable for administration to the eye, ear or nose. The active ingredient may comprise, for topical administration, from about 0.001% to about 10% w/w, for instance from about 1% to about 2% by weight of the formulation. It may however comprise as much as about 10% w/w but generally may comprise less than 5% w/w, or in another embodiment from about 0.1% to about 1% w/w of the formulation.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as stearic or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops according to the present invention may comprise sterile aqueous or oily solutions or suspensions and may be prepared by dissolving the active ingredient in a suitable aqueous solution of a bactericidal and/or fungicidal agent and/or any other suitable preservative, and may include a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

For all methods of use disclosed herein for the compounds of Formula (I), the daily oral dosage regimen will in one embodiment be from about 0.1 to about 80 mg/kg of total body weight; or from about 0.2 to 30 mg/kg, or from about 0.5 mg to 15 mg. The daily parenteral dosage regimen may be from about 0.1 to about 80 mg/kg of total body weight, or from about 0.2 to about 30 mg/kg, or from about 0.5 mg to 15 mg/kg. The daily topical dosage regimen in one embodiment may be from 0.1 mg to 150 mg, administered one to four, suitably from two or three times daily. The daily inhalation dosage regimen will suitably be from about 0.001 mg/kg to about 1 mg/kg per day. It will also be recognized by one of skill in the art that the optimal quantity and spacing of individual dosages of a compound of Formula (I) or a pharmaceutically acceptable salt thereof will be determined by the nature and extent of the condition being treated, the form, route and site of administration, and the particular patient being treated, and that such optimums can be determined by conventional techniques. It will also be appreciated by one of skill in the art that the optimal course of treatment, i.e., the number of doses of a compound of Formula (I) or a pharmaceutically acceptable salt thereof given per day for a defined number of days, can be ascertained by those skilled in the art using conventional course of treatment determination tests.

The novel compounds of Formula (I) may also be used in association with the veterinary treatment of mammals, other than humans, in need of inhibition of CSBP/p38 or cytokine inhibition or production. In particular, CSBP/p38 mediated diseases for treatment, therapeutically or prophylactically, in animals include disease states such as those noted herein in the Methods of Treatment section. Examples of suitable viruses for treatment include, but are not limited to, lentivirus infections such as, equine infectious anaemia virus, caprine arthritis virus, visna virus, or maedi virus or retrovirus infections, such as but not limited to feline immunodeficiency virus (FIV), bovine immunodeficiency virus, or canine immunodeficiency virus or other retroviral infections.

Alternatively, the compound of the present invention can be administered in the form of a suppository or pessary, or it may be applied topically in the form of a gel, hydrogel, lotion, solution, cream, ointment or dusting powder.

The compounds of the present invention may also be administered by the pulmonary or rectal routes. They may also be administered by the ocular route. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

The pharmaceutical compositions generally are administered in an amount effective for treatment or prophylaxis of a specific condition or conditions. Initial dosing in humans is accompanied by clinical monitoring of symptoms, such symptoms for the selected condition.

### BIOLOGICAL EXAMPLES

The activity of compounds of formula (I) as p38 inhibitors may be determined by the following in vitro assays:

Fluorescence Anisotropy Kinase Binding Assay

The kinase enzyme, fluorescent ligand and a variable concentration of test compound are incubated together to reach thermodynamic equilibrium under conditions such that in the absence of test compound the fluorescent ligand is significantly (>50%) enzyme bound and in the presence of a sufficient concentration (>10×Kᵢ) of a potent inhibitor the anisotropy of the unbound fluorescent ligand is measurably different from the bound value.

The concentration of kinase enzyme should preferably be ≧1×K_{f}. The concentration of fluorescent ligand required will depend on the instrumentation used, and the fluorescent and physicochemical properties. The concentration used must be lower than the concentration of kinase enzyme, and preferably less than half the kinase enzyme concentration. A typical protocol is:

All components dissolved in Buffer of final composition 62.5 mM HEPES, pH 7.5, 1.25 mM CHAPS, 1.25 mM DTT, 12.5 mM MgCl₂ 3.3% DMSO.

p38 Enzyme concentration: 12 nM

Fluorescent ligand concentration: 5 nM

Test compound concentration: 0.1 nM-100 uM

Components incubated in 30 ul final volume in NUNC 384 well black microtitre plate until equilibrium reached (5-30 mins)

Fluorescence anisotropy read in LJL Acquest.

Definitions: Kᵢ=dissociation constant for inhibitor binding

K_{f}=dissociation constant for fluorescent ligand binding

The fluorescent ligand is the following compound: 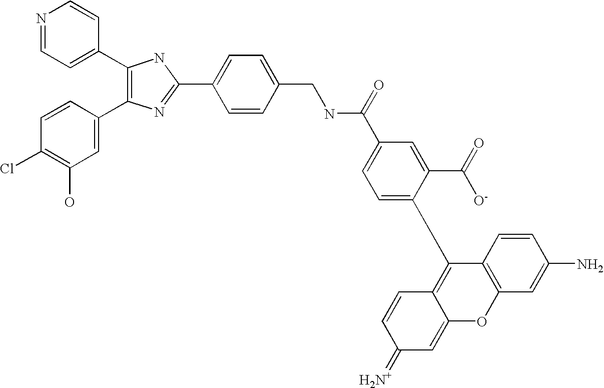
which is derived from 5-[2-(4-aminomethylphenyl)-5-pyridin-4-yl-1H-imidazol-4-yl]-2-chlorophenol and rhodamine green.
Results

Representative compounds of Formula (I), as described in the Example 22, and 79 were tested in the above assay and demonstrated pIC₅₀ value of 5.5, and 6.9 in this assay respectively.

Representative compounds of Formula (I) as described in Examples 10, 12, 13, and 25 demonstrated a pIC₅₀ value of less than 5.8 in this assay; and Examples, 75, 78 and 86 demonstrated a pIC₅₀ value of less than 4.8 in this assay.

HTRF Assay

Time-Resolved Fluorescence Resonance Energy Transfer Kinase Assay

Recombinant human p38α was expressed as a His-tagged protein. To activate this protein, 3 μM unactivated p38α was incubated in 200 mM Hepes pH7.4, 625 mM NaCl, 1 mM DTT with 27 nM active MKK6 (Upstate), 1 mM ATP and 10 mM MgCl₂. The activity of the MKK6-activated p38α was assessed using a time-resolved fluorescence resonance energy transfer (TR-FRET) assay.

Biotinylated-GST-ATF2 (residues 19-96, 400 nM final), ATP (125M final) and MgCl2 (5 mM final) in assay buffer (40 mM HEPES pH 7.4, 1 mM DTT) were added to wells containing 1 l of various concentrations of compound or DMSO vehicle (3% final) in NUNC 384 well black plate. The reaction was initiated by the addition of MKK6-activated p38 (100 pM final) to give a total volume of 30 μl. Alternatively, various concentrations of compound were incubated with activated p38 for up to 90 min, followed by initiation with the addition of ATF2, ATP and MgCl2. The reaction was incubated for 120 minutes at room temperature, then terminated by the addition of 15 μl of 100 mM EDTA pH 7.4. Detection reagent (15 μl) in buffer (100 mM HEPES pH 7.4, 150 mM NaCl, 0.1% w/v BSA, 1 mM DTT) containing antiphosphothreonine-ATF2-71 polyclonal antibody (Cell Signalling Technology, Beverly Mass., USA) labelled with W-1024 europium chelate (Wallac OY, Turku, Finland), and APC-labelled streptavidin (Prozyme, San Leandro, Calif., USA) was added and the reaction was further incubated for 60 minutes at room temperature. The degree of phosphorylation of GST-ATF2 was measured using a Packard Discovery plate reader (Perkin-Elmer Life Sciences, Pangbourne, UK) as a ratio of specific 665 nm energy transfer signal to reference europium 620 nm signal. Differences in IC50 with or without preincubation of enzyme with compound was interpreted as time-dependence.

Results

Compounds are considered active in this assay if they demonstrate a pIC₅₀ of greater than 5.8 up to about a pIC₅₀ of up to 10.0.

Representative compounds of Formula (I) as described in Examples 1-9, 14-24, 26-74, 76, 77, 80-85, and 87-90 all demonstrated a pIC₅₀ value of greater than 5.8 to about 9.0 in this assay.

Representative compounds of Formula (I) as described in Examples 10, 12, 13, 25, and 75 demonstrated a pIC₅₀ value of less than 5.8 in this assay, and Example 86 less than 4.6 in this assay.

For purposes herein the HTRF assay and the Fluorescence anisotropy kinase binding assay:

| pIC₅₀ | IC₅₀ (nM) | IC₅₀ (uM) |
|---|---|---|
| 4.00 | 100,000.0 | 100 |
| 5.00 | 100,000.0 | 10 |
| 6.00 | 1,000.0 | 1 |
| 7.00 | 100.0 | 0.1 |
| 8.00 | 10.0 | 0.01 |
| 9.00 | 1.0 | 0.001 |
| 10.00 | 0.1 | 0.0001 |

### Examples

The invention will now be described by reference to the following examples which are merely illustrative and are not to be construed as a limitation of the scope of the present invention. All temperatures are given in degrees centigrade, all solvents are highest available purity and all reactions run under anhydrous conditions in an argon atmosphere unless otherwise indicated. Mass spectra were performed upon a VG Zab mass spectrometer using fast atom bombardment, unless otherwise indicated. ¹H-NMR (hereinafter “NMR”) spectra were recorded at 250 MHz using a Bruker AM 250 or Am 400 spectrometer. Multiplicities indicated are: s=singlet, d=doublet, t=triplet, q=quartet, m=multiplet and br indicates a broad signal. Sat. indicates a saturated solution, eq indicates the proportion of a molar equivalent of reagent relative to the principal reactant. Flash chromatography is run over Merck Silica gel 60 (230-400 mesh).

### Example 1

### N²-[(1S)-1-Cyclohexylethyl]-N³′-cyclopropyl-N⁴-(2,2-dimethylpropyl)-5′-fluoro-6′-methyl-2,3′,4-biphenyltricarboxamide 1a) 4-Bromo-N-(2,2-dimethylpropyl)-3-formylbenzamide

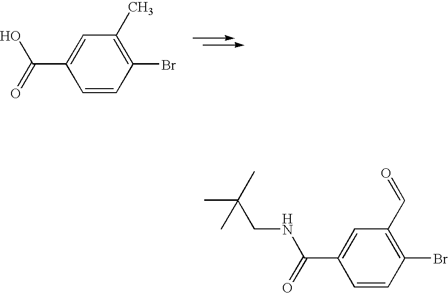

Bromine (14.8 ml, 0.286 mol) was added dropwise over 6 hour to a suspension of 4-bromo-3-methyl-benzoic acid (20 g, 0.093 mol) and dibenzoyl peroxide (0.25 g) in carbon tetrachloride (200 ml) at reflux, under nitrogen, while irradiating with a 250 W tungsten lamp. This was stirred at reflux for 4 days. The solvent was evaporated and the residue was triturated with toluene to give 4-bromo-3-(dibromomethyl)benzoic acid as an off-white solid (32.1 g).

4-Bromo-3-(dibromomethyl)benzoic acid (3.7 g, 10 mmol) was added to a solution of sodium carbonate (16 g, 56 mmol, 15 eq) in water (100 ml) then stirred at 70° C. overnight. The solution was brought to pH5 using 2M hydrochloric acid and the mixture was extracted with ethyl acetate. The organic extracts were dried and concentrated under vacuum to give 4-bromo-3-formylbenzoic acid as an off-white solid.

To a solution of 4-bromo-3-formylbenzoic acid (450 mg, 1.96 mmol) in DCM (20 mL) was added EDC (378 mg, 1.96 mmol), HOBt (27 mg, 0.20 mmol), Et₃N (1.10 mL, 7.84 mmol) and tert-butyl methylamine (230 uL, 1.96 mmol). The solution was stirred at room temperature over night. The reaction mixture was washed with H₂O (5 mL), dried over Na₂SO₄, filtered and concentrated. Flash chromatography then provided the title compound (346 mg, 59%). MS (ES) m/z 298 (M)⁺; ¹H-NMR (CDCl₃) δ 0.98 (s, 9H), 3.46 (s, 2H), 7.66 (d, J=1.2 Hz, 1H), 7.73 (m, 2H), 10.35 (s, 1H).

### 1b) N³-Cyclopropyl-N⁴′-(2,2-dimethylpropyl)-5-fluoro-2′-formyl-6-methyl-3,4′-biphenyldicarboxamide

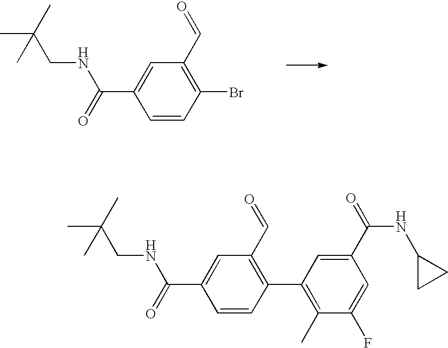

To a solution of 4-bromo-N-(2,2-dimethylpropyl)-3-formylbenzamide (340 mg, 1.14 mmol) in dioxane/H₂O (30/1.0 mL) was added {5-[(cyclopropylamino)carbonyl]-3-fluoro-2-methylphenyl}boronic acid (405 mg, 1.71 mmol), K₂CO₃ (788 mg, 5.70 mmol) and Pd(PPh₃)₄ (52.9 mL, 0.046 mmol). The mixture was heated with microwave to 150° C. for 15 minutes. The reaction mixture was filtered through celite, concentrated, extracted with EtOAc (2×30 mL), dried over Na₂SO₄, filtered and concentrated. Flash chromatography then provided the title compound (412 mg, 88%). MS (ES) m/z 411 (M+H)⁺; ¹H-NMR (CDCl₃) δ 0.63 (m, 2H), 0.82 (m, 2H), 0.99 (s, 9H), 2.03 (s, 3H), 2.90 (m, 1H), 3.33 (s, 2H), 7.37 (d, J=8.0 Hz, 1H), 7.42 (s, 2H), 7.58 (d, J=8.0 Hz, 1H), 8.15 (m, 1H), 8.27 (s, 1H), 9.72 (s, 1H).

### 1c) 5′-[(Cyclopropylamino)carbonyl]-4-{[(2,2-dimethylpropyl)amino]carbonyl}-3′-fluoro-2′-methyl-2-biphenylcarboxylic acid

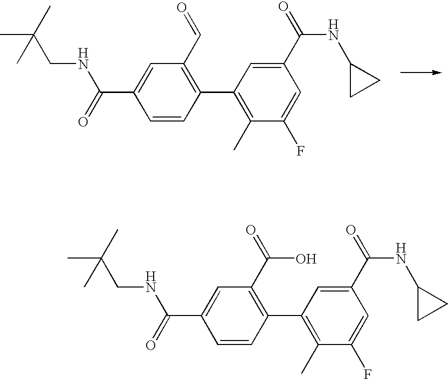

To a solution of N³-cyclopropyl-N⁴′-(2,2-dimethylpropyl)-5-fluoro-2′-formyl-6-methyl-3,4′-biphenyldicarbox amide (410 mg, 1.0 mmol) in acetone (10 mL) was added KMnO₄ (237 mg, 1.5 mmol) in H₂O (10 mL). The solution was stirred at room temperature for 2 hours. The reaction mixture was quenched with Na₂SO₃ (˜5 mL, 5%), filtered through celite, and mixed with AcOH (0.3 mL). The organic layer was separated and washed with brine, dried over Na₂SO₄, filtered and concentrated to afford the title compound (352 mg, 83%). MS (ES) m/z 427 (M+H)⁺.

### 1d) N²-[(1S)-1-Cyclohexylethyl]-N³′-cyclopropyl-N⁴-(2,2-dimethylpropyl)-5′-fluoro-6′-methyl-2,3′,4-biphenyltricarboxamide

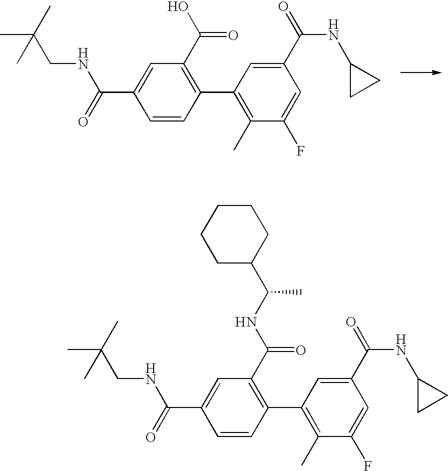 General Procedure I (Amide Formation)

To a solution of 5′-[(Cyclopropylamino)carbonyl]-4-{[(2,2-dimethylpropyl)-amino]carbonyl}-3′-fluoro-2′-methyl-2-biphenylcarboxylic acid (35.2 mg, 0.0825 mmol) in CH₂Cl₂ (2.0 mL) was added HBTU (34.4 mg, 0.908 mmol), Et₃N (23.3 uL, 0.165 mmol) and (1S)-1-cyclohexylethanamine (13.5 uL, 0.908 mmol). The solution was stirred at room temperature over night. The reaction mixture was quenched with H₂O (1 drop), mixed with DMSO (0.5 mL), concentrated and filtered. Purification via HPLC (Gilson) then afford the title compound (19 mg, 41%). MS (ES) m/z 536 (M+H)⁺; ¹H-NMR (CD₃OD) δ 0.63 (m, 2H), 0.81 (m, 4H), 1.01 (m, 12H), 1.14 (m, 4H), 1.60 (m, 5H), 2.10 (d, 3H), 3.84 (m, 1H), 3.27 (s, 2H), 3.68 (m, 1H), 7.39 (m, 1H), 7.55 (m, 1H), 7.92 (m, 1H), 8.00 (m, 2H).

### Example 2

### N³-Cyclopropyl-N⁴′-(2,2-dimethylpropyl)-5-fluoro-2′-[(4-hydroxy-1-piperidinyl)carbonyl]-6-methyl-3,4′-biphenyldicarboxamide

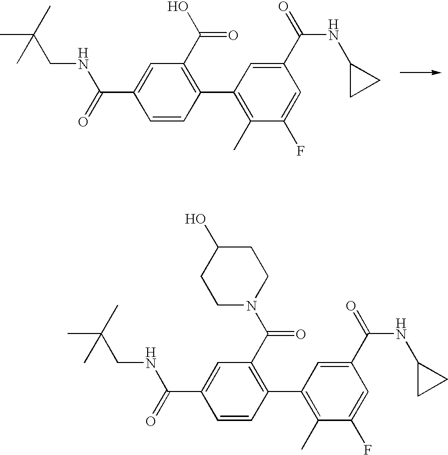

The title compound (23.7 mg, 56%) was prepared from 4-hydroxy-piperidine by following the general procedure 1. MS (ES) m/z 510 (M+H)⁺; ¹H-NMR (CD₃OD) δ 0.63 (m, 2H), 0.80 (m, 2H), 1.00 (s, 9H), 1.35 (m, 2H), 1.72 (m, 2H), 2.12 (m, 3H), 2.84 (m, 1H), 3.19 (m, 4H), 3.54 (m, 1H), 3.86 (m, 2H), 7.57 (m, 3H), 7.88 (m, 1H), 7.99 (m, 1H).

### Example 3

### N³′-Cyclopropyl-N⁴-(2,2-dimethylpropyl)-5′-fluoro-6′-methyl-N²-propyl-2,3′,4-biphenyltricarboxamide

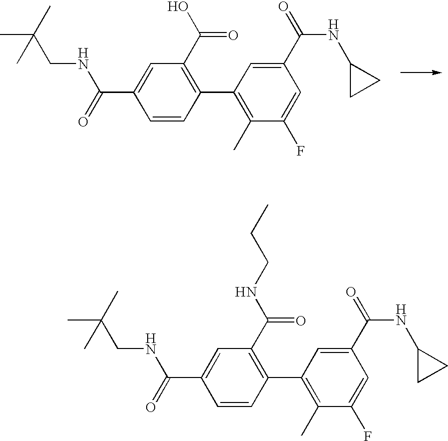

The title compound (11.9 mg, 31%) was prepared from n-propylamine by following the general procedure 1. MS (ES) m/z 468 (M+H)⁺; ¹H-NMR (CD₃OD) δ 0.63 (m, 2H), 0.80 (m, 5H), 1.01 (s, 9H), 1.36 (m, 2H), 2.09 (s, 3H), 2.84 (m, 1H), 3.11 (m, 4H), 3.27 (s, 2H), 7.41 (d, J=8.0 Hz, 1H), 7.49 (d, J=1.2 Hz, 1H), 7.53 (d, J=9.0 Hz, 1H), 8.00 (dd, J₁=8.0 Hz, J₂=2.0 Hz, 1H), 8.03 (d, J=2.0 Hz, 1H).

### Example 4

### N³′-Cyclopropyl-N²-[2-(dimethylamino)ethyl]-N⁴-(2,2-dimethylpropyl)-5′-fluoro-6′-methyl-2,3′,4-biphenyltricarboxamide

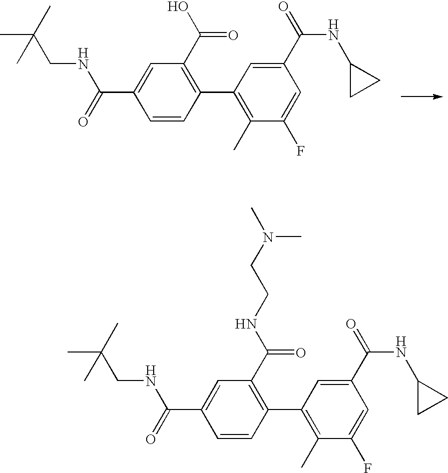

The title compound (8.9 mg, 22%) was prepared from 2-(dimethylamino)ethyl amine by following the general procedure 1. MS (ES) m/z 497 (M+H)⁺; ¹H-NMR (CD₃OD) δ 0.64 (m, 2H), 0.81 (m, 2H), 1.01 (s, 9H), 2.12 (d, J=2.0 Hz, 1H), 2.86 (s+m, 6+1H), 3.14 (t, J=6.4 Hz, 2H), 3.29 (s, 2H), 3.51 (m, 1H), 3.58 (m, 1H), 7.44 (d, J=8.0 Hz, 1H), 7.48 (d, J=1.2 Hz, 1H), 7.55 (dd, J₁=9.0 Hz, J₂=1.2 Hz, 1H), 8.03 (dd, J₁=8.0 Hz, J₂=2.0 Hz, 1H), 8.03 (d, J=2.0 Hz, 1H).

### Example 5

### N³′-Cyclopropyl-N⁴-(2,2-dimethylpropyl)-5′-fluoro-6′-methyl-N²-1,3-thiazol-2-yl-2,3′,4-biphenyltricarboxamide

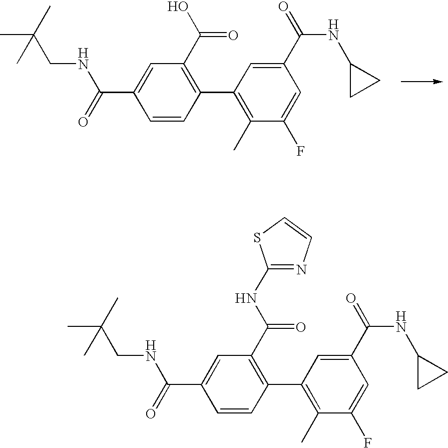

The title compound (10.3 mg, 25%) was prepared from 2-aminothiazole by following the general procedure 1. MS (ES) m/z 509 (M+H)⁺; ¹H-NMR (CD₃OD) δ 0.61 (m, 2H), 0.78 (m, 2H), 1.02 (s, 9H), 2.08 (s, 3H), 2.82 (m, 1 H), 3.30 (s, 2H), 7.11 (m, 1H), 7.40 (m, 1H), 7.50 (m, 3H), 8.11 (m, 1H), 8.25 (m, 1H).

### Example 6

### N³′-Cyclopropyl-N⁴-(2,2-dimethylpropyl)-5′-fluoro-6′-methyl-N²-(1-methylethyl)-2,3′,4-biphenyltricarboxamide

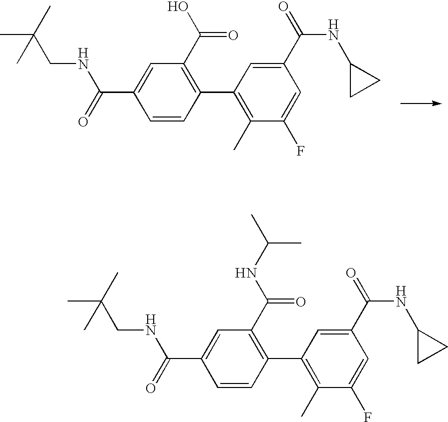

The title compound (23.3 mg, 60%) was prepared from 2-aminopropane by following the general procedure 1. MS (ES) m/z 468 (M+H)⁺; ¹H-NMR (CD₃OD) δ 0.63 (m, 2H), 0.82 (m, 2H), 0.97 (d, J=6.8 Hz, 3H), 1.01 (s, 9H), 1.05 (d, J=6.8 Hz, 3H), 2.09 (s, 3H), 2.84 (m, 1H), 3.28 (s, 2H), 3.91 (m, 1H), 7.42 (m, 1H), 7.51 (m, 1H), 7.58 (m, 1H), 7.66 (m, 1H), 8.00 (m, 2H).

### Example 7

N³-Cyclopropyl-N⁴′-(2,2-dimethylpropyl)-5-fluoro-6-methyl-2′-[(3-oxo-1-piperazinyl)carbonyl]-3,4′-biphenyldicarboxamide

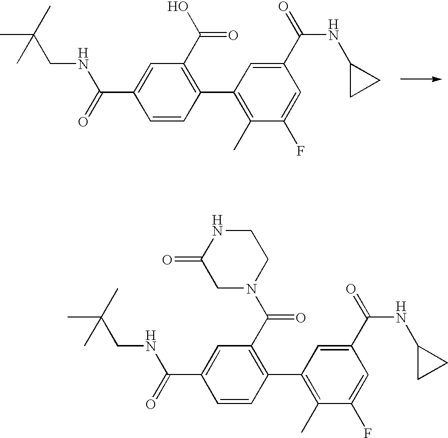

The title compound (23.6 mg, 56%) was prepared from 3-oxo-piperazine by following the general procedure 1. MS (ES) m/z 509 (M+H)⁺; ¹H-NMR (CD₃OD) δ 0.64 (m, 2H), 0.80 (m, 2H), 1.01 (s, 9H), 2.14 (s, 3H), 2.72 (br, 0.5 H), 2.85 (m, 1H), 3.15 (br, 1.5H), 3.28 (s, 2H), 3.51-4.16 (m, br, 4H), 7.50 (m, 1H), 7.58 (d, J=10.4 Hz, 1H), 7.94 (m, 1H), 8.03 (m, 1H), 8.58 (m, 1H).

### Example 8

N³′-Cyclopropyl-N⁴-(2,2-dimethylpropyl)-5′-fluoro-N²-[(2S)-2-hydroxypropyl]-6′-methyl-2,3′,4-biphenyltricarboxamide

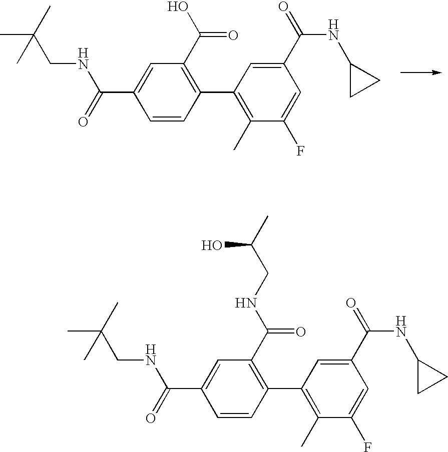

The title compound (11.9 mg, 30%) was prepared from (2S)-2-hydroxypropyl amine by following the general procedure 1. MS (ES) m/z 484 (M+H)⁺; ¹H-NMR (CD₃OD) δ 0.63 (m, 2H), 0.80 (m, 2H), 1.01 (s, 9H), 2.10 (s, 3H), 2.84 (m, 1H), 3.11 (m, 1H), 3.21 (m, 1H), 3.28 (s, 2H), 3.64 (m, 1H), 7.40 (d, J=8.0 Hz, 1H), 7.48 (s, 1H), 7.53 (d, J=10.4 Hz, 1H), 8.00 (dd, J₁=8.0 Hz, J₂=1.2 Hz, 1H), 8.07 (s, 1H).

### Example 9

### N³′-Cyclopropyl-N⁴-(2,2-dimethylpropyl)-5′-fluoro-N²-[(2R)-2-hydroxypropyl]-6′-methyl-2,3′,4-biphenyltricarboxamide

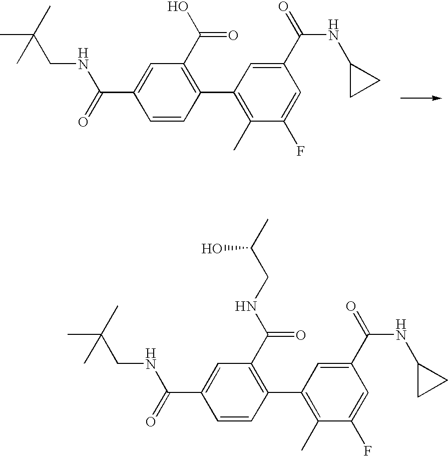

The title compound (13.6 mg, 34%) was prepared from (2R)-2-hydroxypropyl amine by following the general procedure 1. MS (ES) m/z 484 (M+H)⁺; ¹H-NMR (CD₃OD) δ 0.63 (m, 2H), 0.80 (m, 2H), 1.01 (s, 9H), 2.10 (s, 3H), 2.84 (m, 1H), 3.11 (m, 1H), 3.21 (m, 1H), 3.28 (s, 2H), 3.64 (m, 1H), 7.40 (d, J=8.0 Hz, 1H), 7.48 (s, 1H), 7.53 (d, J=10.4 Hz, 1H), 8.00 (dd, J₁=8.0 Hz, J₂=1.2 Hz, 1H), 8.07 (s, 1H).

### Example 10

### N⁴-(2,2-dimethylpropyl)-5′-fluoro-6′-methyl-N²-1,3-thiazol-2-yl-N³′-[(1R)-1,2,2-trimethylpropyl]-2,3′,4-biphenyltricarboxamide

### 10a) 2-bromo-5-{[(2,2-dimethylpropyl)amino]carbonyl}benzoic acid

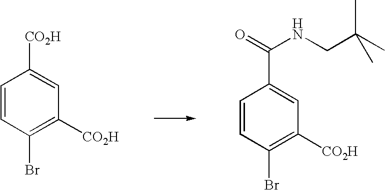

Triethylamine (2.8 mL, 20.4 mmol) was added to a fine suspension of 4-bromoisophthalic acid (5.0 g, 20.4 mmol) in dichloromethane (150 mL, HPLC grade) and the resultant reaction mixture was stirred for 30 min, at which point the mixture was completely homogenous. N,N-Carbonyldiimidazole (5.5 g, 33.7 mmol) was added portionwise over 5 min, the mixture was stirred for 1 h, and then neopentylamine (2.4 mL, 20.4 mmol) was added. After being stirred for 2.5 h, the solvent was removed under vacuum to afford a light brown foam (7.29 g), which was subsequently taken up in THF (30 mL) and treated with 2 M aqueous sodium hydroxide (30 mL) for 10 min. The reaction mixture was extracted with ethyl acetate (3×30 mL), the aqueous phase was acidified (pH 2-3) with concentrated hydrochloric acid, and the resultant white precipitate was collected by filtration and dried in air at 50° C. to afford 2-bromo-5-{[(2,2-dimethylpropyl)amino]carbonyl}benzoic acid (6.87 g, 100%), m.p. 176-179° C.

### 10b) methyl 2-bromo-5-{[(2,2-dimethylpropyl)amino]carbonyl}benzoate

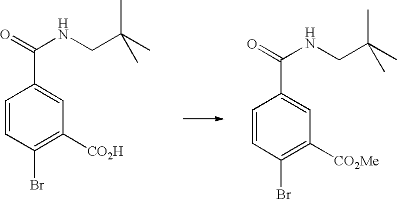

1-(3-Dimethylaminopropyl)-3-ethylcarbodiimide hydrochloride (14.2 g, 74.0 mmol) was added portionwise over 5 min to a stirred suspension of 2-bromo-5-{[(2,2-dimethylpropyl)-amino]carbonyl}benzoic acid (9.30 g, 29.6 mmol), 4-dimethylaminopyridine (996 mg, 0.89 mmol), and methanol (3.0 mL, 74.0 mmol) in dichloromethane (120 mL, HPLC grade) under an atmosphere of argon, and the resultant reaction mixture was stirred at room temperature for 16 h. Water (100 mL) was then added, the organic layer was separated, washed with 2 M aqueous sodium hydroxide (2×100 mL), brine (100 mL), dried (MgSO₄), and the solvent was removed under vacuum to afford the crude product as a pale yellow oil (9.89 g). This was purified by flash chromatography (silica, hexane to 50% ethyl acetate in hexane) to afford methyl 2-bromo-5-{[(2,2-dimethylpropyl)amino]-carbonyl}benzoate (4.82 g, 47%) as a white semi-solid.

### 10c) 3-fluoro-5-iodo-4-methylbenzoic acid

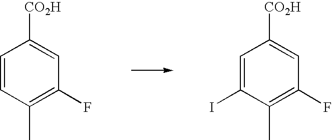

3-Fluoro-4-methyl benzoic acid (120 g, 0.78 mol) was added to triflic acid (830 mL) at 0° C. and the resultant mixture was cooled to −15° C. N-Iodosuccinimide (157 g, 0.70 mol) was then added in five portions over 30 min, while the temperature of the reaction mixture was kept below −10° C., and the resultant mixture was stirred at 0° C. for 3 h. A further portion of N-iodosuccinimide (58 g, 0.20 mol) was added and after being left to stand in the fridge for 24 h, a final portion (35 g, 0.16 mol) was added, and the reaction was left to stand in the fridge for a further 3 days. The reaction mixture was then poured into a mixture of ice (2.3 kg) and 10% aqueous sodium thiosulphate (1.2 L) and allowed to warm to room temperature. The resultant solid was collected by filtration, washed with water, air-dried, and taken up in ethyl acetate (4.0 L). The organic solution was then washed with 10% aqueous sodium thiosulphate (2×1.0 L) and saturated aqueous sodium chloride, and the aqueous phase was further extracted with ethyl acetate (2×1.0 L). The combined organic fractions were then dried (MgSO₄) and concentrated to a volume of about 320 mL, and the resultant solid was collected by filtration, washed with hexane, and air-dried to afford 3-fluoro-5-iodo-4-methylbenzoic acid (116 g, 53%) as an off-white solid.

### 10d) 1,1-dimethylethyl 3-fluoro-5-iodo-4-methylbenzoate

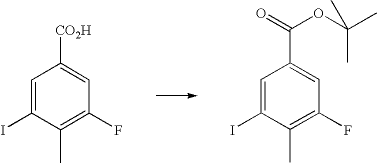

Oxalyl chloride (7.6 mL, 87.1 mmol) was added to a solution of 3-fluoro-5-iodo-4-methylbenzoic acid (20 g, 71.4 mmol) in tetrahydrofuran (100 mL) and a drop of N,N-dimethylformamide at 0° C., and the resultant reaction mixture was allowed to warm to room temperature over 1-2 h. The excess oxalyl chloride was removed under reduced pressure, tetrahydrofuran (100 mL) was added to the residue, the resultant solution was cooled to 0° C., and potassium tert-butoxide (8.0 g, 71.3 mmol) was added. The reaction mixture was then stirred at room temperature for 1 h, the majority of the solvent was removed under reduced pressure, and the residue was partitioned between ethyl acetate and water. The organic layer was separated, dried (MgSO₄), and the solvent was removed under reduced pressure. The crude product was purified by flash chromatography (silica, 5-20% ethyl acetate in hexane) to afford the title compound (16.6 g, 69%) as an off-white solid, m.p. 77-80° C.

### 10e) 1,1-dimethylethyl 3-fluoro-4-methyl-5-(4,4,5,5-tetramethyl-1,3,2-dioxaborolan-2-yl)benzoate

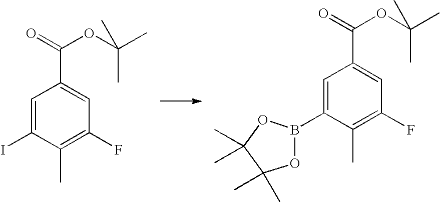

A mixture of 1,1-dimethylethyl 3-fluoro-5-iodo-4-methylbenzoate (15.0 g, 44.6 mmol), palladium(ii) chloride (0.38 g, 2.14 mmol), 1,1′-bis(diphenylphosphino)ferrocene (1.2 g, 2.16 mmol), and potassium acetate (13.2 g, 134.5 mmol) in N,N-dimethylformamide (200 mL) was stirred overnight under an atmosphere of argon, and was then poured into a mixture of ice/water and toluene. The resultant slurry was filtered through a pad of celite, which was washed with toluene, the organic layer was separated, and the solvent was removed under reduced pressure. The crude product was purified by flash chromatography (silica, 20% ethyl acetate in hexane) to afford the title compound (12.0 g, 80%) as pale yellow crystals, m.p. 64-68° C.

### 10f) 3′-(1,1-dimethylethyl) 2-methyl 4-{[(2,2-dimethylpropyl)amino]carbonyl}-5′-fluoro-6′-methyl-2,3′-biphenyldicarboxylate

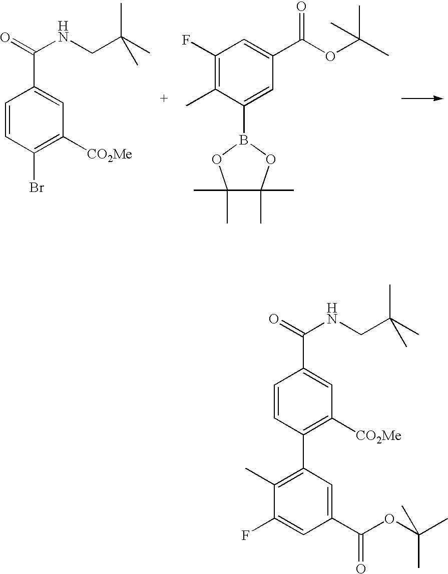

A solution of 1,1-dimethylethyl 3-fluoro-4-methyl-5-(4,4,5,5-tetramethyl-1,3,2-dioxaborolan-2-yl)benzoate (7.8 g, 21.4 mmol) in 1,4-dioxane (85 mL) was added to a stirred mixture of methyl 2-bromo-5-{[(2,2-dimethylpropyl)amino]carbonyl}benzoate (4.6 g, 13.4 mmol), tetrakis(triphenylphosphine)palladium(0) (3.1 g, 2.7 mmol), and 2 M aqueous sodium carbonate (6.7 mL, 13.4 mmol) in 1,4-dioxane (55 mL). Argon was bubbled through the solution for 30 min, the apparatus was flushed with argon, and the mixture was heated at reflux for 16 h and then allowed to cool to room temperature. Dichloromethane (200 mL) and water (250 mL) were added to the orange suspension and the aqueous layer was further extracted with dichloromethane (3×200 mL). The combined organic extracts were dried (MgSO₄) and concentrated under vacuum to afford a dark orange syrup (10.02 g). Purification of the crude product by flash column chromatography (silica, 10-40% ethyl acetate in hexane) afforded the title compound (5.84 g; 96%) as a yellow foam, m.p. 68-74° C.

### 10g) 5′-{[(1,1-dimethylethyl)oxy]carbonyl}-4-{[(2,2-dimethylpropyl)amino]-carbonyl}-3′-fluoro-2′-methyl-2-biphenylcarboxylic acid

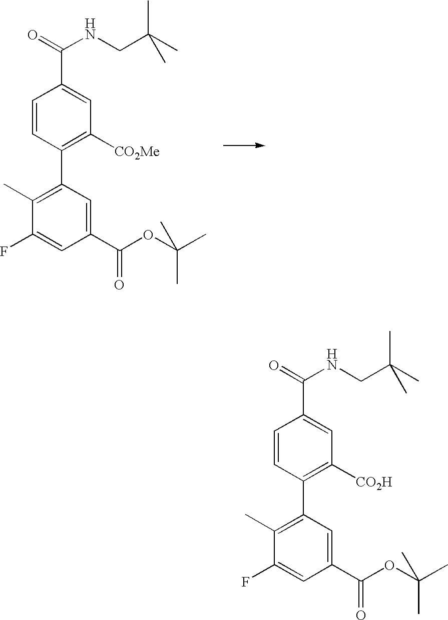

Lithium hydroxide monohydrate (2.44 g, 58.3 mmol) was added portionwise, over 5 min, to a stirred solution of 3′-(1,1-dimethylethyl) 2-methyl 4-{[(2,2-dimethylpropyl)amino]carbonyl}-5′-fluoro-6′-methyl-2,3′-biphenyldicarboxylate (5.33 g, 11.7 mmol) in tetrahydrofuran (50 mL) at 0° C., and the resultant reaction mixture was stirred at 0° C. for 5 min before water (25 mL) was added and the mixture was allowed to warm to room temperature. After stirring for 16 h, a further portion of lithium hydroxide monohydrate (1.22 g, 29.1 mmol) was added and stirring was continued for a further 24 h. The solvent was then removed under reduced pressure, the resultant residue was acidified to pH 3-4 with 1 M aqueous citric acid, and extracted with ethyl acetate (3×50 mL). The combined organic extracts were dried (MgSO₄) and concentrated under vacuum to afford the title compound (3.89 g, 75%) as a light brown glass, m.p. >240° C. (dec.).

### 10h) 1,1-dimethylethyl 4′-{[(2,2-dimethylpropyl)amino]carbonyl}-5-fluoro-6-methyl-2′-[(1,3-thiazol-2-ylamino)carbonyl]-3-biphenylcarboxylate

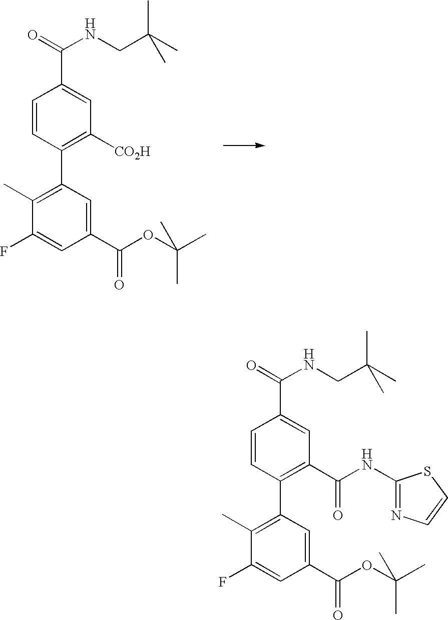

Benzotriazo-1-yloxytris(pyrrolidino)phosphonium (4.0 g, 7.69 mmol) was added portionwise, over 5 min, to a stirred solution of 5′-{[(1,1-dimethylethyl)oxy]carbonyl}-4-{[(2,2-dimethylpropyl)amino]carbonyl}-3′-fluoro-2′-methyl-2-biphenylcarboxylic acid (1.5 g, 3.4 mmol), 2-aminothiazole (406 mg, 4.1 mmol), and Hünigs base (1.6 mL, 10.2 mmol) in anhydrous N,N-dimethylformamide (23 mL). After being stirred for 16 h at room temperature, the reaction mixture was poured into water (50 mL), and the resultant off-white precipitate was collected by filtration. The solid was then taken up in ethyl acetate (20 mL), dried (MgSO₄), and concentrated under vacuum to afford a brown foam (1.87 g). Purification of the crude product by flash column chromatography (silica, 20-50% ethyl acetate in hexane with a few drops of triethylamine) afforded the title compound (1.31 g, 74%) as a light brown glass.

### 10i) 4′-{[(2,2-dimethylpropyl)amino]carbonyl}-5-fluoro-6-methyl-2′-[(1,3-thiazol-2-ylamino)carbonyl]-3-biphenylcarboxylic acid

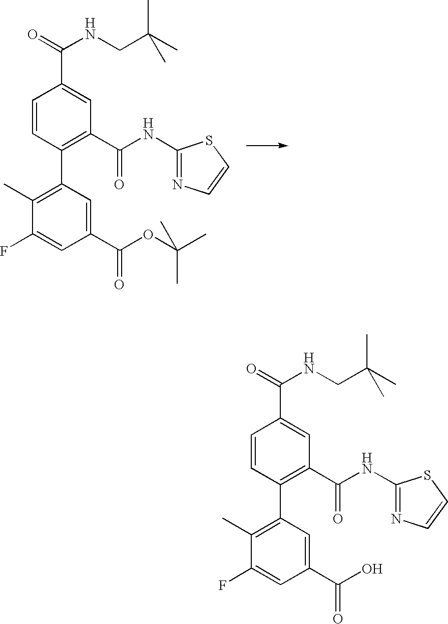

Aqueous hydrochloric acid (10 mL of a 20% solution) was added to a stirred solution of 1,1-dimethylethyl 4′-{[(2,2-dimethylpropyl)amino]carbonyl}-5-fluoro-6-methyl-2′-[(1,3-thiazol-2-ylamino)carbonyl]-3-biphenylcarboxylate (1.41 g, 2.68 mmol) in 1,4-dioxane (10 mL), and the resultant cloudy mixture was heated at reflux for 30 min. The mixture was then poured into water (50 mL) to form a ‘milky suspension’, which was extracted with ethyl acetate (3×50 mL). The combined organic extracts were dried (MgSO₄) and concentrated under vacuum to afford a pale brown foam (2.54 g). This was taken up in tetrahydrofuran (20 mL) and treated with 2 M aqueous sodium hydroxide (20 mL) for 10 min. after which time the mixture was washed with dichloromethane (3×10 mL). The aqueous phase was acidified to pH 1 with concentrated hydrochloric acid and the resultant gummy white suspension was extracted with ethyl acetate (3×30 mL). The organic extracts were dried (MgSO₄) and concentrated under vacuum to afford 4′-{[(2,2-dimethylpropyl)amino]carbonyl}-5-fluoro-6-methyl-2′-[(1,3-thiazol-2-ylamino)-carbonyl]biphenyl-3-carboxylic acid (1.26 g; 100%) as a white solid, m.p. 196-200° C.

### 10j) General Procedure for Amide Formation with 4′-{[(2,2-dimethylpropyl)amino]carbonyl}-5-fluoro-6-methyl-2′-[(1,3-thiazol-2-ylamino)-carbonyl]-3-biphenylcarboxylic acid

The requisite amine (0.26 mmol, 1.2 equiv.) was added in one portion to a stirred solution of 4′-{[(2,2-dimethylpropyl)amino]carbony}-5-fluoro-6-methyl-2′-[(1,3-thiazol-2-ylamino)carbonyl]biphen-yl-3-carboxylic acid (100 mg, 0.21 mmol), 1-(3-dimethylaminopropyl)-3-ethylcarbodiimide hydrochloride (98 mg, 0.51 mmol), and 4-dimethylaminopyridine (4.8 mg, 0.04 mmol) in anhydrous dichloromethane (840 μL) under an atmosphere of argon, and the resultant reaction mixture was stirred at room temperature for 16 h before being quenched with water (5 mL).

Work Up A

The reaction mixture was extracted with dichloromethane (3×5 mL) and the combined organic extracts were dried (MgSO₄) and concentrated under vacuum.

Work Up B

Ethyl acetate (2.0 mL) was added to the reaction mixture and the aqueous layer was further washed with ethyl acetate (3×2.0 mL). The aqueous phase was separated and concentrated to about 1 mL, cooled, and the resultant precipitate was collected by filtration and dried in air at 50° C.

Work Up C

Ethyl acetate (2.0 mL) was added to the reaction mixture and the aqueous layer was further extracted with ethyl acetate (3×2.0 mL). The combined organic extracts were dried (MgSO₄) and concentrated under vacuum to afford the crude product.

### 10k) N⁴-(2,2-dimethylpropyl)-5′-fluoro-6′-methyl-N²-1,3-thiazol-2-yl-N³′-[(1R)-1,2,2-trimethylpropyl]-2,3′,4-biphenyltricarboxamide

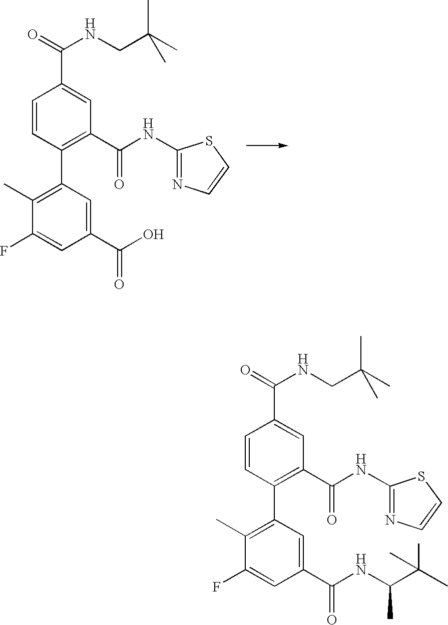

Use of (R)-(−)-3,3-dimethyl-2-butylamine, the above general procedure 10j, and work up A afford a light brown foam (137 mg). Purification of the crude product by flash chromatography (silica, 100% hexane to 60% ethyl acetate in hexane) afforded the title compound (65.5 mg, 56%) as an off-white solid, m.p. >125° C. (dec.). LC-MS m/z 553 (M+H)⁺

### Example 11

### 2′-amino-N³-cyclopropyl-N⁴′-(2,2-dimethylpropyl)-5-fluoro-6-methyl-3,4′-biphenyldicarboxamide

### 11a) phenylmethyl (2-bromo-5-{[(2,2-dimethylpropyl)amino]carbonyl}phenyl)carbamate

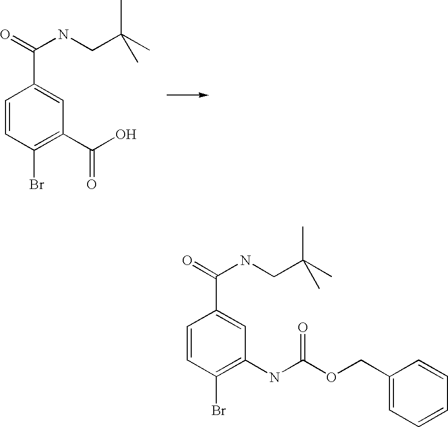

To a solution of 2-bromo-5-{[(2,2-dimethylpropyl)amino]carbonyl}benzoic acid (314 mg, 1.0 mmol) (example 10a) in toluene (3.0 mL) was added DPPA (216 uL, 1.0 mmol) and Et₃N (0.281 mL, 2.0 mmol). The solution was irradiated by microwave at 100° C. for 30 min. Benzyl alcohol (0.207 mL, 2.0 mmol) was added and the solution was stirred over night. The reaction mixture was concentrated and filtered. Purification via combiflash then afforded the title compound (343 mg, 82%). LC-MS m/z 420 (M+H)⁺.

### 11b) 2′-amino-N³-cyclopropyl-N⁴′-(2,2-dimethylpropyl)-5-fluoro-6-methyl-3,4′-biphenyldicarboxamide

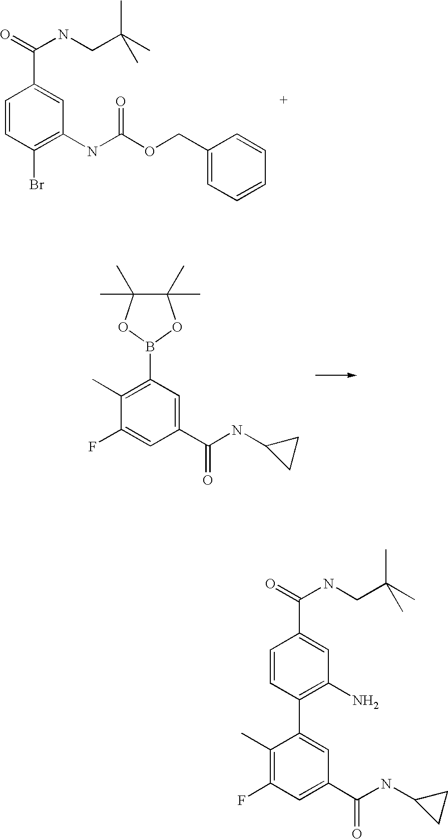

To a solution of phenylmethyl (2-bromo-5-{[(2,2-dimethylpropyl)amino]carbonyl}phenyl)carbamate (336 mg, 0.80 mmol) and N-cyclopropyl-3-fluoro-4-methyl-5-(4,4,5,5-tetramethyl-1,3,2-dioxaborolan-2-yl)benzamide (383 mg, 1.20 mmol) (example 28c) in H₂O (4.0 mL) and dioxane (12.0 mL) was added potassium carbonate (663 mg, 4.8 mmol) and Pd(PPh₃)₄ (46.4 mg, 0.04 mmol). The solution was microwaved at 150° C. for 30 min. The reaction mixture was concentrated and filtered. Purification via combiflash then afforded the title compound (167 mg, 52%). LC-MS m/z 398 (M+H)⁺.

### Example 12

### N³′-[(1S)-1,2-dimethylpropyl]-N⁴-(2,2-dimethylpropyl)-5′-fluoro-6′-methyl-N²-1,3-thiazol-2-yl-2,3′,4-biphenyltricarboxamide

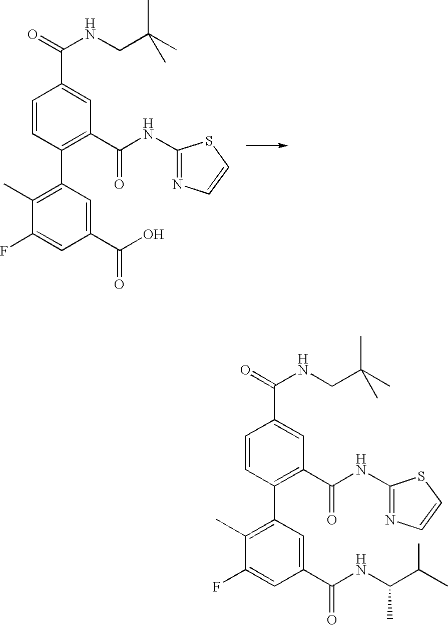

Use of (S)-(−)-3-methyl-2-butylamine, the above general procedure 10j, and work up C afforded an off-white foam (115 mg). The crude material was purified by flash chromatography (silica, 100% hexane to 60% ethyl acetate in hexane with a few drops of triethylamine) to afford the title compound (59.5 mg, 53%) as a white solid, m.p. 234-239° C. LC-MS m/z 539 (M+H)⁺.

### Example 13

### N³′-[(1R)-1,2-dimethylpropyl]-N₄-(2,2-dimethylpropyl)-5′-fluoro-6′-methyl-N²-1,3-thiazol-2-yl-2,3′,4-biphenyltricarboxamide

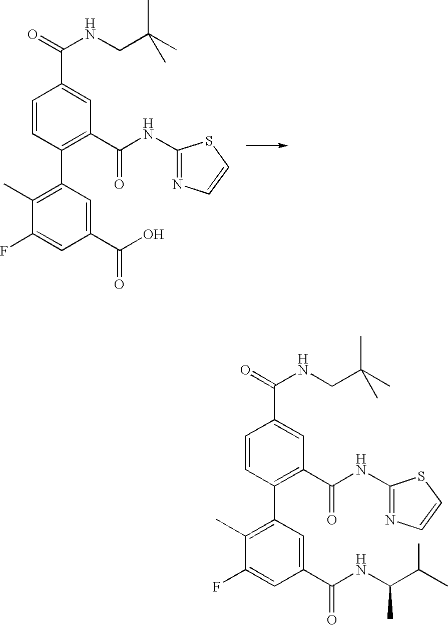

Use of (R)-(−)-3-methyl-2-butylamine, the above general procedure 10j, and work up C afforded a colourless oil (110 mg). The crude material was purified by flash chromatography (silica, 100% hexane to 60% ethyl acetate in hexane with a few drops of triethylamine) to afford the title compound (47.9 mg, 42%) as a white solid, m.p. 240-244° C. LC-MS m/z 539 (M+H)⁺.

### Example 14

### N⁴-(2,2-dimethylpropyl)-5′-fluoro-6′-methyl-N³′-(2-methylpropyl)-N²-1,3-thiazol-2-yl-2,3′,4-biphenyltricarboxamide

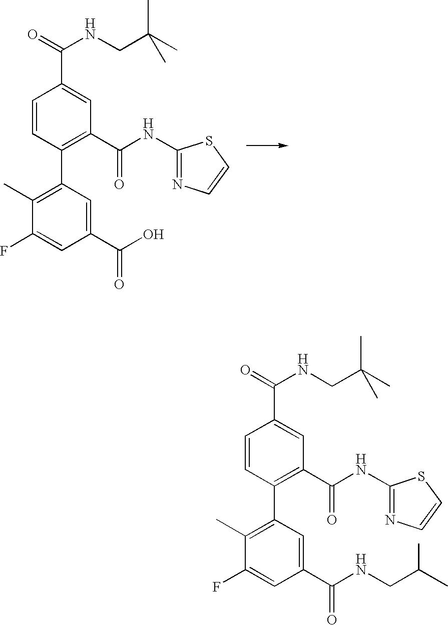

Use of isobutylamine, the above general procedure 10j, and work up C afforded an off-white foam (115 mg). The crude material was purified by flash chromatography (silica, 100% hexane to 60% ethyl acetate in hexane with a few drops of triethylamine) to afford the title compound (47.8 mg, 43%) as an off-white solid, m.p. >194° C. (dec.). LC-MS m/z 525 (M+H)⁺.

### Example 15

### N³′-(cyclopropylmethyl)-N⁴-(2,2-dimethylpropyl)-5′-fluoro-6′-methyl-N²-1,3-thiazol-2-yl-2,3′,4-biphenyltricarboxamide

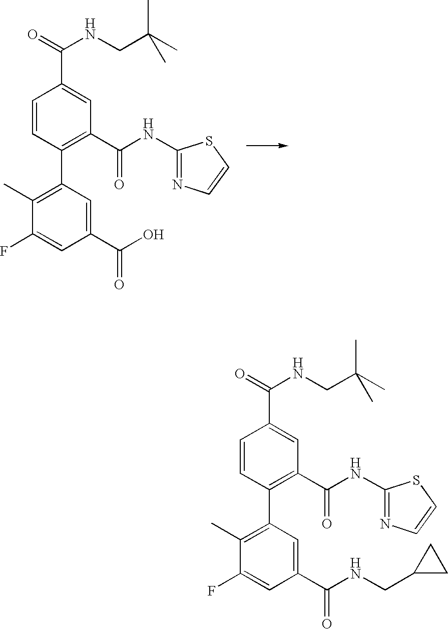

Use of cyclopropanemethylamine, the above general procedure 10j, and work up C afforded a yellow oil (79 mg). The crude material was purified by flash chromatography (silica, 100% hexane to 60% ethyl acetate in hexane with a few drops of triethylamine) to afford the title compound (37.4 mg, 34%) as an off-white solid, m.p. 179-183° C. LC-MS m/z 523 (M+H)⁺.

### Example 16

### N⁴-(2,2-dimethylpropyl)-5′-fluoro-6′-methyl-N³′-(3-methylbutyl)-N²-1,3-thiazol-2-yl-2,3′,4-biphenyltricarboxamide

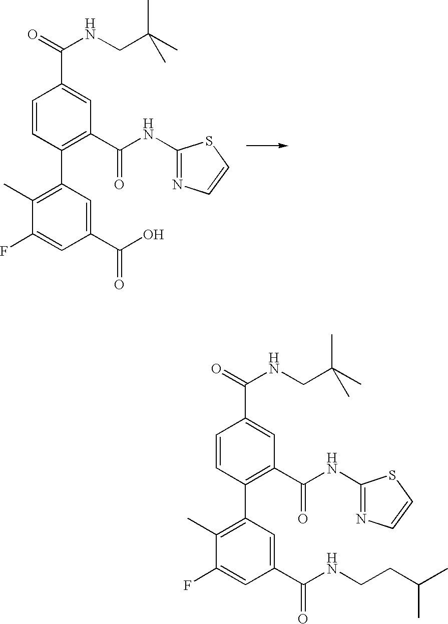

Use of isoamylamine, the above general procedure 10j, and work up C afforded a yellow oil (98 mg). The crude material was purified by flash chromatography (silica, 100% hexane to 60% ethyl acetate in hexane with a few drops of triethylamine) to afford the title compound (59.9 mg, 53%) as an off-white solid, m.p. 159-164° C. LC-MS m/z 539 (M+H)⁺.

### Example 17

### N⁴-(2,2-dimethylpropyl)-N³′-(3,5-dimethyl-1H-pyrazol-4-yl)-5′-fluoro-6′-methyl-N²-1,3-thiazol-2-yl-2,3′,4-biphenyltricarboxamide

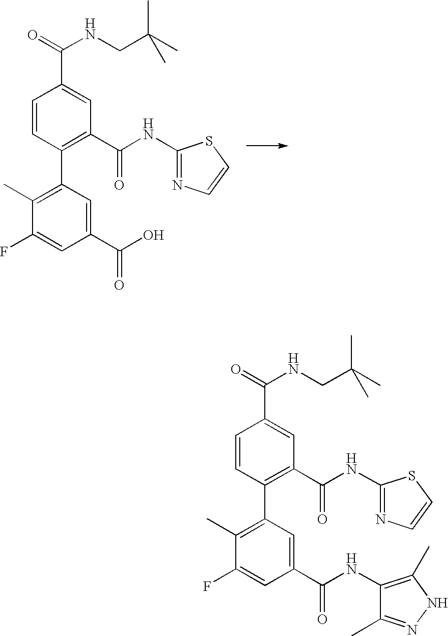

Use of 3,5-dimethyl-1H-pyrazol-4-ylamine, the above general procedure 10j, and work up C yielded crude product, which was purified by flash chromatography (silica, 1-10% methanol in dichloromethane) to afford the title compound as a pale brown solid (37 mg, 31%). LC-MS m/z 563 (M+H)⁺.

### Example 18

### N⁴-(2,2-dimethylpropyl)-5′-fluoro-6′-methyl-N³′-1,2,4-thiadiazol-5-yl-N²-1,3-thiazol-2-yl-2,3′,4-biphenyltricarboxamide

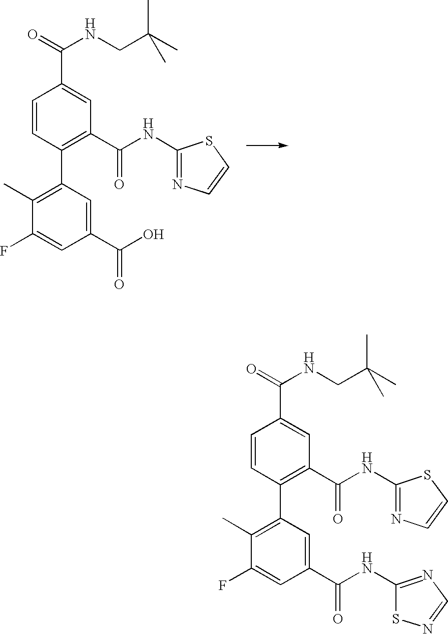

Use of [1,2,4]thiadiazol-5-ylamine, the above general procedure 10j, and work up C yielded crude product, which was purified by flash chromatography (silica, 2-10% methanol in dichloromethane) to afford the title compound as a white solid (64 mg, 54%). LC-MS m/z 553 (M+H)⁺.

### Example 19

### N⁴-(2,2-dimethylpropyl)-5′-fluoro-6′-methyl-N³′-1H-pyrazol-3-yl-N²-1,3-thiazol-2-yl-2,3′,4-biphenyltricarboxamide

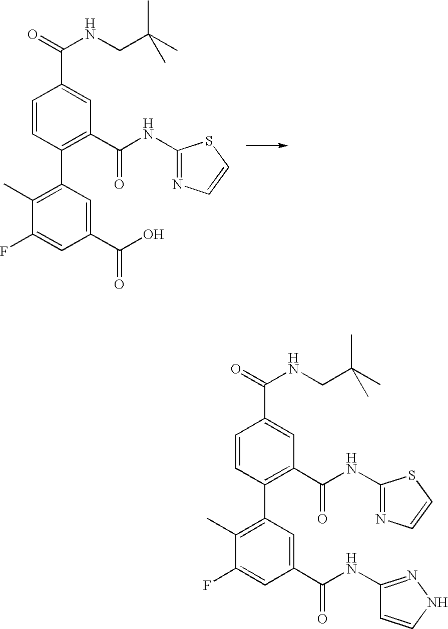

Use of 1H-pyrazol-3-ylamine, the above general procedure 10j, and work up C yielded crude product, which was purified by flash chromatography (silica, 2-10% methanol in dichloromethane) to afford the title compound (64 mg, 56%). LC-MS m/z 535 (M+H)⁺.

### Example 20

### N⁴-(2,2-dimethylpropyl)-5′-fluoro-6′-methyl-N³′-(3-methyl-5-isothiazolyl)-N²-1,3-thiazol-2-yl-2,3′,4-biphenyltricarboxamide

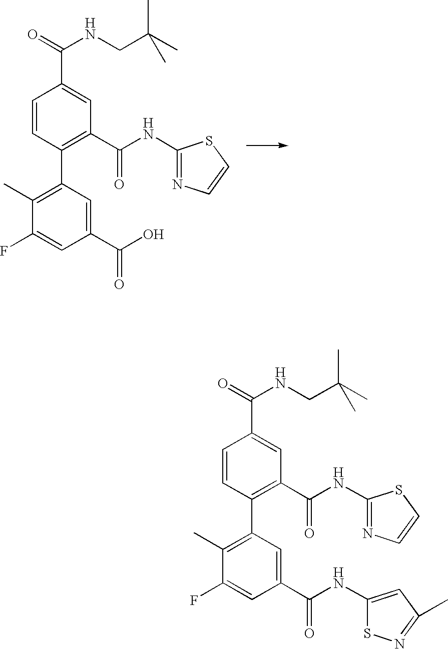

Use of 3-methylisothiazol-5-ylamine, the above general procedure 10j, and work up C yielded crude mixture, which was purified by flash chromatography (silica, 2-10% methanol in dichloromethane) to afford the title compound as a yellow solid (64 mg, 53%). LC-MS m/z 566 (M+H)⁺.

### Example 21

### N⁴-(2,2-dimethylpropyl)-5′-fluoro-N³′-3-isoxazolyl-6′-methyl-N²-1,3-thiazol-2-yl-2,3′,4-biphenyltricarboxamide

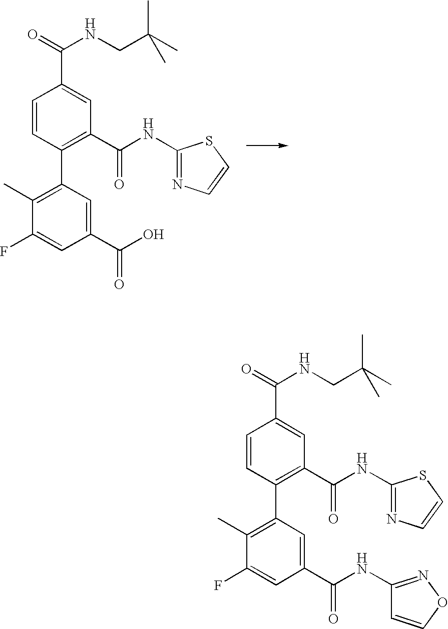

Use of isoxazol-3-ylamine, the above general procedure 10j, and work up C yielded crude product, which was purified by flash chromatography (silica, 2-10% methanol in dichloromethane) to afford the title compound as a yellow solid (40 mg, 35%). LC-MS m/z 536 (M+H)⁺.

### Example 22

### N³′-cyclohexyl-N⁴-(2,2-dimethylpropyl)-5′-fluoro-6′-methyl-N²-1,3-thiazol-2-yl-2,3′,4-biphenyltricarboxamide

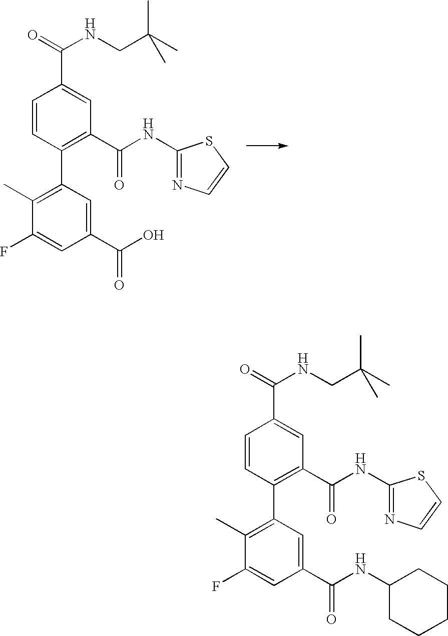

Cyclohexylamine (230 uL, 2.02 mmol) was added to a stirred solution of 4′-{[(2,2-dimethylpropyl)amino]carbony}-5-fluoro-6-methyl-2′-[(1,3-thiazol-2-ylamino)carbonyl]biphen-yl-3-carboxylic acid (100 mg, 0.21 mmol), 1-(3-dimethylaminopropyl)-3-ethylcarbodiimide hydrochloride (85 mg, 0.43 mmol), and 4-dimethylaminopyridine (10 mg, 0.082 mmol) in anhydrous dichloromethane (3.0 mL) under an atmosphere of argon, and the resultant reaction mixture was stirred at room temperature for 3 days before being quenched with water (10 mL). Work up C and purification of the crude product by flash chromatography (silica, 20-33% ethyl acetate in hexane) afforded the title compound (30 mg, 26%) as a white solid. LC-MS m/z 551 (M+H)⁺.

### Example 23

### N⁴-(2,2-dimethylpropyl)-5′-fluoro-6′-methyl-N²,N³′-di-1,3-thiazol-2-yl-2,3′,4-biphenyltricarboxamide

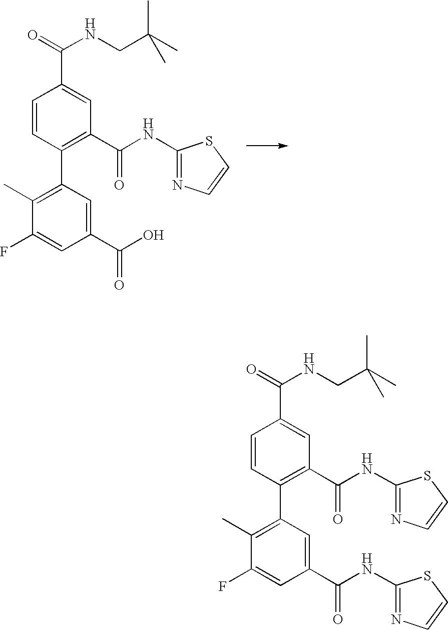

2-Aminothiazole (30 mg, 0.30 mmol) was added to a stirred solution of 4′-{[(2,2-dimethylpropyl)amino]carbony}-5-fluoro-6-methyl-2′-[(1,3-thiazol-2-ylamino)carbonyl]biphen-yl-3-carboxylic acid (100 mg, 0.21 mmol), 1-(3-dimethylaminopropyl)-3-ethylcarbodiimide hydrochloride (85 mg, 0.43 mmol), and 4-dimethylaminopyridine (10 mg, 0.082 mmol) in anhydrous dichloromethane (3.0 mL) under an atmosphere of argon, and the resultant reaction mixture was stirred at room temperature for 3 d before being quenched with water (10 mL). Work up C and purification of the crude product by flash chromatography (silica, 20-33% ethyl acetate in hexane) afforded the title compound (50 mg, 43%) as a white solid. LC-MS m/z 552 (M+H)⁺.

### Example 24

### N⁴-(2,2-dimethylpropyl)-5′-fluoro-6′-methyl-N³′-phenyl-N²-1,3-thiazol-2-yl-2,3′,4-biphenyltricarboxamide

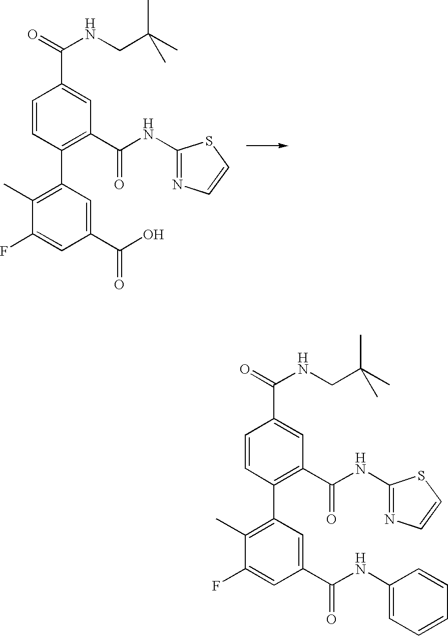

Aniline (49 μL, 0.54 mmol) was added to a stirred solution of 4′-{[(2,2-dimethylpropyl)amino]carbony}-5-fluoro-6-methyl-2′-[(1,3-thiazol-2-ylamino)carbonyl]biphen-yl-3-carboxylic acid (100 mg, 0.21 mmol), 1-(3-dimethylaminopropyl)-3-ethylcarbodiimide hydrochloride (85 mg, 0.43 mmol), and 4-dimethylaminopyridine (10 mg, 0.082 mmol) in anhydrous dichloromethane (3.0 mL) under an atmosphere of argon, and the resultant reaction mixture was stirred at room temperature for 3 d before being quenched with water (10 mL). Work up C and purification of the crude product by flash chromatography (silica, 20-33% ethyl acetate in hexane) afforded the title compound (40 mg, 35%) as a white solid. LC-MS m/z 545 (M+H)⁺.

### Example 25

### N⁴-(2,2-dimethylpropyl)-5′-fluoro-6′-methyl-N²-1,3-thiazol-2-yl-N³′-[(1S)-1,2,2-trimethylpropyl]-2,3′,4-biphenyltricarboxamide

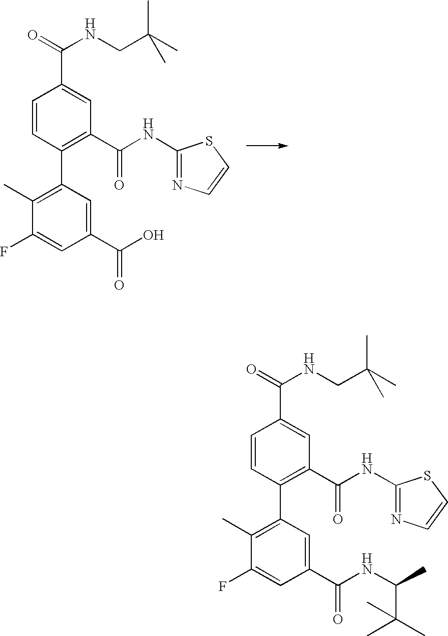

[(1S)-1,2,2-trimethylpropyl]amine (0.26 mmol, 1.2 equiv.) was added in one portion to a stirred solution of 4′-{[(2,2-dimethylpropyl)amino]carbonyl}-5-fluoro-6-methyl-2′-[(1,3-thiazol-2-ylamino)-carbonyl]-3-biphenylcarboxylic acid (100 mg), EDCI (100 mg) and DMAP (4 mg) in anhydrous dichloromethane (2 mL) under an atmosphere of argon, and the resultant reaction mixture was stirred at room temperature for 16 h. Work up C yielded crude product, which was purified by passing through a plug of silica to remove baseline impurities to afford the title compound (103 mg). mp 160-165° C.; LC-MS m/z 553 (M+H)⁺.

### Example 26

### N³′-cyclopropyl-N⁴,N⁴-diethyl-5′-fluoro-6′-methyl-N²-1,3-thiazol-2-yl-2,3′,4-biphenyltricarboxamide

### 26a) 3-fluoro-5-iodo-4-methylbenzoic acid

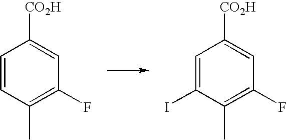

3-Fluoro-4-methyl benzoic acid (120 g, 0.78 mol) was added to triflic acid (830 mL) at 0° C. and the resultant mixture was cooled to −15° C. N-Iodosuccinimide (157 g, 0.70 mol) was then added in five portions over 30 min, while the temperature of the reaction mixture was kept below −10° C., and the resultant mixture was stirred at 0° C. for 3 h. A further portion of N-iodosuccinimide (58 g, 0.20 mol) was added and after being left to stand in the fridge for 24 h, a final portion (35 g, 0.16 mol) was added, and the reaction was left to stand in the fridge for a further 3 days. The reaction mixture was then poured into a mixture of ice (2.3 kg) and 10% aqueous sodium thiosulphate (1.2 L) and allowed to warm to room temperature. The resultant solid was collected by filtration, washed with water, air-dried, and taken up in ethyl acetate (4.0 L). The organic solution was then washed with 10% aqueous sodium thiosulphate (2×1.0 L) and saturated aqueous sodium chloride, and the aqueous phase was further extracted with ethyl acetate (2×1.0 L). The combined organic fractions were then dried (MgSO₄) and concentrated to a volume of about 320 mL, and the resultant solid was collected by filtration, washed with hexane, and air-dried to afford the title compound (116 g, 53%) as an off-white solid.

### 26b) N-cyclopropyl-3-fluoro-5-iodo-4-methylbenzamide

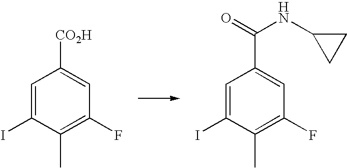

To a suspension of 3-fluoro-5-iodo-4-methylbenzoic acid (28 g, 100 mmol) in DCM (200 ml) cooled to 0° C. an ice bath, was slowly added oxalyl chloride (10.5 ml, 1.1 equivs). The solid dissolved and the resultant solution was stirred to ambient temperature over two hours and then concentrated. DCM (200 ml) was added to the residue and the resultant solution cooled in an ice bath. A solution of cyclopropylamine (5.7 g, 1 equiv) and triethylamine (20 ml) in DCM (50 ml) was added at 0° C. Once addition was complete, the mixture was stirred to RT over three hours and quenched by the addition of water (100 ml). Layers were separated and the organic washed with water 100 ml). The aqueous was re-extracted with ethyl acetate (50 ml) and the combined organics dried (MgSO₄) and concentrated. The residue was purified by flash chromatography (silica, 1:2 EtOAc:hexanes) to afford the title compound (18 g, 54%) as a pale yellow solid; mp=112-114° C.

### 26c) N-cyclopropyl-3-fluoro-4-methyl-5-(4,4,5,5-tetramethyl-1,3,2-dioxaborolan-2-yl)benzamide

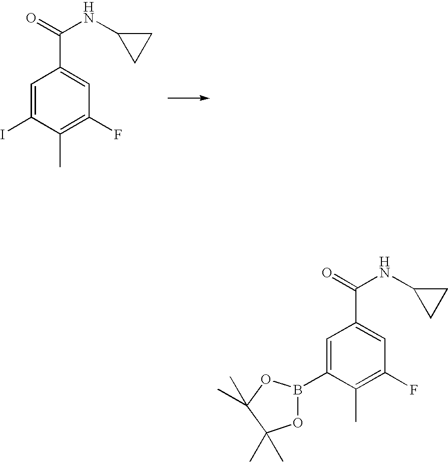

A mixture of N-cyclopropyl-3-fluoro-5-iodo-4-methylbenzamide (18 g, 53 mmol), palladium (ii) chloride (0.99 g, 5.6 mmol), 1,1′-bis(diphenylphosphino)-ferrocene (2.97 g, 5.4 mmol), and potassium acetate (18 g, 183.5 mmol) in N,N-dimethylformamide (200 mL) was stirred at 90° C. for four hours under an atmosphere of argon. Mixture was allowed to cool and was poured onto ice water. After filtering through a plug of celite, the product was extracted with toluene (2×150 ml). The toluene solution was washed with water (50 ml), dried (MgSO₄) and concentrated. The crude product was purified by flash chromatography (silica, 1:1 EtOAc:hexane) to give, after trituration with hexanes, the title compound (9.2 g, 52%), mp=191-192° C.

### 26d) methyl 4-bromo-3-methylbenzoate

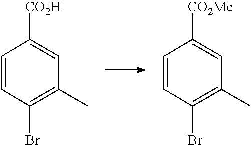

Acetyl chloride (20 mL) was added drop-wise to methanol (100 mL) during 20 min then the mixture stirred for 5 min. 4-Bromo-3-methylbenzoic acid (16.1 g, 75 mmol) was added and the mixture heated to reflux then stirred for 18 h. The solvent was evaporated and the residue dissolved in EtOAc (300 mL). This was washed with cold 0.5 M NaOH_{(aq)} (20 mL), water (10 mL) then dried (MgSO₄), filtered and evaporated under reduced pressure to give an oil which solidified on standing to give the title compound as a white solid (16.0 g, 93%), mpt: 38-41° C.

### 26e) methyl 4-bromo-3-(bromomethyl)benzoate

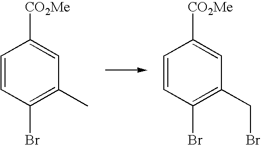

A solution of methyl 4-bromo-3-methylbenzoate (20.0 g, 87.3 mmol), N-bromosuccinimide (18.7 g, 105 mmol), 1,1′-azobis(cyclohexanecarbonitrile) (0.5 g) in acetonitrile (100 mL) was heated at reflux for 12 h. This was cooled to room temperature then the solvent evaporated under reduced pressure. The residue was dissolved in water (100 mL) and EtOAc (200 mL) then the organic layer separated, washed with water (2×100 mL), brine (100 mL) then dried (MgSO₄) filtered and evaporated under reduced pressure.

Recrystallisation from 10:1, hexane:EtOAc gave the title compound as a white solid (19.2 g, 72%); mpt: 88-92° C.

### 26f) methyl 4-bromo-3-(hydroxymethyl)benzoate

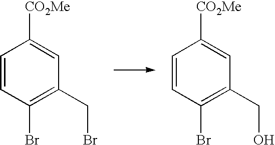

A mixture of methyl 4-bromo-3-(bromomethyl)benzoate (5.00 g, 16.2 mmol), wet DMSO (30 mL) and sodium bicarbonate (8.00 g, 95.2 mmol) were heated at 70° C. for 20 min. This was cooled in an ice-bath, added to water (50 mL) and extracted with EtOAc (3×30 mL). The organic extracts were washed with water (2×20 mL), brine (20 mL), dried (MgSO₄), filtered and evaporated under reduced pressure. Purification by flash chromatography (10:1→4:1, hexane:EtOAc) gave the title compound as a colourless oil (2.36 g, 60%).

26a) methyl 4-bromo-3-formylbenzoate

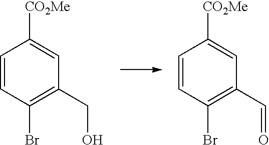

A mixture of methyl 4-bromo-3-(hydroxymethyl)benzoate (9.41 g, 38.4 mmol) and manganese dioxide (40 g) were heated at reflux for 18 h in dichloromethane (40 mL). Further portions of manganese dioxide (1.0 g) were added at hourly intervals until reaction was complete by TLC (ca. 5.0 g added). The mixture was filtered through celite, washing with EtOAc (500 mL) then the filtrate evaporated under reduced pressure to give the title compound as a pale yellow solid (7.8 g, 84%); mpt: 71-74° C.

### 26h) methyl 5′-[(cyclopropylamino)carbonyl]-3′-fluoro-2-formyl-2′-methyl-4-biphenylcarboxylate

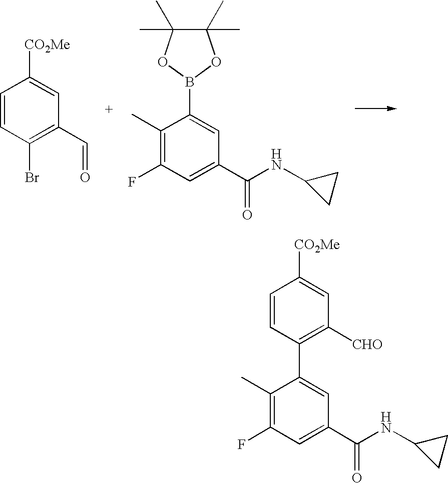

Dry DMF (200 mL) was de-gassed with argon for 30 min then methyl 4-bromo-3-formylbenzoate (7.0 g, 28.8 mmol), N-cyclopropyl-3-fluoro-4-methyl-5-(4,4,5,5-tetramethyl-1,3,2-dioxaborolan-2-yl)benzamide (9.2 g, 28.8 mmol), potassium carbonate (6.0 g, 43.5 mmol) and Pd(PPh₃)₄ (1.67 g, 1.44 mmol) added then the mixture heated at 90° C. for 7 h. This was stood overnight then poured onto ice-water (400 mL). This was extracted with EtOAc (2×300 mL) then the EtOAc extracts washed with water (200 mL), brine (100 mL) then dried (MgSO₄), filtered and evaporated under reduced pressure. Purification by flash chromatography (10:1→4:1→1:1, hexane:EtOAc) gave the title compound as a light brown solid (6.2 g, 61% contains a little DMF).

### 26i) 5′-[(cyclopropylamino)carbonyl]-3′-fluoro-2′-methyl-4-[(methyloxy)carbonyl]-2-biphenylcarboxylic acid

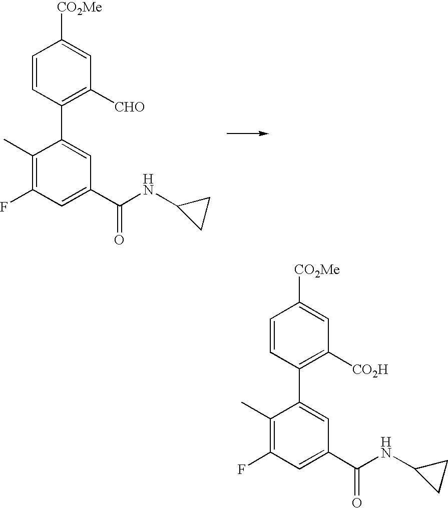

To a suspension of methyl 5′-[(cyclopropylamino)carbonyl]-3′-fluoro-2-formyl-2′-methyl-4-biphenylcarboxylate (6.20 g, 17.5 mmol), sodium chlorite (3.51 g, 38.8 mmol), potassium dihydrogenorthophosphate (8.5 g, 62.6 mmol) in acetonitrile (50 mL) was added a pre-mixed solution of 30% hydrogen peroxide (4.1 mL) and sodium sulphite (2.2 g, 17.5 mmol) in water (50 mL) at 0-5° C. This was allowed to warm to rt and stirred for 30 min. The acetonitrile was evaporated under reduced pressure and the mixture cooled to rt then the precipitate filtered off, washing with water (50 mL). This was dried in air to give the title compound as a pale brown solid (6.03 g, 93%); mpt: 217-222° C.

### 26j) methyl 5′-[(cyclopropylamino)carbonyl]-3′-fluoro-2′-methyl-2-[(1,3-thiazol-2-ylamino)carbonyl]-4-biphenylcarboxylate

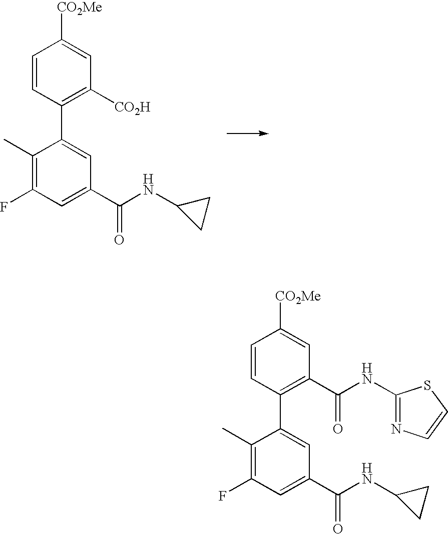

To a suspension of 5′-[(cyclopropylamino)carbonyl]-3′-fluoro-2′-methyl-4-[(methyloxy)carbonyl]-2-biphenylcarboxylic acid (4.00 g, 10.8 mmol) and EDCI (4.14 g, 21.6 mmol) in dichloromethane (50 mL) was added DMAP (130 mg) and 2-aminothiazole (1.19 g, 11.9 mmol) then stirred at room temperature for 2 days. Dichloromethane (50 mL) was added and the mixture washed with water (2×20 mL), brine (10 mL) then the organics dried (MgSO₄), filtered and evaporated under reduced pressure. Purification by flash chromatography (10:1→4:1→1:1, hexane:EtOAc) gave the title compound as a pale brown solid (5.2 g, Quant, contains some solvent and 2-aminothiazole by ¹H NMR).

### 26k) 5′-[(cyclopropylamino)carbonyl]-3′-fluoro-2′-methyl-2-[(1,3-thiazol-2-ylamino)carbonyl]-4-biphenylcarboxylic acid

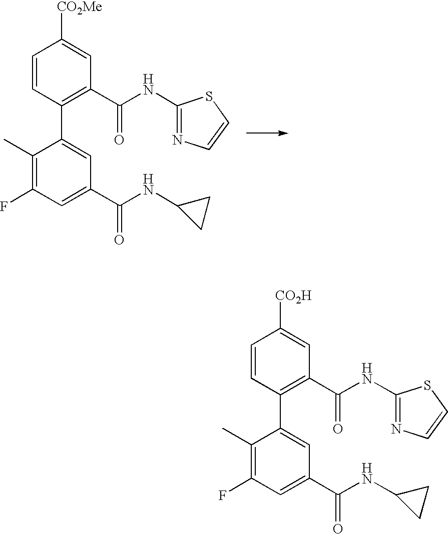

To a solution of methyl 5′-[(cyclopropylamino)carbonyl]-3′-fluoro-2′-methyl-2-[(1,3-thiazol-2-ylamino)carbonyl]-4-biphenylcarboxylate (1.00 g, 2.27 mmol) in THF (10 mL) was added water (5 mL) and lithium hydroxide. H₂O (500 mg). This was stirred at room temperature for 18 hours. The THF was removed under reduced pressure then water (10 mL) added and the mixture extracted with EtOAc (20 mL). The aqueous was acidified to pH 1 with 1M HCl_{(aq)}. The resultant precipitate was filtered off, washed with water then dried in air to give the title compound as an off-white solid (0.71 g, 74%); mpt: 280-284° C. (dec.).

### 26l) N³′-cyclopropyl-N⁴,N⁴-diethyl-5′-fluoro-6′-methyl-N²-1,3-thiazol-2-yl-2,3′,4-biphenyltricarboxamide

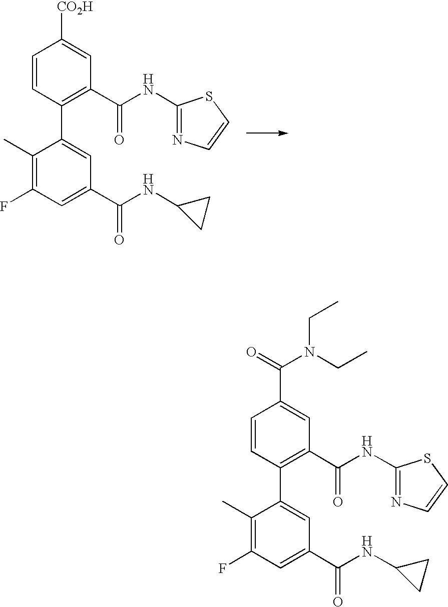

Diethylamine (17 mg, 0.226 mmol) was added in one portion to a stirred solution of 5′-[(cyclopropylamino)carbonyl]-3′-fluoro-2′-methyl-2-[(1,3-thiazol-2-ylamino)carbonyl]-4-biphenylcarboxylic acid (90 mg, 0.205 mmol), 1-(3-dimethylaminopropyl)-3-ethylcarbodiimide hydrochloride (80 mg, 0.41 mmol), and 4-dimethylaminopyridine (catalytic amount) in anhydrous dichloromethane (1 mL) under an atmosphere of argon, and the resultant reaction mixture was stirred at room temperature for 16 h before being quenched with water (5 mL). The mixture was extracted with EtOAc (2×20 ml), the organic layers separated, dried and concentrated. The crude product was purified by flash chromatography (silica, 4:1 to 1:1 hexane:EtOAc to ethyl acetate) affording the title compound (40 mg, 40%) as a white solid. LC-MS m/z 495 (M+H)⁺.

### Example 27

### N³′-cyclopropyl-5′-fluoro-6′-methyl-N²-1,3-thiazol-2-yl-N⁴-[(1S)-1,2,2-trimethylpropyl]-2,3′,4-biphenyltricarboxamide

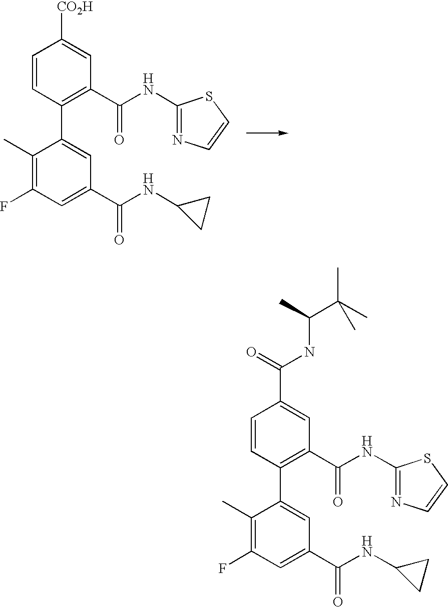

(S)-3,3-Dimethyl-2-butylamine (25 mg) was added in one portion to a stirred solution of 5′-[(cyclopropylamino)carbonyl]-3′-fluoro-2′-methyl-2-[(1,3-thiazol-2-ylamino)carbonyl]-4-biphenylcarboxylic acid (100 mg, 0.21 mmol), 1-(3-dimethylaminopropyl)-3-ethylcarbodiimide hydrochloride (87 mg, 0.455 mmol), and 4-dimethylaminopyridine (10 mg) in anhydrous dichloromethane (3 mL) under an atmosphere of argon, and the resultant reaction mixture was stirred at room temperature for 18 h. DCM (10 ml) was added and the mixture washed with water (2×5 ml), 0.5M NaOH (5 ml), brine (5 ml) then dried. Purification by flash chromatography (silica, 4:1 to 1:1 to 1:2 hexanes:EtOAc) afforded the title compound as a white solid (32 mg). LC-MS m/z 523 (M+H)⁺.

### Example 28

### N³′-cyclopropyl-5′-fluoro-6′-methyl-N²,N⁴-di-1,3-thiazol-2-yl-2,3′,4-biphenyltricarboxamide

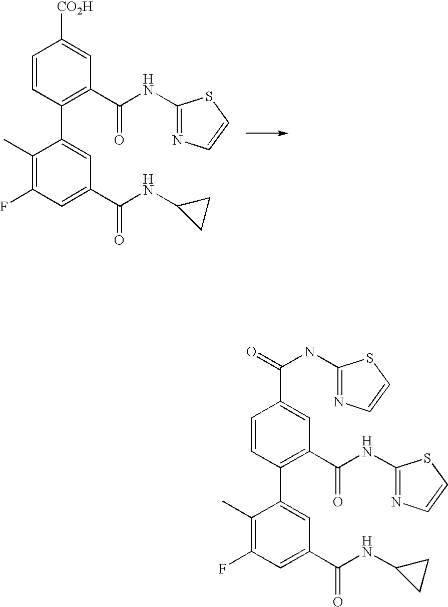

2-Aminothiazole (25 mg, 0.25 mmol) was added in one portion to a stirred solution of 5′-[(cyclopropylamino)carbonyl]-3′-fluoro-2′-methyl-2-[(1,3-thiazol-2-ylamino)carbonyl]-4-biphenylcarboxylic acid (100 mg, 0.21 mmol), 1-(3-dimethylaminopropyl)-3-ethylcarbodiimide hydrochloride (87 mg, 0.455 mmol), and 4-dimethylaminopyridine (10 mg) in anhydrous dichloromethane (3 mL) under an atmosphere of argon, and the resultant reaction mixture was stirred at room temperature for 16 h. After quenching with water (5 ml), DCM (10 ml) was added, the mixture washed with water (2×5 ml), brine (5 ml) then dried. Purification by flash chromatography (silica, 1:1 hexane:EtOAc to ethyl acetate) afforded the title compound as a white solid (20 mg). LC-MS m/z 522 (M+H)⁺.

### Example 29

### N³′-cyclopropyl-5′-fluoro-6′-methyl-4-[(4-methyl-1-piperazinyl)carbonyl]-N²-1,3-thiazol-2-yl-2,3′-biphenyldicarboxamide

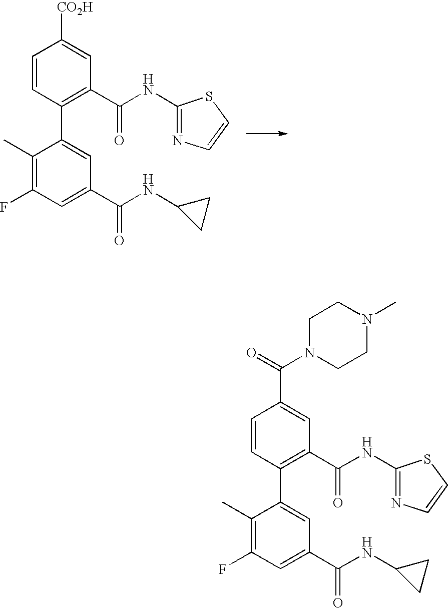

4-Methylpiperazine (25 mg, 0.25 mmol) was added in one portion to a stirred solution of 5′-[(cyclopropylamino)carbonyl]-3′-fluoro-2′-methyl-2-[(1,3-thiazol-2-ylamino)carbonyl]-4-biphenylcarboxylic acid (100 mg, 0.21 mmol), 1-(3-dimethylaminopropyl)-3-ethylcarbodiimide hydrochloride (87 mg, 0.455 mmol), and 4-dimethylaminopyridine (10 mg) in anhydrous dichloromethane (3 mL) under an atmosphere of argon, and the resultant reaction mixture was stirred at room temperature for 16 h. Further aliquot of amine (100 mg) was added and the mixture stirred at room temperature for 16 h before being quenched with water (5 mL). DCM (10 ml) added, mixture washed with water (2×5 ml), brine (5 ml) then dried. Crude product dissolved in MeOH and passed through a PEAX column eluting with MeOH. After evaporation of the solvent, the residue was purified by flash chromatography (silica, 1:1 hexane:EtOAc to ethyl acetate to 9:1 MeOH:DCM) affording the product as a white solid (60 mg). LC-MS m/z 522 (M+H)⁺.

### Example 30

### N³′-cyclopropyl-5′-fluoro-6′-methyl-N⁴-phenyl-N²-1,3-thiazol-2-yl-2,3′,4-biphenyltricarboxamide

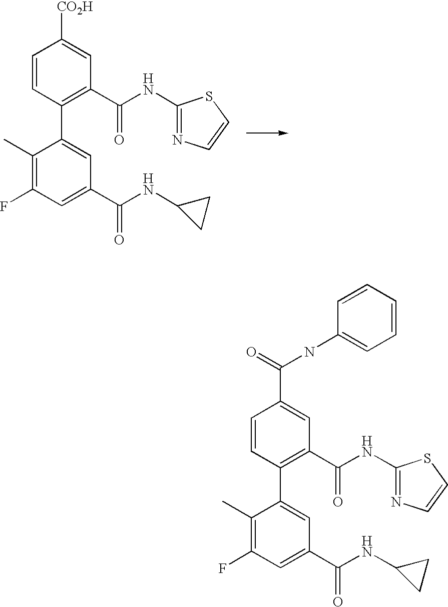

Aniline (50 mg) was added in one portion to a stirred solution of 5′-[(cyclopropylamino)carbonyl]-3′-fluoro-2′-methyl-2-[(1,3-thiazol-2-ylamino)carbonyl]-4-biphenylcarboxylic acid (100 mg, 0.21 mmol), 1-(3-dimethylaminopropyl)-3-ethylcarbodiimide hydrochloride (87 mg, 0.455 mmol), and 4-dimethylaminopyridine (10 mg) in anhydrous dichloromethane (3 mL) under an atmosphere of argon, and the resultant reaction mixture was stirred at room temperature for 18 hours. After quenching with water (5 ml), DCM (10 ml) was added, the mixture washed with water (2×5 ml), brine (5 ml) then dried. Purification by flash chromatography (silica, 4:1 to 1:1 to 1:2 hexanes:EtOAc) afforded the title compound as a white solid (25 mg). LC-MS m/z 515 (M+H)⁺.

### Example 31

### N⁴-cyclohexyl-N³′-cyclopropyl-5′-fluoro-6′-methyl-N²-1,3-thiazol-2-yl-2,3′,4-biphenyltricarboxamide

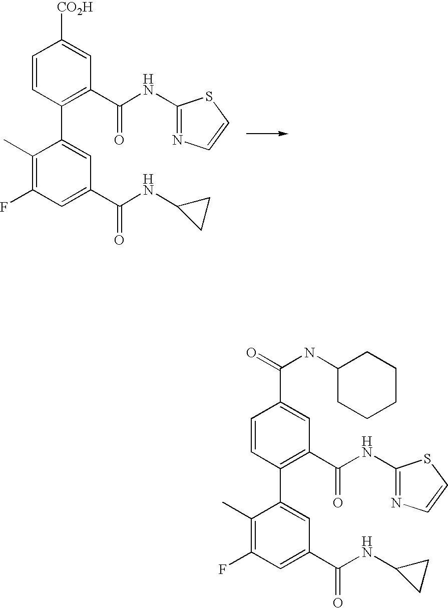

Cyclohexylamine (0.2 ml) and diisopropylethylamine (0.1 ml) were added to a stirred suspension of 5′-[(cyclopropylamino)carbonyl]-3′-fluoro-2′-methyl-2-[(1,3-thiazol-2-ylamino)carbonyl]-4-biphenylcarboxylic acid (100 mg, 0.21 mmol), O-(6-chlorobenzotriazol-1-yl)-N,N,N′,N′-tetramethyluronium hexafluorophosphate) (108 mg, 0.26 mmol) in anhydrous dichloromethane (3 mL) under an atmosphere of argon, and the resultant reaction mixture was stirred at room temperature for 3 days. After quenching with water (5 ml), DCM (15 ml) was added, the mixture washed with water (2×5 ml), brine (5 ml) then dried. Purification by flash chromatography (silica, 4:1 to 1:1 hexane:EtOAc to ethyl acetate) afforded the title compound as a white solid (20 mg). LC-MS m/z 521 (M+H)⁺.

### Example 32

### N³′-cyclopropyl-5′-fluoro-6′-methyl-N²-1,3-thiazol-2-yl-N⁴-[(1R)-1,2,2-trimethylpropyl]-2,3′,4-biphenyltricarboxamide

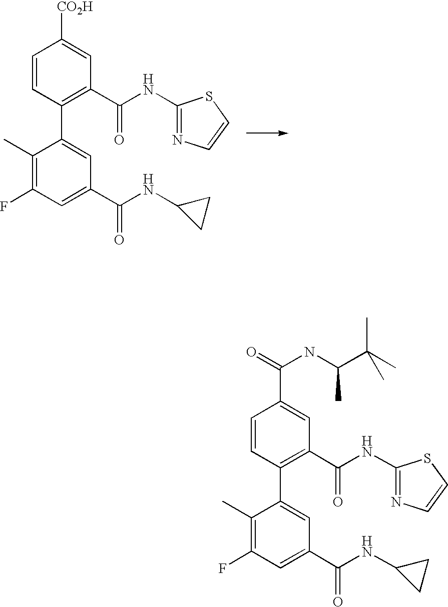

(R)-3,3-Dimethyl-2-butylamine (150 mg) was added to a stirred suspension of 5′-[(cyclopropylamino)carbonyl]-3′-fluoro-2′-methyl-2-[(1,3-thiazol-2-ylamino)carbonyl]-4-biphenylcarboxylic acid (100 mg, 0.21 mmol), triethylamine (0.1 ml), O-(6-chlorobenzotriazol-1-yl)-N,N,N′,N′-tetramethyluronium hexafluorophosphate) (116 mg, 0.26 mmol) in anhydrous dichloromethane (3 mL) under an atmosphere of argon, and the resultant reaction mixture was stirred at room temperature for 6 days. EtOAc (15 ml) was added and the mixture washed with water (2×5 ml), brine (5 ml) then dried Purification by flash chromatography (silica, 4:1 to 1:1 hexane:EtOAc to ethyl acetate) afforded the title compound as a white solid (50 mg). LC-MS m/z 523 (M+H)⁺.

### Example 33

### N³′-cyclopropyl-5′-fluoro-6′-methyl-N⁴-1H-pyrazol-5-yl-N²-1,3-thiazol-2-yl-2,3′,4-biphenyltricarboxamide

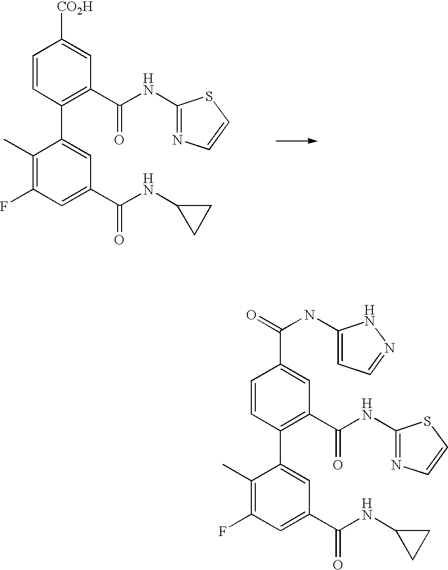

(1H)-5-Aminopyrazole (25 mg) was added to a stirred suspension of 5′-[(cyclopropylamino)carbonyl]-3′-fluoro-2′-methyl-2-[(1,3-thiazol-2-ylamino)carbonyl]-4-biphenylcarboxylic acid (100 mg, 0.21 mmol), triethylamine (0.1 ml), O-(6-chlorobenzotriazol-1-yl)-N,N,N′,N′-tetramethyluronium hexafluorophosphate) (116 mg, 0.26 mmol) in anhydrous dichloromethane (3 mL) under an atmosphere of argon, and the resultant reaction mixture was stirred at room temperature for 6 days. EtOAc (15 ml) was added and the mixture washed with water (2×5 ml), brine (5 ml) then dried. Purification by flash chromatography (silica, 1:1 hexane:EtOAc to ethyl acetate to 9:1 MeOH:DCM) afforded the title compound as a white solid (20 mg). LC-MS m/z 505 (M+H)⁺.

### Example 34

### N³′-cyclopropyl-N⁴-(3,5-dimethyl-1H-pyrazol-4-yl)-5′-fluoro-6′-methyl-N²-1,3-thiazol-2-yl-2,3′,4-biphenyltricarboxamide

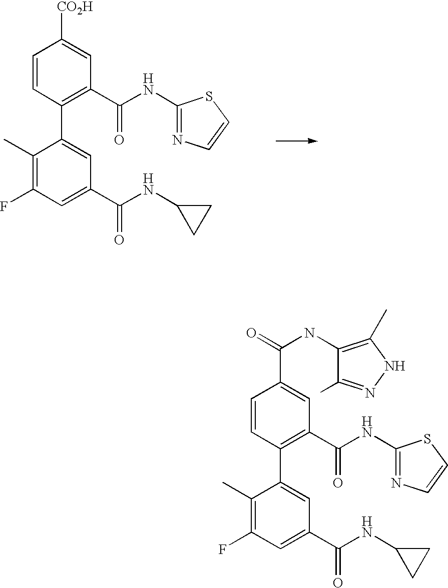

4-Amino-3,5-dimethyl-1H-pyrazole (30 mg) was added in one portion to a stirred solution of 5′-[(cyclopropylamino)carbonyl]-3′-fluoro-2′-methyl-2-[(1,3-thiazol-2-ylamino)carbonyl]-4-biphenylcarboxylic acid (100 mg, 0.21 mmol), 1-(3-dimethylaminopropyl)-3-ethylcarbodiimide hydrochloride (87 mg, 0.455 mmol), and 4-dimethylaminopyridine (10 mg) in anhydrous dichloromethane (3 mL) under an atmosphere of argon, and the resultant reaction mixture was stirred at room temperature for two weeks. EtOAc (15 ml) was added and the mixture washed with water (2×5 ml), brine (5 ml) then dried. Purification by flash chromatography (Sill cartridge, 4:1 hexanes:EtOAc to EtOAc) afforded the title compound as a white solid (30 mg). LC-MS m/z 533 (M+H)⁺.

### Example 35

### N³′-cyclopropyl-5′-fluoro-6′-methyl-N⁴-1,2,4-thiadiazol-5-yl-N²-1,3-thiazol-2-yl-2,3′,4-biphenyltricarboxamide

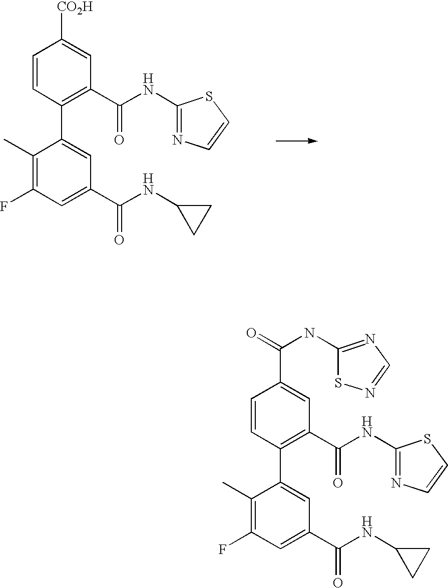

5-Amino-1,2,4-thiadiazole (25 mg) was added in one portion to a stirred solution of 5′-[(cyclopropylamino)carbonyl]-3′-fluoro-2′-methyl-2-[(1,3-thiazol-2-ylamino)carbonyl]-4-biphenylcarboxylic acid (100 mg, 0.21 mmol), 1-(3-dimethylaminopropyl)-3-ethylcarbodiimide hydrochloride (87 mg, 0.455 mmol), and 4-dimethylaminopyridine (10 mg) in anhydrous dichloromethane (3 mL) under an atmosphere of argon, and the resultant reaction mixture was stirred at room temperature for two weeks. EtOAc (15 ml) was added and the mixture washed with water (2×5 ml), brine (5 ml) then dried. Purification by flash chromatography (Sill cartridge, 4:1 hexanes:EtOAc to EtOAc) afforded the title compound as a white solid (30 mg). LC-MS m/z 523 (M+H)⁺.

### Example 36

### N³′-cyclopropyl-N⁴-[(1R)-1,2-dimethylpropyl]-5′-fluoro-6′-methyl-N²-1,3-thiazol-2-yl-2,3′,4-biphenyltricarboxamide

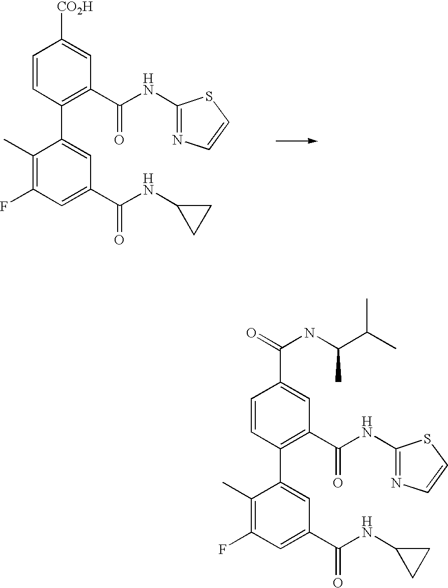

(1R)-1,2-Dimethylpropylamine (60 mg) was added in one portion to a stirred solution of 5′-[(cyclopropylamino)carbonyl]-3′-fluoro-2′-methyl-2-[(1,3-thiazol-2-ylamino)carbonyl]-4-biphenylcarboxylic acid (100 mg, 0.21 mmol), 1-(3-dimethylaminopropyl)-3-ethylcarbodiimide hydrochloride (90 mg, 0.455 mmol), and 4-dimethylaminopyridine (10 mg) in anhydrous dichloromethane (3 mL) under an atmosphere of argon, and the resultant reaction mixture was stirred at room temperature for five days. EtOAc (15 ml) was added and the mixture washed with water (2×5 ml), brine (5 ml) then dried. Purification by flash chromatography (silica, 9:1 to 4:1 to 1:2 hexanes:EtOAc) afforded the product as a white solid (50 mg). LC-MS m/z 509 (M+H)⁺.

### Example 37

### N³′-cyclopropyl-N⁴-[(1S)-1,2-dimethylpropyl]-5′-fluoro-6′-methyl-N²-1,3-thiazol-2-yl-2,3′,4-biphenyltricarboxamide

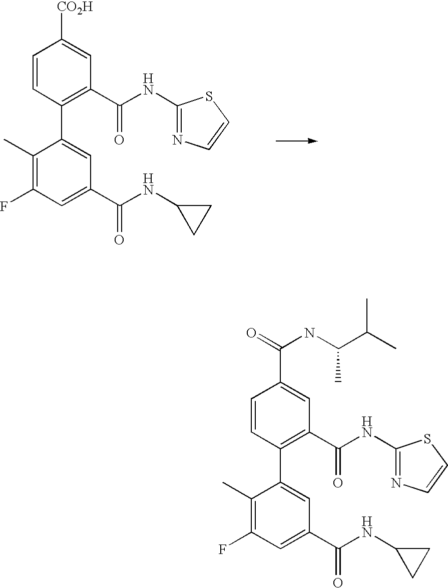

(1S)-1,2-Dimethylpropylamine (60 mg) was added in one portion to a stirred solution of 5′-[(cyclopropylamino)carbonyl]-3′-fluoro-2′-methyl-2-[(1,3-thiazol-2-ylamino)carbonyl]-4-biphenylcarboxylic acid (100 mg, 0.21 mmol), 1-(3-dimethylaminopropyl)-3-ethylcarbodiimide hydrochloride (90 mg, 0.455 mmol), and 4-dimethylaminopyridine (10 mg) in anhydrous dichloromethane (3 mL) under an atmosphere of argon, and the resultant reaction mixture was stirred at room temperature for five days. EtOAc (15 ml) was added and the mixture washed with water (2×5 ml), brine (5 ml) then dried. Purification by flash chromatography (silica, 9:1 to 4:1 to 1:2 hexanes:EtOAc) afforded the title compound as a white solid (50 mg). LC-MS m/z 509 (M+H)⁺.

### Example 38

### N³′-cyclopropyl-N⁴-(cyclopropylmethyl)-5′-fluoro-6′-methyl-N²-1,3-thiazol-2-yl-2,3′, 4-biphenyltricarboxamide

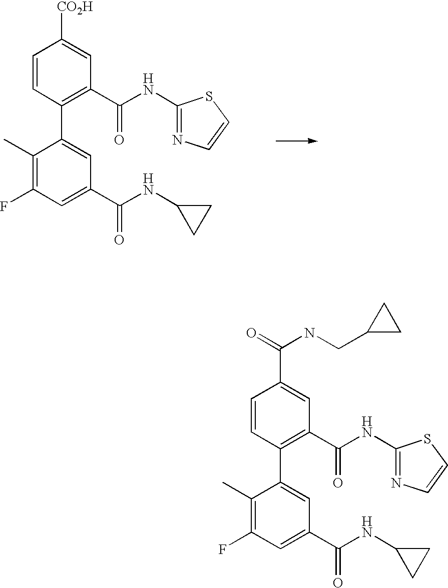

Cyclopropanemethylamine (50 mg) was added in one portion to a stirred solution of 5′-[(cyclopropylamino)carbonyl]-3′-fluoro-2′-methyl-2-[(1,3-thiazol-2-ylamino)carbonyl]-4-biphenylcarboxylic acid (100 mg, 0.21 mmol), 1-(3-dimethylaminopropyl)-3-ethylcarbodiimide hydrochloride (90 mg, 0.455 mmol), and 4-dimethylaminopyridine (10 mg) in anhydrous dichloromethane (3 mL) under an atmosphere of argon, and the resultant reaction mixture was stirred at room temperature for five days. EtOAc (15 ml) was added and the mixture washed with water (2×5 ml), brine (5 ml) then dried. Purification by flash chromatography (silica, 9:1 to 4:1 to 1:2 hexanes:EtOAc) afforded the title compound as a white solid (45 mg). LC-MS m/z 493 (M+H)⁺.

### Example 39

### N³′-cyclopropyl-5′-fluoro-6′-methyl-N²-1,3-thiazol-2-yl-N⁴-(2,2,2-trifluoroethyl)-2,3′,4-biphenyltricarboxamide

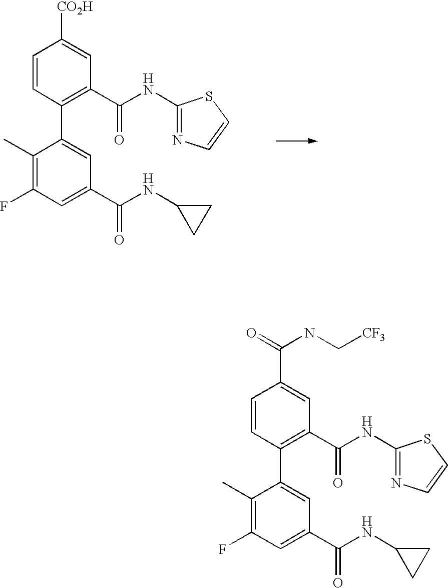

2,2,2-Trifluoroethylamine (70 mg) was added in one portion to a stirred solution of 5′-[(cyclopropylamino)carbonyl]-3′-fluoro-2′-methyl-2-[(1,3-thiazol-2-ylamino)carbonyl]-4-biphenylcarboxylic acid (100 mg, 0.21 mmol), 1-(3-dimethylaminopropyl)-3-ethylcarbodiimide hydrochloride (90 mg, 0.455 mmol), and 4-dimethylaminopyridine (10 mg) in anhydrous dichloromethane (3 mL) under an atmosphere of argon, and the resultant reaction mixture was stirred at room temperature for five days. EtOAc (15 ml) was added and the mixture washed with water (2×5 ml), brine (5 ml) then dried. Purification by flash chromatography (silica, 9:1 to 4:1 to 1:2 hexanes:EtOAc) afforded the title compound as a white solid (60 mg). LC-MS m/z 521 (M+H)⁺.

### Example 40

### N³′-cyclopropyl-5′-fluoro-6′-methyl-N⁴-[(1R)-1-phenylethyl]-N²-1,3-thiazol-2-yl-2,3′,4-biphenyltricarboxamide

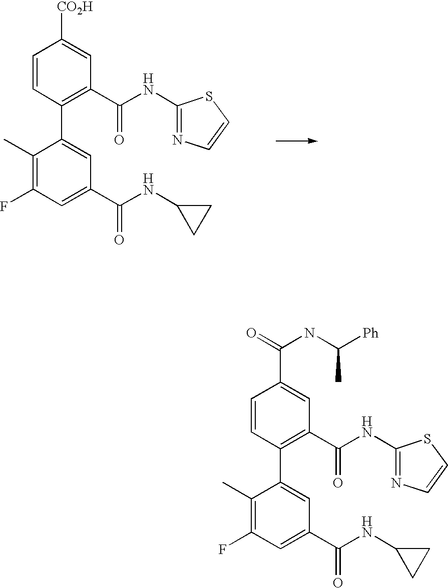

(1R)-(+)-1-Phenylethylamine (85 mg) was added in one portion to a stirred solution of 5′-[(cyclopropylamino)carbonyl]-3′-fluoro-2′-methyl-2-[(1,3-thiazol-2-ylamino)carbonyl]-4-biphenylcarboxylic acid (100 mg, 0.21 mmol), 1-(3-dimethylaminopropyl)-3-ethylcarbodiimide hydrochloride (90 mg, 0.455 mmol), and 4-dimethylaminopyridine (10 mg) in anhydrous dichloromethane (3 mL) under an atmosphere of argon, and the resultant reaction mixture was stirred at room temperature for five days. EtOAc (15 ml) was added and the mixture washed with water (2×5 ml), brine (5 ml) then dried. Purification by flash chromatography (silica, 9:1 to 4:1 to 1:2 hexanes:EtOAc) afforded the title compound as a white solid (60 mg). LC-MS m/z 543 (M+H)⁺.

### Example 41

### N³′-cyclopropyl-5′-fluoro-6′-methyl-N⁴-(phenylmethyl)-N²-1,3-thiazol-2-yl-2,3′,4-biphenyltricarboxamide)

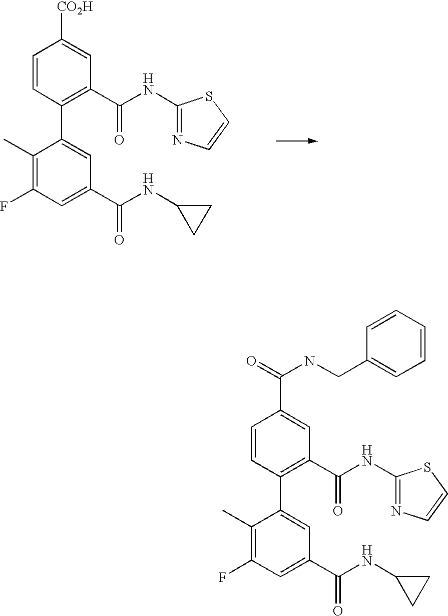

Benzylamine (70 mg) was added in one portion to a stirred solution of 5′-[(cyclopropylamino)carbonyl]-3′-fluoro-2′-methyl-2-[(1,3-thiazol-2-ylamino)carbonyl]-4-biphenylcarboxylic acid (100 mg, 0.21 mmol), 1-(3-dimethylaminopropyl)-3-ethylcarbodiimide hydrochloride (90 mg, 0.455 mmol), and 4-dimethylaminopyridine (110 mg) in anhydrous dichloromethane (3 mL) under an atmosphere of argon, and the resultant reaction mixture was stirred at room temperature for two weeks. EtOAc (15 ml) was added and the mixture washed with water (2×5 ml), brine (5 ml) then dried. Purification by flash chromatography (Si II cartridge 4:1 to 1:2 hexanes:EtOAc to EtOAc) afforded the title compound as a white solid (40 mg). LC-MS m/z 529 (M+H)⁺.

### Example 42

### N⁴-(cyclohexylmethyl)-N³′-cyclopropyl-5′-fluoro-6′-methyl-N²-1,3-thiazol-2-yl-2,3′,4-biphenyltricarboxamide

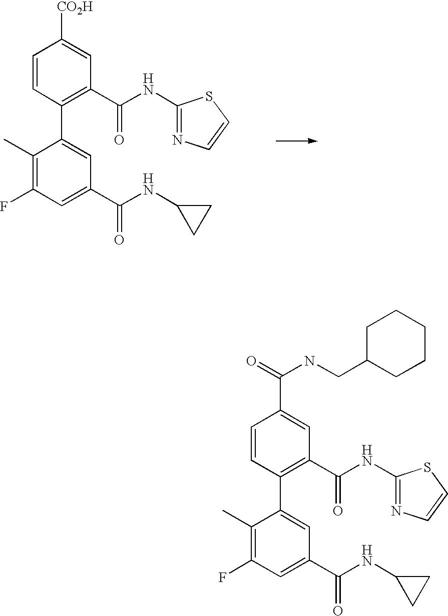

Cyclohexanemethylamine (60 mg) was added in one portion to a stirred solution of 5′-[(cyclopropylamino)carbonyl]-3′-fluoro-2′-methyl-2-[(1,3-thiazol-2-ylamino)carbonyl]-4-biphenylcarboxylic acid (100 mg, 0.21 mmol), 1-(3-dimethylaminopropyl)-3-ethylcarbodiimide hydrochloride (90 mg, 0.455 mmol), and 4-dimethylaminopyridine (10 mg) in anhydrous dichloromethane (3 mL) under an atmosphere of argon, and the resultant reaction mixture was stirred at room temperature for five days. EtOAc (15 ml) was added and the mixture washed with water (2×5 ml), brine (5 ml) then dried. Purification by flash chromatography (Si II cartridge 4:1 to 1:2 hexanes:EtOAc to EtOAc) afforded the title compound as a white solid (20 mg). LC-MS m/z 535 (M+H)⁺.

### Example 43

### N³′-cyclopropyl-5′-fluoro-6′-methyl-N⁴-(2-methylpropyl)-N²-1,3-thiazol-2-yl-2,3′,4-biphenyltricarboxamide

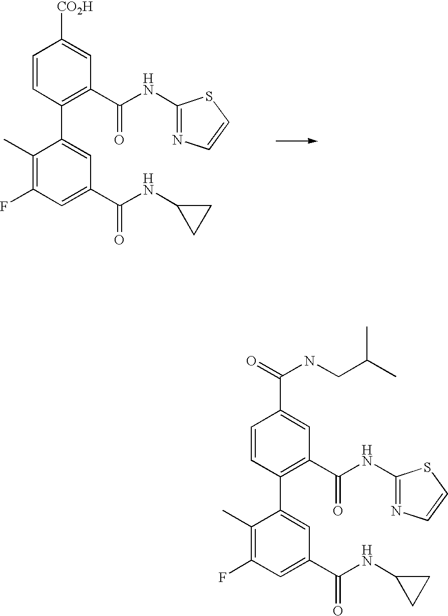

Isobutylamine (60 mg) was added in one portion to a stirred solution of 5′-[(cyclopropylamino)carbonyl]-3′-fluoro-2′-methyl-2-[(1,3-thiazol-2-ylamino)carbonyl]-4-biphenylcarboxylic acid (100 mg, 0.21 mmol), 1-(3-dimethylaminopropyl)-3-ethylcarbodiimide hydrochloride (90 mg, 0.455 mmol), and 4-dimethylaminopyridine (10 mg) in anhydrous dichloromethane (3 mL) under an atmosphere of argon, and the resultant reaction mixture was stirred at room temperature for five days. EtOAc (15 ml) was added and the mixture washed with water (2×5 ml), brine (5 ml) then dried. Purification by flash chromatography (Si II cartridge 4:1 to 1:2 hexanes:EtOAc to EtOAc) afforded the title compound as a white solid (20 mg). LC-MS m/z 495 (M+H)⁺.

### Example 44

### N³′-cyclopropyl-N-[3-(dibutylamino)propyl]-5′-fluoro-6′-methyl-N²-1,3-thiazol-2-yl-2,3′,4-biphenyltricarboxamide

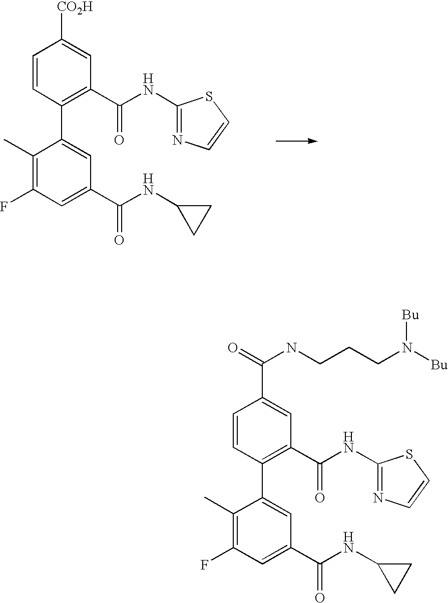

3-(Dibutylamino)-1-propylamine (60 mg) was added in one portion to a stirred solution of 5′-[(cyclopropylamino)carbonyl]-3′-fluoro-2′-methyl-2-[(1,3-thiazol-2-ylamino)carbonyl]-4-biphenylcarboxylic acid (100 mg, 0.228 mmol), 1-(3-dimethylaminopropyl)-3-ethylcarbodiimide hydrochloride (90 mg, 0.46 mmol), and 4-dimethylaminopyridine (10 mg) in anhydrous dichloromethane (3 mL) under an atmosphere of argon, and the resultant reaction mixture was stirred at room temperature for five days. EtOAc (15 ml) was added and the mixture washed with water (2×5 ml), brine (5 ml) then dried. Purification by flash chromatography (Si II cartridge 4:1 to 1:2 hexanes:EtOAc to EtOAc) afforded the title compound as a white solid (50 mg). LC-MS m/z 608 (M+H)⁺.

### Example 45

### N³′-cyclopropyl-N⁴-[3-(dimethylamino)-2,2-dimethylpropyl]-5′-fluoro-6′-methyl-N²-1,3-thiazol-2-yl-2,3′,4-biphenyltricarboxamide

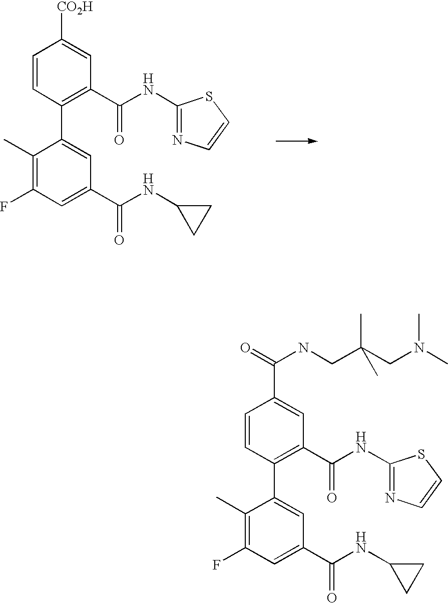

N,N-2,2-Tetramethyl-1,3-propanediamine (60 mg) was added in one portion to a stirred solution of 5′-[(cyclopropylamino)carbonyl]-3′-fluoro-2′-methyl-2-[(1,3-thiazol-2-ylamino)carbonyl]-4-biphenylcarboxylic acid (100 mg, 0.228 mmol), 1-(3-dimethylaminopropyl)-3-ethylcarbodiimide hydrochloride (90 mg, 0.46 mmol), and 4-dimethylaminopyridine (10 mg) in anhydrous dichloromethane (3 mL) under an atmosphere of argon, and the resultant reaction mixture was stirred at room temperature for two weeks. EtOAc (15 ml) was added and the mixture washed with water (2×5 ml), brine (5 ml) then dried. Purification by flash chromatography (Si II cartridge 4:1 to 1:2 hexanes:EtOAc to EtOAc) afforded the title compound as a white solid (35 mg). LC-MS m/z 552 (M+H)⁺.

### Example 46

### N³′-cyclopropyl-5′-fluoro-6′-methyl-N⁴-[(1R)-1-phenylethyl]-N²-1,3-thiazol-2-yl-2,3′,4-biphenyltricarboxamide

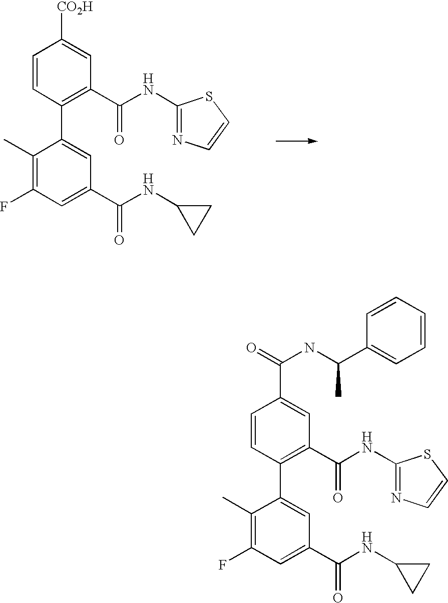

(S)-(−)-1-Phenylethylamine (70 mg) was added in one portion to a stirred solution of 5′-[(cyclopropylamino)carbonyl]-3′-fluoro-2′-methyl-2-[(1,3-thiazol-2-ylamino)carbonyl]-4-biphenylcarboxylic acid (100 mg, 0.21 mmol), 1-(3-dimethylaminopropyl)-3-ethylcarbodiimide hydrochloride (90 mg, 0.455 mmol), and 4-dimethylaminopyridine (10 mg) in anhydrous dichloromethane (3 mL) under an atmosphere of argon, and the resultant reaction mixture was stirred at room temperature for five days. EtOAc (15 ml) was added and the mixture washed with water (2×5 ml), brine (5 ml) then dried. Purification by flash chromatography (Si II cartridge 4:1 to 1:2 hexanes:EtOAc to EtOAc) afforded the title compound as a white solid (60 mg). LC-MS m/z 543 (M+H)⁺.

### Example 47

### N³′-cyclopropyl-5′-fluoro-6′-methyl-N⁴-(3-methylbutyl)-N²-1,3-thiazol-2-yl-2,3′,4-biphenyltricarboxamide

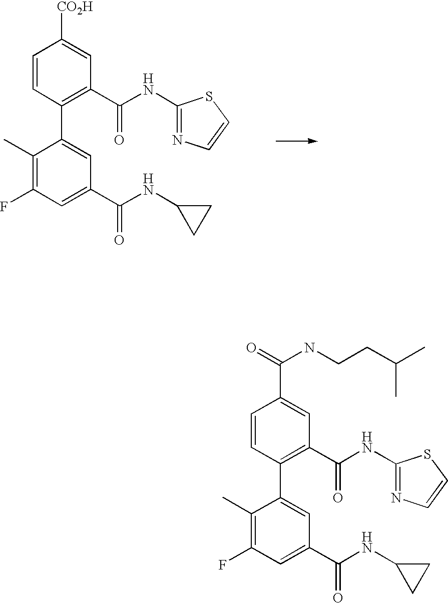

Isoamylamine (60 mg) was added in one portion to a stirred solution of 5′-[(cyclopropylamino)carbonyl]-3′-fluoro-2′-methyl-2-[(1,3-thiazol-2-ylamino)carbonyl]-4-biphenylcarboxylic acid (100 mg, 0.21 mmol), 1-(3-dimethylaminopropyl)-3-ethylcarbodiimide hydrochloride (90 mg, 0.455 mmol), and 4-dimethylaminopyridine (10 mg) in anhydrous dichloromethane (3 mL) under an atmosphere of argon, and the resultant reaction mixture was stirred at room temperature for five days. EtOAc (15 ml) was added and the mixture washed with water (2×5 ml), brine (5 ml) then dried. Purification by flash chromatography (Si II cartridge 4:1 to 1:2 hexanes:EtOAc to EtOAc) afforded the title compound as a white solid (13 mg). LC-MS m/z 509 (M+H)⁺.

### Example 48

### N³′-cyclopropyl-N⁴-(1-ethylpropyl)-5′-fluoro-6′-methyl-N²-1,3-thiazol-2-yl-2,3′,4-biphenyltricarboxamide

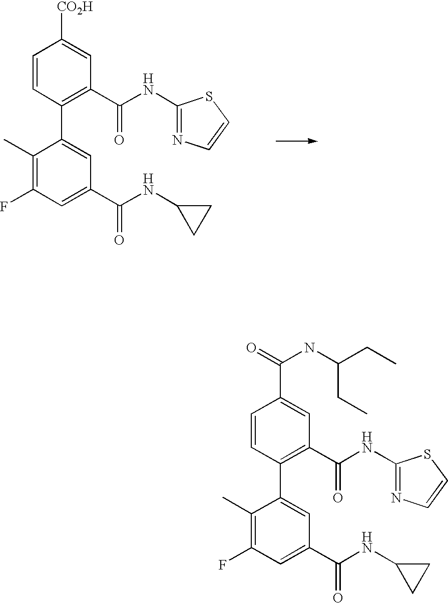

3-Pentylamine (60 mg) was added in one portion to a stirred solution of 5′-[(cyclopropylamino)carbonyl]-3′-fluoro-2′-methyl-2-[(1,3-thiazol-2-ylamino)carbonyl]-4-biphenylcarboxylic acid (100 mg, 0.21 mmol), 1-(3-dimethylaminopropyl)-3-ethylcarbodiimide hydrochloride (90 mg, 0.455 mmol), and 4-dimethylaminopyridine (10 mg) in anhydrous dichloromethane (3 mL) under an atmosphere of argon, and the resultant reaction mixture was stirred at room temperature for five days. EtOAc (15 ml) was added and the mixture washed with water (2×5 ml), brine (5 ml) then dried. Purification by flash chromatography (Si II cartridge 4:1 to 1:2 hexanes:EtOAc to EtOAc) afforded the title compound as a white solid (14 mg). LC-MS m/z 509 (M+H)⁺.

### Example 49

### N³′-cyclopropyl-N⁴-[(3,4-dimethylphenyl)methyl]-5′-fluoro-6′-methyl-N²-1,3-thiazol-2-yl-2,3′,4-biphenyltricarboxamide

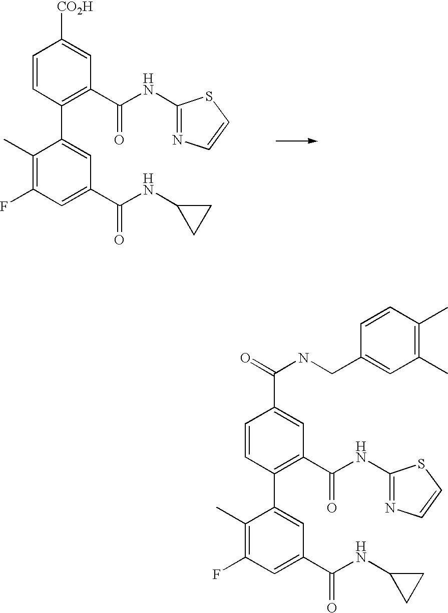

3,4-Dimethylbenzylamine (60 mg) was added in one portion to a stirred solution of 5′-[(cyclopropylamino)carbonyl]-3′-fluoro-2′-methyl-2-[(1,3-thiazol-2-ylamino)carbonyl]-4-biphenylcarboxylic acid (100 mg, 0.21 mmol), 1-(3-dimethylaminopropyl)-3-ethylcarbodiimide hydrochloride (90 mg, 0.455 mmol), and 4-dimethylaminopyridine (10 mg) in anhydrous dichloromethane (3 mL) under an atmosphere of argon, and the resultant reaction mixture was stirred at room temperature for five days. EtOAc (15 ml) was added and the mixture washed with water (2×5 ml), brine (5 ml) then dried. Purification by flash chromatography (silica, 9:1 to 4:1 to 1:2 hexanes:EtOAc) afforded the title compound as a white solid (60 mg).). LC-MS m/z 557 (M+H)⁺.

### Example 50

### N³′-cyclopropyl-5′-fluoro-N⁴,6′-dimethyl-N²-1,3-thiazol-2-yl-2,3′,4-biphenyltricarboxamide

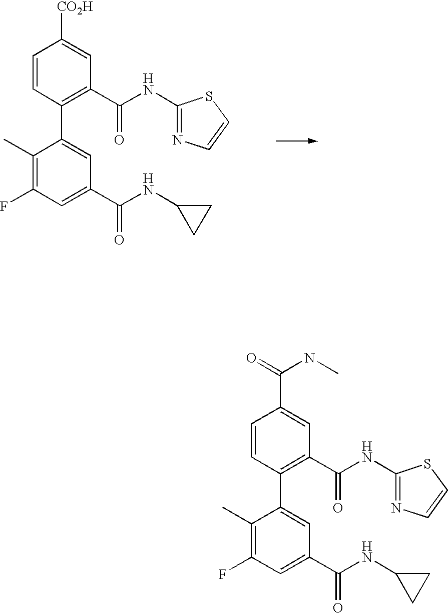

Methylamine hydrochloride (60 mg) was added in one portion to a stirred solution of 5′-[(cyclopropylamino)carbonyl]-3′-fluoro-2′-methyl-2-[(1,3-thiazol-2-ylamino)carbonyl]-4-biphenylcarboxylic acid (100 mg, 0.228 mmol), 1-(3-dimethylaminopropyl)-3-ethylcarbodiimide hydrochloride (90 mg, 0.46 mmol), triethylamine (0.2 ml) and 4-dimethylaminopyridine (10 mg) in anhydrous dichloromethane (3 mL) under an atmosphere of argon, and the resultant reaction mixture was stirred at room temperature for five days. EtOAc (15 ml) was added and the mixture washed with water (2×5 ml), brine (5 ml) then dried. Purification by flash chromatography (silica, 9:1 to 4:1 to 1:2 hexanes:EtOAc) afforded the title compound as an off-white solid (12 mg). LC-MS m/z 453 (M+H)⁺.

### Example 51

### N³′-cyclopropyl-5′-fluoro-6′-methyl-N⁴-[2-(1-pyrrolidinyl)ethyl]-N²-1,3-thiazol-2-yl-2,3′,4-biphenyltricarboxamide

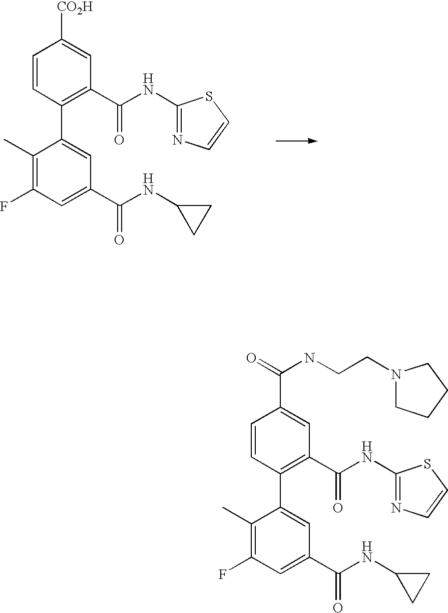

N-(2-Aminoethyl)pyrrolidine (60 mg) was added in one portion to a stirred solution of 5′-[(cyclopropylamino)carbonyl]-3′-fluoro-2′-methyl-2-[(1,3-thiazol-2-ylamino)carbonyl]-4-biphenylcarboxylic acid (100 mg, 0.21 mmol), 1-(3-dimethylaminopropyl)-3-ethylcarbodiimide hydrochloride (90 mg, 0.455 mmol), and 4-dimethylaminopyridine (10 mg) in anhydrous dichloromethane (3 mL) under an atmosphere of argon, and the resultant reaction mixture was stirred at room temperature for two weeks. EtOAc (15 ml) was added and the mixture washed with water (2×5 ml), brine (5 ml) then dried. Purification by flash chromatography (Si II cartridge 4:1 to 1:2 hexanes:EtOAc to EtOAc) afforded the title compound as a white solid (12 mg). LC-MS m/z 536 (M+H)⁺.

### Example 52

### N³′-cyclopropyl-N⁴-(2,2-dimethylpropyl)-5′-fluoro-6′-methyl-2,3′,4-biphenyltricarboxamide

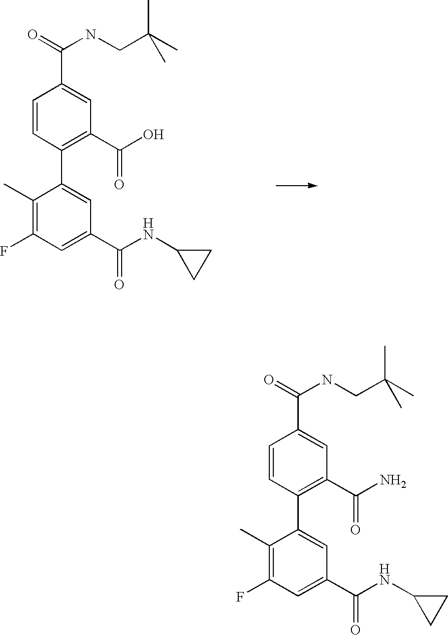

To a solution of 5′-[(Cyclopropylamino)carbonyl]-4-{[(2,2-dimethylpropyl)-amino]carbonyl}-3′-fluoro-2′-methyl-2-biphenylcarboxylic acid (27.8 mg, 0.0653 mmol) (example 1c) in DMF (0.65 mL) was added HBTU (24.8 mg, 0.0653 mmol), Et₃N (18.4 uL, 0.131 mmol) and ammonia (2.0M in isopropanol, 0.0655 mL, 0.131 mmol). The solution was stirred at room temperature over night. The reaction mixture was quenched with H₂O (1 drop), concentrated and filtered. Purification via HPLC (Gilson) then afforded the title compound (9 mg, 32%). LC-MSm/z 426 (M+H)⁺.

### Example 53

### N²-cyclopentyl-N³′-cyclopropyl-N⁴-(2,2-dimethylpropyl)-5′-fluoro-6′-methyl-2,3′,4-biphenyltricarboxamide

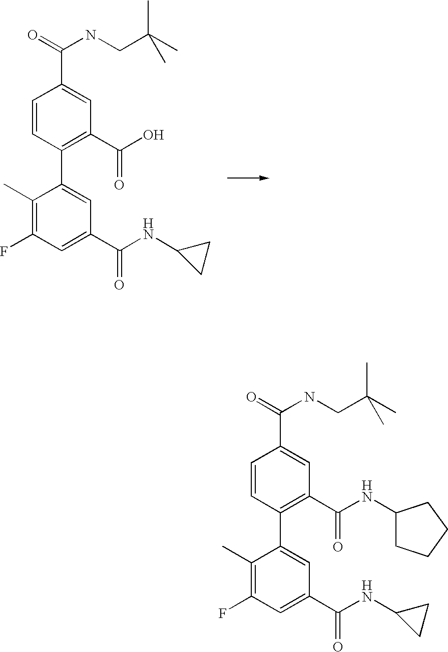

To a solution of 5′-[(Cyclopropylamino)carbonyl]-4-{[(2,2-dimethylpropyl)-amino]carbonyl}-3′-fluoro-2′-methyl-2-biphenylcarboxylic acid (21.3 mg, 0.050 mmol) in CH₂Cl₂ (2.0 mL) was added HBTU (20.9 mg, 0.055 mmol), Et₃N (28.1 uL, 0.20 mmol) and cyclopentanamine (5.5 uL, 0.055 mmol). The solution was stirred at room temperature over night. The reaction mixture was quenched with H₂O (1 drop), DMSO (0.5 mL), concentrated and filtered. Purification via HPLC (Gilson) then afforded the title compound (13 mg, 53%). LC-MS m/z 494 (M+H)⁺.

### Example 54

### N²-(cyclohexylmethyl)-N³′-cyclopropyl-N⁴-(2,2-dimethylpropyl)-5′-fluoro-6′-methyl-2,3′,4-biphenyltricarboxamide

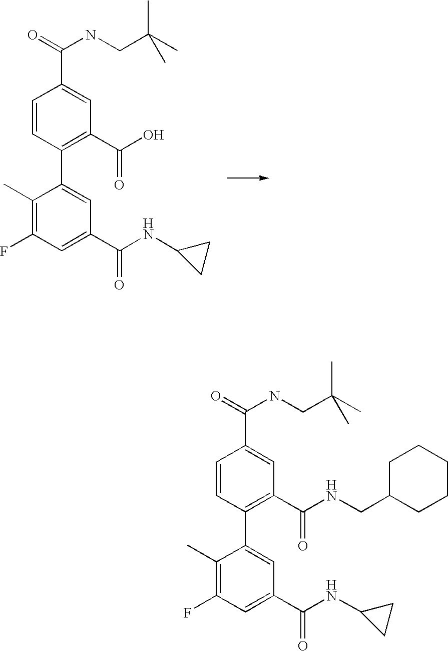

To a solution of 5′-[(Cyclopropylamino)carbonyl]-4-{[(2,2-dimethylpropyl)-amino]carbonyl}-3′-fluoro-2′-methyl-2-biphenylcarboxylic acid (21.3 mg, 0.050 mmol) in CH₂Cl₂ (2.0 mL) was added HBTU (20.9 mg, 0.055 mmol), Et₃N (28.1 uL, 0.20 mmol) and (cyclohexylmethyl)amine (7.2 uL, 0.055 mmol). The solution was stirred at room temperature over night. The reaction mixture was quenched with H₂O (1 drop), DMSO (0.5 mL), concentrated and filtered. Purification via HPLC (Gilson) then afforded the title compound (15 mg, 58%). LC-MS m/z 522 (M+H)⁺.

### Example 55

### N³′-cyclopropyl-N⁴-(2,2-dimethylpropyl)-5′-fluoro-6′-methyl-N²-phenyl-2,3′,4-biphenyltricarboxamide

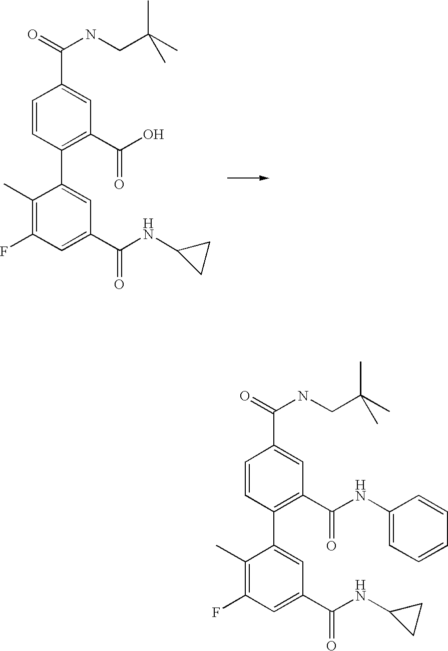

To a solution of 5′-[(Cyclopropylamino)carbonyl]-4-{[(2,2-dimethylpropyl)-amino]carbonyl}-3′-fluoro-2′-methyl-2-biphenylcarboxylic acid (21.3 mg, 0.050 mmol) in CH₂Cl₂ (2.0 mL) was added HBTU (20.9 mg, 0.055 mmol), Et₃N (28.1 uL, 0.20 mmol) and aniline (5.0 uL, 0.055 mmol). The solution was stirred at room temperature over night. The reaction mixture was quenched with H₂O (1 drop), DMSO (0.5 mL), concentrated and filtered. Purification via HPLC (Gilson) then afforded the title compound (13 mg, 53%). LC-MS m/z 502 (M+H)⁺.

### Example 56

### N³′-cyclopropyl-N²-(3,4-difluorophenyl)-N⁴-(2,2-dimethylpropyl)-5′-fluoro-6′-methyl-2,3′,4-biphenyltricarboxamide

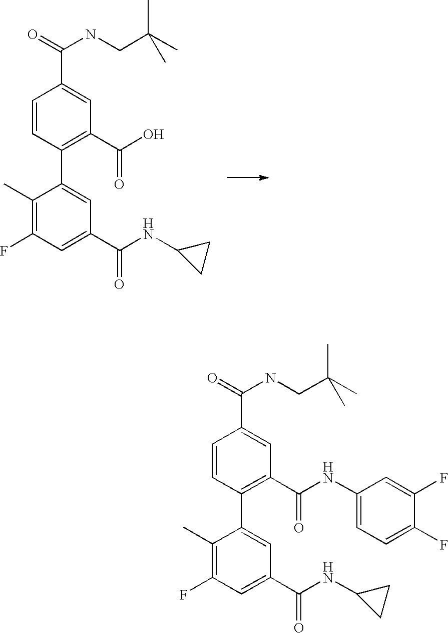

To a solution of 5′-[(Cyclopropylamino)carbonyl]-4-{[(2,2-dimethylpropyl)-amino]carbonyl}-3′-fluoro-2′-methyl-2-biphenylcarboxylic acid (21.3 mg, 0.050 mmol) in CH₂Cl₂ (2.0 mL) was added HBTU (20.9 mg, 0.055 mmol), Et₃N (28.1 uL, 0.20 mmol) and 3,4-difloroaniline (5.4 uL, 0.055 mmol). The solution was stirred at room temperature over night. The reaction mixture was quenched with H₂O (1 drop), DMSO (0.5 mL), concentrated and filtered. Purification via HPLC (Gilson) then afforded the title compound (9 mg, 34%). LC-MS m/z 538 (M+H)⁺.

### Example 57

### N³′-cyclopropyl-N²-(4-fluorophenyl)-N⁴-(2,2-dimethylpropyl)-5′-fluoro-6′-methyl-2,3′,4-biphenyltricarboxamide

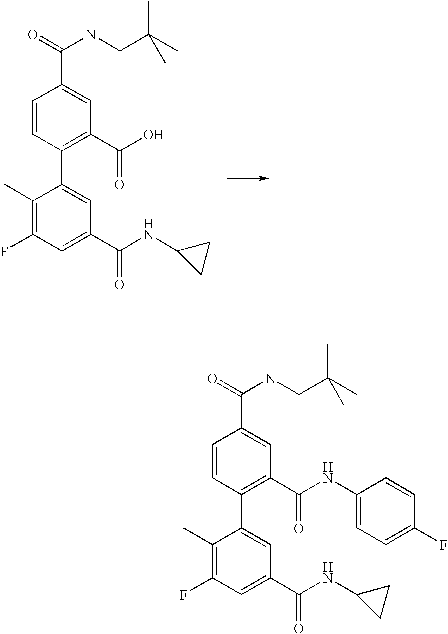

To a solution of 5′-[(Cyclopropylamino)carbonyl]-4-{[(2,2-dimethylpropyl)-amino]carbonyl}-3′-fluoro-2′-methyl-2-biphenylcarboxylic acid (21.3 mg, 0.050 mmol) in CH₂Cl₂ (2.0 mL) was added HBTU (20.9 mg, 0.055 mmol), Et₃N (28.1 uL, 0.20 mmol) and 4-floroaniline (5.2 uL, 0.055 mmol). The solution was stirred at room temperature over night. The reaction mixture was quenched with H₂O (1 drop), DMSO (0.5 mL), concentrated and filtered. Purification via HPLC (Gilson) then afforded the title compound (12 mg, 47%). LC-MS m/z 520 (M+H)⁺.

### Example 58

### N³′-cyclopropyl-N⁴-(2,2-dimethylpropyl)-5′-fluoro-6′-methyl-N²-(2,2,2-trifluoroethyl)-2,3′,4-biphenyltricarboxamide

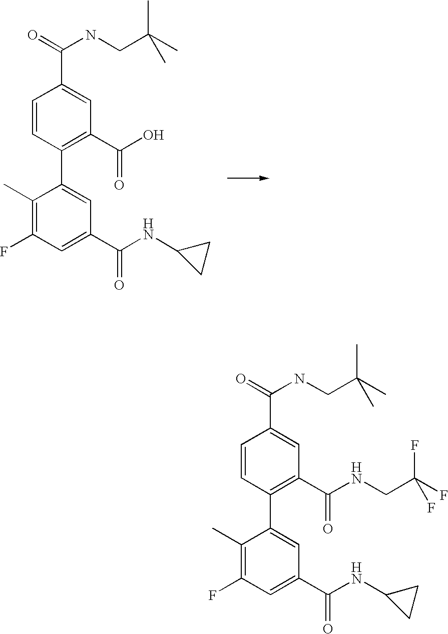

To a solution of 5′-[(Cyclopropylamino)carbonyl]-4-{[(2,2-dimethylpropyl)-amino]carbonyl}-3′-fluoro-2′-methyl-2-biphenylcarboxylic acid (21.3 mg, 0.050 mmol) in CH₂Cl₂ (2.0 mL) was added HBTU (20.9 mg, 0.055 mmol), Et₃N (28.1 uL, 0.20 mmol) and (2,2,2-trifluoroethyl)amine (7.5 mg, 0.055 mmol). The solution was stirred at room temperature over night. The reaction mixture was quenched with H₂O (1 drop), DMSO (0.5 mL), concentrated and filtered. Purification via HPLC (Gilson) then afforded the title compound (12 mg, 48%). LC-MS m/z 508 (M+H)⁺.

### Example 59

### N³′-cyclopropyl-N⁴-(2,2-dimethylpropyl)-N²-[3-(ethyloxy)propyl]-5′-fluoro-6′-methyl-2,3′, 4-biphenyltricarboxamide

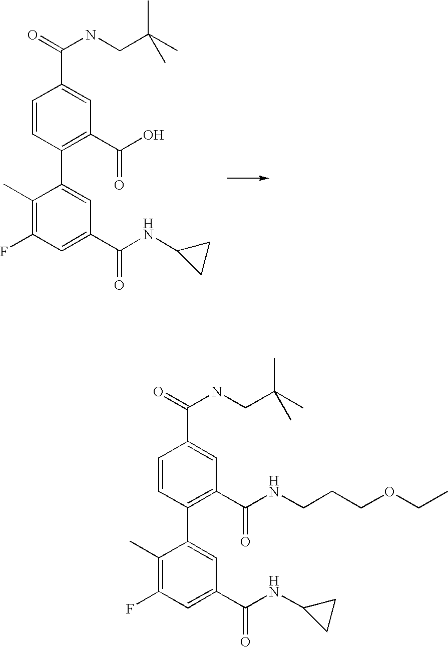

To a solution of 5′-[(Cyclopropylamino)carbonyl]-4-{[(2,2-dimethylpropyl)-amino]carbonyl}-3′-fluoro-2′-methyl-2-biphenylcarboxylic acid (42.6 mg, 0.10 mmol) in DMF (2.0 mL) was added HBTU (41.7 mg, 0.11 mmol), Et₃N (56.2 uL, 0.40 mmol) and [3-(ethyloxy)propyl]amine (18.0 uL, 0.15 mmol). The solution was stirred at room temperature over night. The reaction mixture was quenched with H₂O (1 drop), DMSO (0.5 mL), concentrated and filtered. Purification via HPLC (Gilson) then afforded the title compound (34 mg, 66%). LC-MS m/z 512 (M+H)⁺.

### Example 60

### N³′-cyclopropyl-N⁴-(2,2-dimethylpropyl)-5′-fluoro-N²-(5-hydroxypentyl)-6′-methyl-2,3′,4-biphenyltricarboxamide

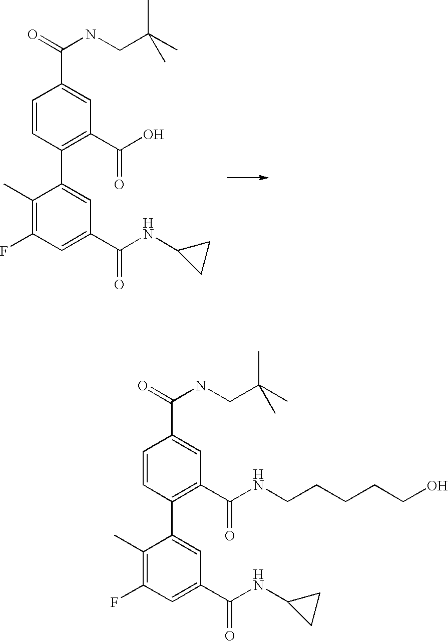

To a solution of 5′-[(Cyclopropylamino)carbonyl]-4-{[(2,2-dimethylpropyl)-amino]carbonyl}-3′-fluoro-2′-methyl-2-biphenylcarboxylic acid (42.6 mg, 0.10 mmol) in DMF (2.0 mL) was added HBTU (41.7 mg, 0.11 mmol), Et₃N (56.2 uL, 0.40 mmol) and 5-amino-1-pentanol (15.5 uL, 0.15 mmol). The solution was stirred at room temperature over night. The reaction mixture was quenched with H₂O (1 drop), DMSO (0.5 mL), concentrated and filtered. Purification via HPLC (Gilson) then afforded the title compound (32 mg, 62%). LC-MS m/z 512 (M+H)⁺.

### Example 61

### N³′-cyclopropyl-N⁴-(2,2-dimethylpropyl)-5′-fluoro-N²-[3-(1H-imidazol-1-yl)propyl]-6′-methyl-2,3′,4-biphenyltricarboxamide

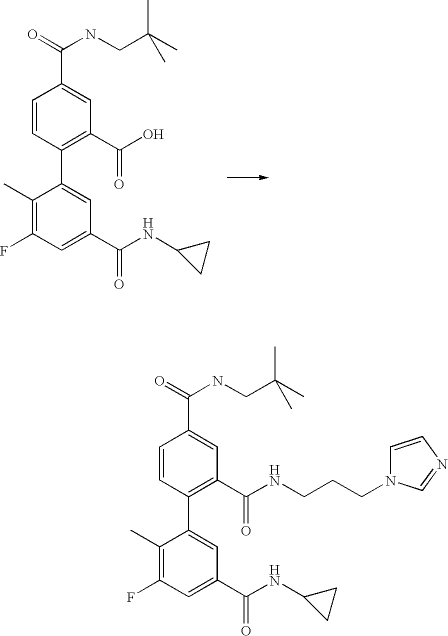

To a solution of 5′-[(Cyclopropylamino)carbonyl]-4-{[(2,2-dimethylpropyl)-amino]carbonyl}-3′-fluoro-2′-methyl-2-biphenylcarboxylic acid (42.6 mg, 0.10 mmol) in DMF (2.0 mL) was added HBTU (41.7 mg, 0.11 mmol), Et₃N (56.2 uL, 0.40 mmol) and [3-(1H-imidazol-1-yl)propyl]amine (17.9 uL, 0.15 mmol). The solution was stirred at room temperature over night. The reaction mixture was quenched with H₂O (1 drop), DMSO (0.5 mL), concentrated and filtered. Purification via HPLC (Gilson) then afforded the title compound (36 mg, 67%). LC-MS m/z 534 (M+H)⁺.

### Example 62

### N³′-cyclopropyl-N⁴-(2,2-dimethylpropyl)-5′-fluoro-6′-methyl-N²-[3-(4-methyl-1-piperazinyl)propyl]-2,3′,4-biphenyltricarboxamide

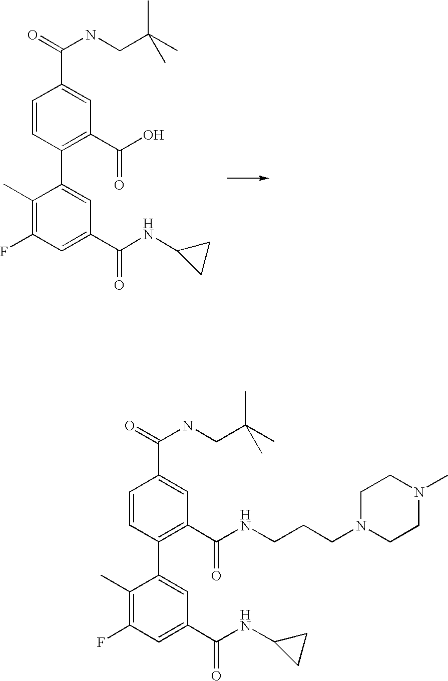

To a solution of 5′-[(Cyclopropylamino)carbonyl]-4-{[(2,2-dimethylpropyl)-amino]carbonyl}-3′-fluoro-2′-methyl-2-biphenylcarboxylic acid (42.6 mg, 0.10 mmol) in DMF (2.0 mL) was added HBTU (41.7 mg, 0.11 mmol), Et₃N (56.2 uL, 0.40 mmol) and [3-(4-methyl-1-piperazinyl)propyl]amine (27.8 uL, 0.15 mmol). The solution was stirred at room temperature over night. The reaction mixture was quenched with H₂O (1 drop), DMSO (0.5 mL), concentrated and filtered.

Purification via HPLC (Gilson) then afforded the title compound (37 mg, 65%). LC-MS m/z 566 (M+H)⁺.

### Example 63

### N²-{2-[4-(aminosulfonyl)phenyl]ethyl}-N³′-cyclopropyl-N⁴-(2,2-dimethylpropyl)-5′-fluoro-6′-methyl-2,3′,4-biphenyltricarboxamide

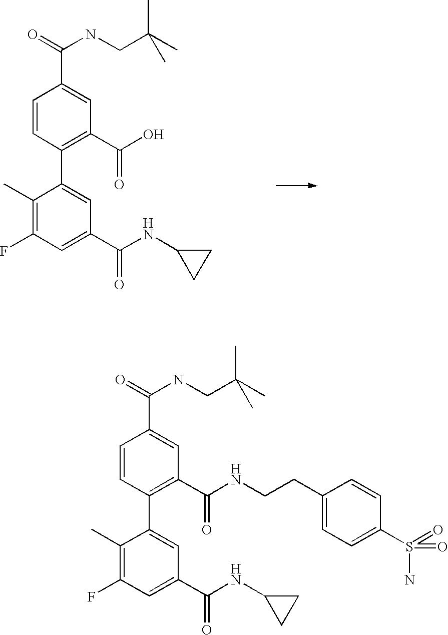

To a solution of 5′-[(Cyclopropylamino)carbonyl]-4-{[(2,2-dimethylpropyl)-amino]carbonyl}-3′-fluoro-2′-methyl-2-biphenylcarboxylic acid (42.6 mg, 0.10 mmol) in DMF (2.0 mL) was added HBTU (41.7 mg, 0.11 mmol), Et₃N (56.2 uL, 0.40 mmol) and 4-(2-aminoethyl)benzenesulfonamide (30.0 mg, 0.15 mmol). The solution was stirred at room temperature over night. The reaction mixture was quenched with H₂O (1 drop), DMSO (0.5 mL), concentrated and filtered. Purification via HPLC (Gilson) then afforded the title compound (35 mg, 57%). LC-MS m/z 609 (M+H)⁺.

### Example 64

### N³′-cyclopropyl-N²-[3-(dibutylamino)propyl]-N⁴-(2,2-dimethylpropyl)-5′-fluoro-6′-methyl-2,3′,4-biphenyltricarboxamide

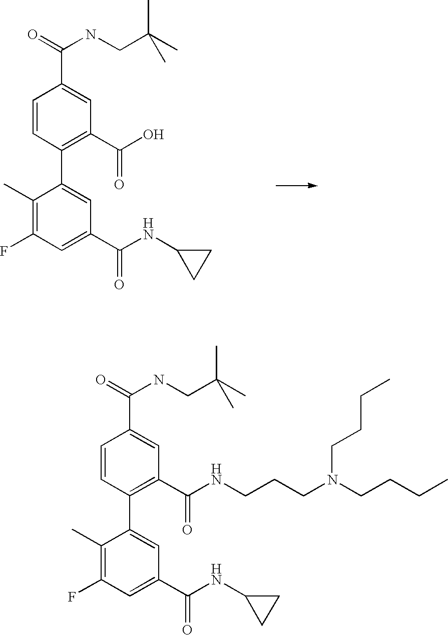

To a solution of 5′-[(Cyclopropylamino)carbonyl]-4-{[(2,2-dimethylpropyl)-amino]carbonyl}-3′-fluoro-2′-methyl-2-biphenylcarboxylic acid (42.6 mg, 0.10 mmol) in DMF (2.0 mL) was added HBTU (41.7 mg, 0.11 mmol), Et₃N (56.2 uL, 0.40 mmol) and N,N-dibutyl-1,3-propanediamine (33.8 uL, 0.15 mmol). The solution was stirred at room temperature over night. The reaction mixture was quenched with H₂O (1 drop), DMSO (0.5 mL), concentrated and filtered. Purification via HPLC (Gilson) then afforded the title compound (29 mg, 48%). LC-MS m/z 595 (M+H)⁺.

### Example 65

### N³-cyclopropyl-N⁴′-(2,2-dimethylpropyl)-5-fluoro-6-methyl-2′-{[4-(1-pyrrolidinyl)-1-piperidinyl]carbonyl}-3,4′-biphenyldicarboxamide

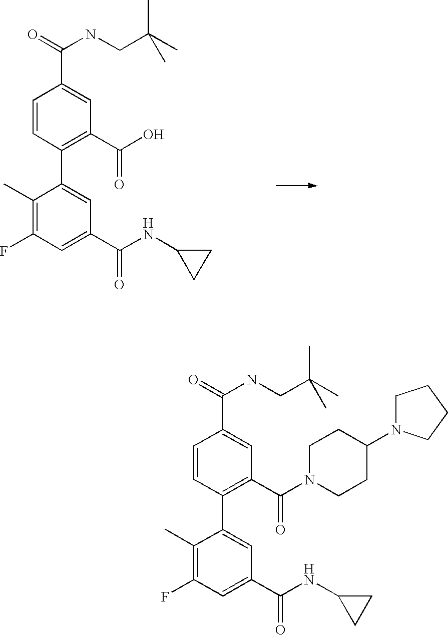

To a solution of 5′-[(Cyclopropylamino)carbonyl]-4-{[(2,2-dimethylpropyl)-amino]carbonyl}-3′-fluoro-2′-methyl-2-biphenylcarboxylic acid (42.6 mg, 0.10 mmol) in DMF (2.0 mL) was added HBTU (41.7 mg, 0.11 mmol), Et₃N (56.2 uL, 0.40 mmol) and 4-(1-pyrrolidinyl)piperidine (23.1 mg, 0.15 mmol). The solution was stirred at room temperature over night. The reaction mixture was quenched with H₂O (1 drop), DMSO (0.5 mL), concentrated and filtered. Purification via HPLC (Gilson) then afforded the title compound (25 mg, 44%). LC-MS m/z 563 (M+H)⁺.

### Example 66

### N³′-cyclopropyl-N⁴-(2,2-dimethylpropyl)-5′-fluoro-6′-methyl-N²-(4-phenylbutyl)-2,3′,4-biphenyltricarboxamide

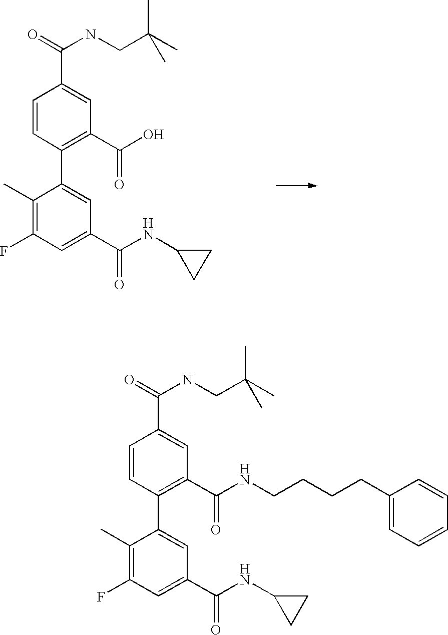

To a solution of 5′-[(Cyclopropylamino)carbonyl]-4-{[(2,2-dimethylpropyl)-amino]carbonyl}-3′-fluoro-2′-methyl-2-biphenylcarboxylic acid (42.6 mg, 0.10 mmol) in DMF (2.0 mL) was added HBTU (41.7 mg, 0.11 mmol), Et₃N (56.2 uL, 0.40 mmol) and (4-phenylbutyl)amine (23.7 uL, 0.15 mmol). The solution was stirred at room temperature over night. The reaction mixture was quenched with H₂O (1 drop), DMSO (0.5 mL), concentrated and filtered. Purification via HPLC (Gilson) then afforded the title compound (33 mg, 59%). LC-MS m/z 558 (M+H)⁺.

### Example 67

### N²-(5-cyanopentyl)-N³′-cyclopropyl-N⁴-(2,2-dimethylpropyl)-5′-fluoro-6′-methyl-2,3′,4-biphenyltricarboxamide

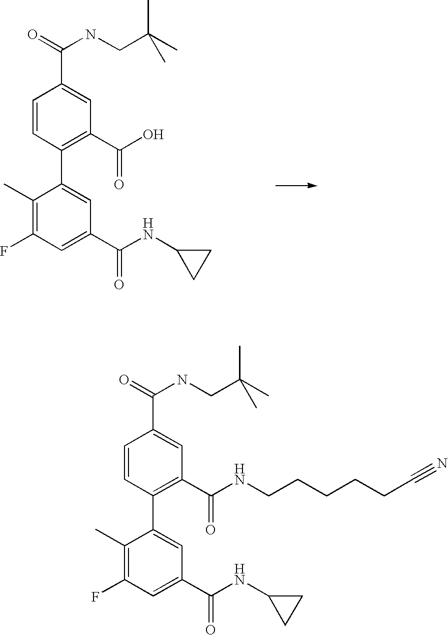

To a solution of 5′-[(Cyclopropylamino)carbonyl]-4-{[(2,2-dimethylpropyl)-amino]carbonyl}-3′-fluoro-2′-methyl-2-biphenylcarboxylic acid (42.6 mg, 0.10 mmol) in DMF (2.0 mL) was added HBTU (41.7 mg, 0.11 mmol), Et₃N (56.2 uL, 0.40 mmol) and 6-aminohexanenitrile (18.6 uL, 0.15 mmol). The solution was stirred at room temperature over night. The reaction mixture was quenched with H₂O (1 drop), DMSO (0.5 mL), concentrated and filtered. Purification via HPLC (Gilson) then afforded the title compound (34 mg, 65%). LC-MS m/z 521 (M+H)⁺.

### Example 68

### N³′-cyclopropyl-N⁴-(2,2-dimethylpropyl)-5′-fluoro-6′-methyl-N²-(2-phenylethyl)-2,3′,4-biphenyltricarboxamide

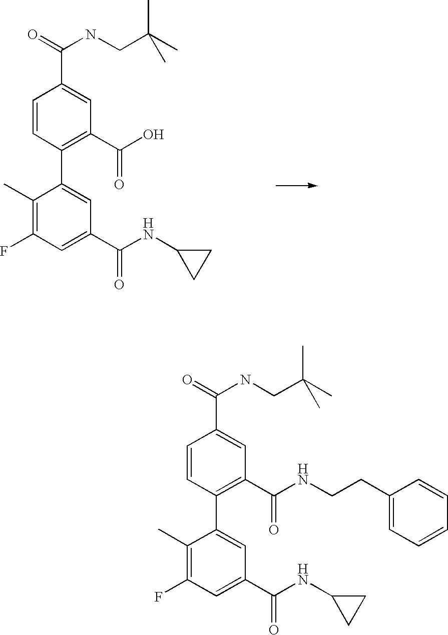

To a solution of 5′-[(Cyclopropylamino)carbonyl]-4-{[(2,2-dimethylpropyl)-amino]carbonyl}-3′-fluoro-2′-methyl-2-biphenylcarboxylic acid (42.6 mg, 0.10 mmol) in DMF (2.0 mL) was added HBTU (41.7 mg, 0.11 mmol), Et₃N (56.2 uL, 0.40 mmol) and (2-phenylethyl)amine (18.8 uL, 0.15 mmol). The solution was stirred at room temperature over night. The reaction mixture was quenched with H₂O (1 drop), DMSO (0.5 mL), concentrated and filtered. Purification via HPLC (Gilson) then afforded the title compound (32 mg, 60%). LC-MS m/z 530 (M+H)⁺.

### Example 69

### N³′-cyclopropyl-N⁴-(2,2-dimethylpropyl)-5′-fluoro-6′-methyl-N²-{3-[(1-methylethyl)oxy]propyl}-2,3′,4-biphenyltricarboxamide

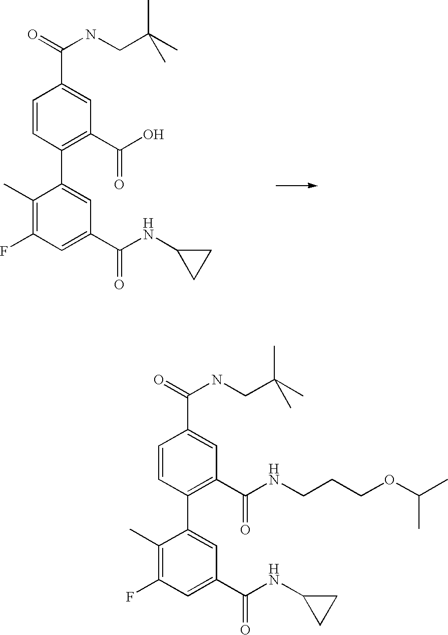

To a solution of 5′-[(Cyclopropylamino)carbonyl]-4-{[(2,2-dimethylpropyl)-amino]carbonyl}-3′-fluoro-2′-methyl-2-biphenylcarboxylic acid (42.6 mg, 0.10 mmol) in DMF (2.0 mL) was added HBTU (41.7 mg, 0.11 mmol), Et₃N (56.2 uL, 0.40 mmol) and {3-[(1-methylethyl)oxy]propyl}amine (20.7 uL, 0.15 mmol). The solution was stirred at room temperature over night. The reaction mixture was quenched with H₂O (1 drop), DMSO (0.5 mL), concentrated and filtered. Purification via HPLC (Gilson) then afforded the title compound (32 mg, 60%). LC-MS m/z 526 (M+H)⁺.

### Example 70

### N³′-cyclopropyl-N⁴-(2,2-dimethylpropyl)-5′-fluoro-6′-methyl-N²-[2-(1-pyrrolidinyl)ethyl]-2,3′,4-biphenyltricarboxamide

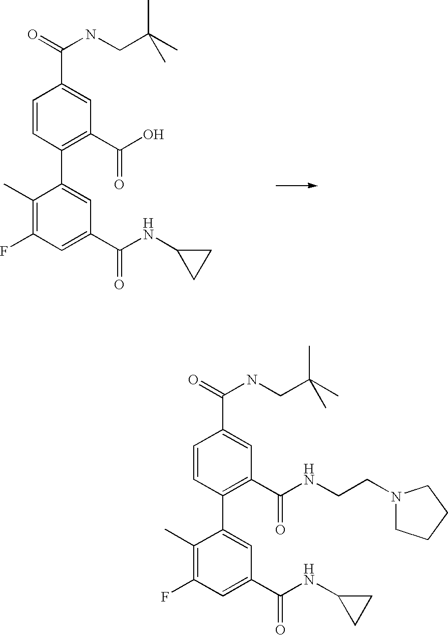

To a solution of 5′-[(Cyclopropylamino)carbonyl]-4-{[(2,2-dimethylpropyl)-amino]carbonyl}-3′-fluoro-2′-methyl-2-biphenylcarboxylic acid (42.6 mg, 0.10 mmol) in CH₂Cl₂ (1.0 mL) was added EDC (19.2 mg, 0.10 mmol), HOBT (1.4 mg, 0.01 mmol), Et₃N (28.1 uL, 0.20 mmol) and [2-(1-pyrrolidinyl)ethyl]amine (12.6 uL, 0.10 mmol). The solution was stirred at room temperature over night. The reaction mixture was quenched with H₂O (1 drop), DMSO (0.5 mL), concentrated and filtered. Purification via HPLC (Gilson) then afforded the title compound (51 mg, 80%). LC-MS m/z 523 (M+H)⁺.

### Example 71

### N³′-cyclopropyl-N⁴-(2,2-dimethylpropyl)-N²-[(1-ethyl-2-pyrrolidinyl)methyl]-5′-fluoro-6′-methyl-2,3′,4-biphenyltricarboxamide

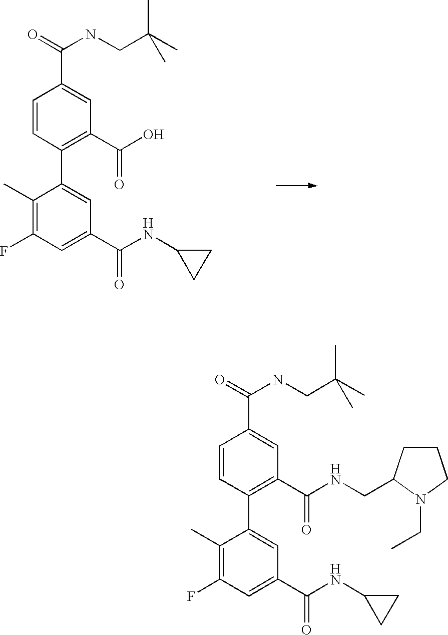

To a solution of 5′-[(Cyclopropylamino)carbonyl]-4-{[(2,2-dimethylpropyl)-amino]carbonyl}-3′-fluoro-2′-methyl-2-biphenylcarboxylic acid (42.6 mg, 0.10 mmol) in CH₂Cl₂ (1.0 mL) was added EDC (19.2 mg, 0.10 mmol), HOBT (1.4 mg, 0.01 mmol), Et₃N (28.1 uL, 0.20 mmol) and [(1-ethyl-2-pyrrolidinyl)methyl]amine (12.8 mg, 0.10 mmol). The solution was stirred at room temperature over night. The reaction mixture was quenched with H₂O (1 drop), DMSO (0.5 mL), concentrated and filtered. Purification via HPLC (Gilson) then afforded the title compound (47 mg, 88%). LC-MS m/z 537 (M+H)⁺.

### Example 72

### N⁴-(2,2-dimethylpropyl)-5′-fluoro-N³′-[(2S)-2-hydroxypropyl]-6′-methyl-2,3′,4-biphenyltricarboxamide

### 72a) 1,1-dimethylethyl 2′-(aminocarbonyl)-4′-{[(2,2-dimethylpropyl)amino]carbonyl}-5-fluoro-6-methyl-3-biphenylcarboxylate

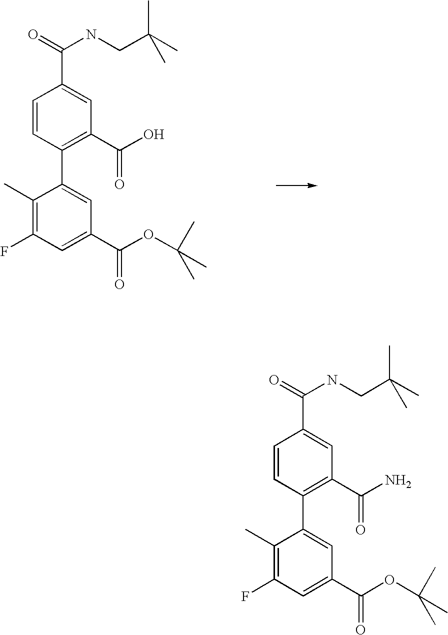

To a solution of 5′-{[(1,1-dimethylethyl)oxy]carbonyl}-4-{[(2,2-dimethylpropyl)amino]carbonyl}-3′-fluoro-2′-methyl-2-biphenylcarboxylic acid (0.82 g, 1.85 mmol) (example 10 g) in CH₂Cl₂ (18.5 mL) was added EDC (0.355 g, 1.85 mmol), HOBT (25.7 mg, 0.19 mmol), Et₃N (0.52 mL, 3.70 mmol) and ammonia (0.5M in dioxane, 7.4 mL, 3.70 mmol). The solution was stirred at room temperature over night. The reaction mixture was concentrated and filtered. Purification via combiflash then afforded the title compound (274 mg, 28%). LC-MS m/z 537 (M+H)⁺.

### 72b) 2′-(aminocarbonyl)-4′-{[(2,2-dimethylpropyl)amino]carbonyl}-5-fluoro-6-methyl-3-biphenylcarboxylic acid

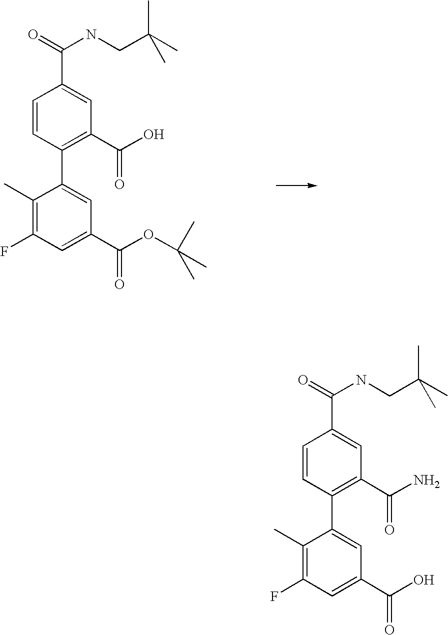

To a solution of 1,1-dimethylethyl 2′-(aminocarbonyl)-4′-{[(2,2-dimethylpropyl)amino]carbonyl}-5-fluoro-6-methyl-3-biphenylcarboxylate (22.1 mg, 0.05 mmol) in H₂O (0.5 mL) and MeOH (1.0 mL) was added potassium hydroxide (28.1 mg, 0.5 mmol). The solution was microwaved at 100° C. for 15 min. The reaction mixture was added with acetic acid to PH=4, concentrated and filtered. Purification via HPLC (Gilson) then afforded the title compound (14 mg, 73%). LC-MS m/z 387 (M+H)⁺.

### 72c) N⁴-(2,2-dimethylpropyl)-5′-fluoro-N³′-[(2S)-2-hydroxypropyl]-6′-methyl-2,3′,4-biphenyltricarboxamide

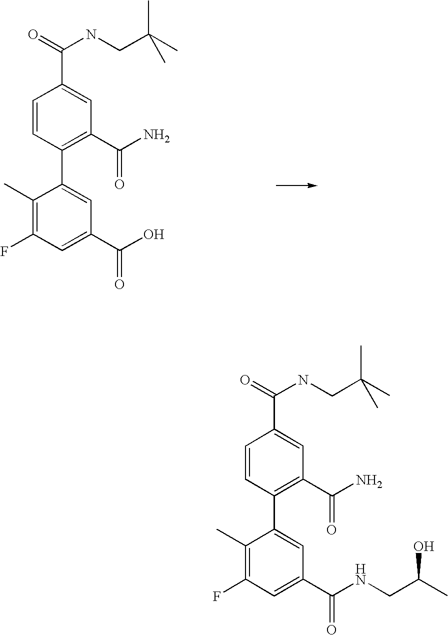

To a solution of 2′-(aminocarbonyl)-4′-{[(2,2-dimethylpropyl)amino]carbonyl}-5-fluoro-6-methyl-3-biphenylcarboxylic acid (23.2 mg, 0.06 mmol) in DMF (0.6 mL) was added HBTU (25.0 mg, 0.066 mmol), Et₃N (16.9 uL, 0.12 mmol) and (2S)-1-amino-2-propanol (9.7 uL, 0.09 mmol). The solution was stirred at room temperature over night. The reaction mixture was quenched with H₂O (1 drop), DMSO (0.5 mL), concentrated and filtered. Purification via HPLC (Gilson) then afforded the title compound (13 mg, 49%). LC-MS m/z 444 (M+H)⁺.

### Example 73

### N⁴-(2,2-dimethylpropyl)-5′-fluoro-N³′-[(2R)-2-hydroxypropyl]-6′-methyl-2,3′,4-biphenyltricarboxamide

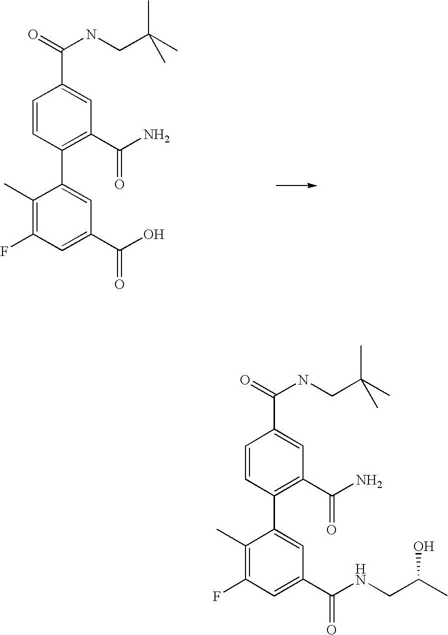

To a solution of 2′-(aminocarbonyl)-4′-{[(2,2-dimethylpropyl)amino]carbonyl}-5-fluoro-6-methyl-3-biphenylcarboxylic acid (23.2 mg, 0.06 mmol) in DMF (0.6 mL) was added HBTU (25.0 mg, 0.066 mmol), Et₃N (16.9 uL, 0.12 mmol) and (2R)-1-amino-2-propanol (9.7 uL, 0.09 mmol). The solution was stirred at room temperature over night. The reaction mixture was quenched with H₂O (1 drop), DMSO (0.5 mL), concentrated and filtered. Purification via HPLC (Gilson) then afforded the title compound (15 mg, 57%). LC-MS m/z 444 (M+H)⁺.

### Example 74

### N⁴-(2,2-dimethylpropyl)-5′-fluoro-N³′-[(1S)-2-hydroxy-1-methylethyl]-6′-methyl-2,3′,4-biphenyltricarboxamide

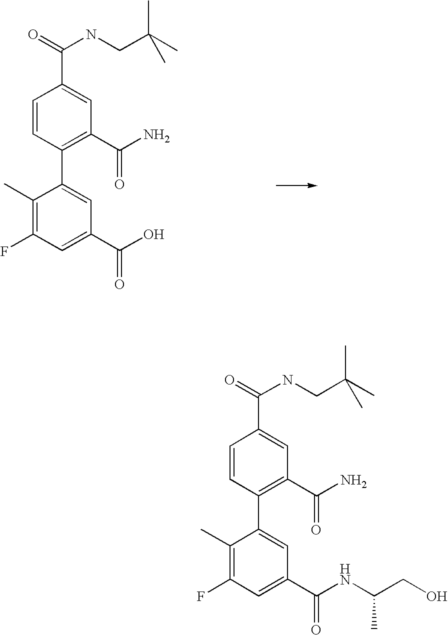

To a solution of 2′-(aminocarbonyl)-4′-{[(2,2-dimethylpropyl)amino]carbonyl}-5-fluoro-6-methyl-3-biphenylcarboxylic acid (23.2 mg, 0.06 mmol) in DMF (0.6 mL) was added HBTU (25.0 mg, 0.066 mmol), Et₃N (16.9 uL, 0.12 mmol) and (2S)-2-amino-1-propanol (9.7 uL, 0.09 mmol). The solution was stirred at room temperature over night. The reaction mixture was quenched with H₂O (1 drop), DMSO (0.5 mL), concentrated and filtered. Purification via HPLC (Gilson) then afforded the title compound (15 mg, 57%). LC-MS m/z 444 (M+H)⁺.

### Example 75

### N⁴-(2,2-dimethylpropyl)-5′-fluoro-N³′-[(1R)-2-hydroxy-1-methylethyl]-6′-methyl-2,3′,4-biphenyltricarboxamide

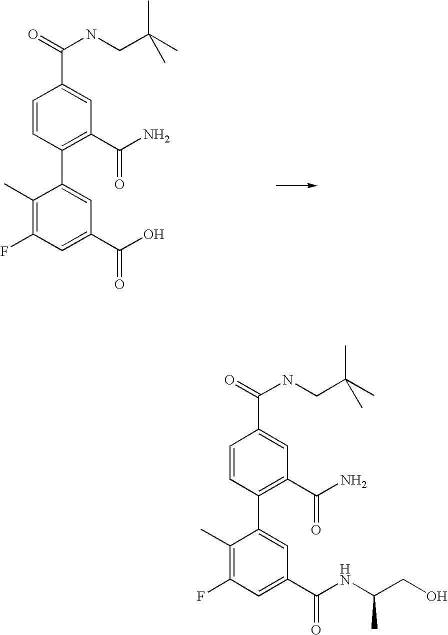

To a solution of 2′-(aminocarbonyl)-4′-{[(2,2-dimethylpropyl)amino]carbonyl}-5-fluoro-6-methyl-3-biphenylcarboxylic acid (23.2 mg, 0.06 mmol) in DMF (0.6 mL) was added HBTU (25.0 mg, 0.066 mmol), Et₃N (16.9 uL, 0.12 mmol) and (2R)-2-amino-1-propanol (9.7 uL, 0.09 mmol). The solution was stirred at room temperature over night. The reaction mixture was quenched with H₂O (1 drop), DMSO (0.5 mL), concentrated and filtered. Purification via HPLC (Gilson) then afforded the title compound (15 mg, 57%). LC-MS m/z 444 (M+H)⁺.

### Example 76

### N⁴-(2,2-dimethylpropyl)-5′-fluoro-N³′-[(4-fluorophenyl)methyl]-6′-methyl-2,3′,4-biphenyltricarboxamide

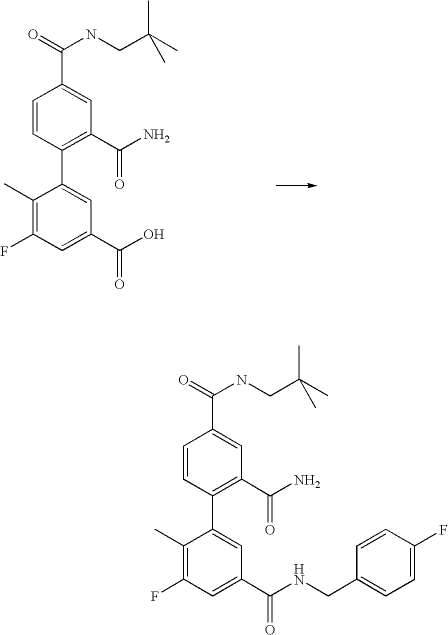

To a solution of 2′-(aminocarbonyl)-4′-{[(2,2-dimethylpropyl)amino]carbonyl}-5-fluoro-6-methyl-3-biphenylcarboxylic acid (23.2 mg, 0.06 mmol) in DMF (0.6 mL) was added HBTU (25.0 mg, 0.066 mmol), Et₃N (16.9 uL, 0.12 mmol) and [(4-fluorophenyl)methyl]amine (10.3 uL, 0.09 mmol). The solution was stirred at room temperature over night. The reaction mixture was quenched with H₂O (1 drop), DMSO (0.5 mL), concentrated and filtered. Purification via HPLC (Gilson) then afforded the title compound (19 mg, 65%). LC-MS m/z 494 (M+H)⁺.

### Example 77

### N³-cyclopropyl-N⁴′-(2,2-dimethylpropyl)-5-fluoro-6-methyl-2′-({[(phenylmethyl)amino]carbonyl}amino)-3,4′-biphenyldicarboxamide

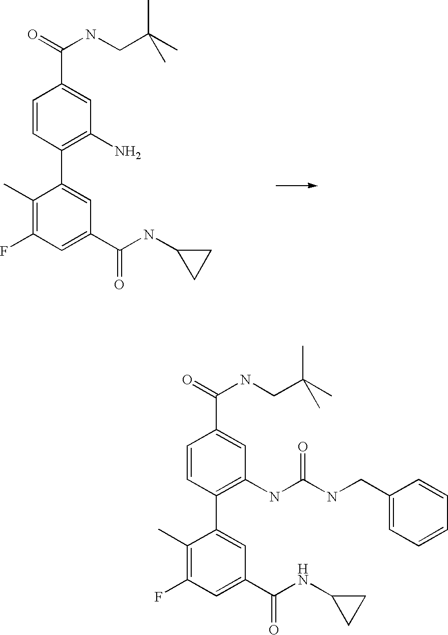

To a solution of 2′-amino-N³-cyclopropyl-N⁴′-(2,2-dimethylpropyl)-5-fluoro-6-methyl-3,4′-biphenyldicarboxamide (27.8 mg, 0.07 mmol) in DMF (0.49 mL) was added benzyl isocyanate (13.0 uL, 0.105 mmol) and Et₃N (19.7 uL, 0.14 mmol). The solution was stirred at room temperature over night. The reaction mixture was quenched with H₂O (1 drop), concentrated and filtered. Purification via HPLC (Gilson) then afforded the title compound (4 mg, 11%). LC-MS m/z 531 (M+H)⁺.

### Example 78

### methyl 4-{[(2,2-dimethylpropyl)amino]carbonyl}-5′-{[(2-hydroxyethyl)amino]carbonyl}-2′-methyl-2-biphenylcarboxylate

### 78a) 4′-{[(2,2-dimethylpropyl)amino]carbonyl}-6-methyl-2′-[(methyloxy)carbonyl]-3-biphenylcarboxylic acid

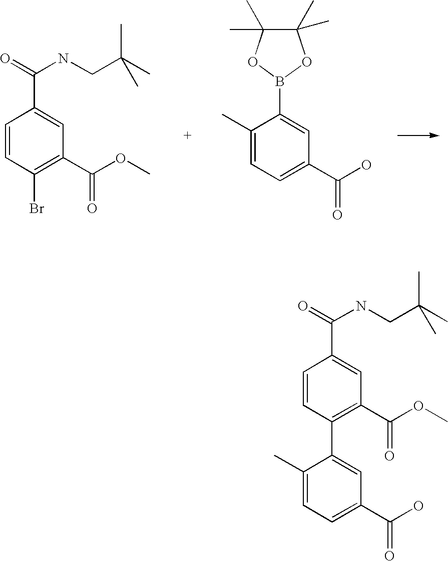

To a solution of methyl 2-bromo-5-{[(2,2-dimethylpropyl)amino]-carbonyl}benzoate (492 mg, 1.50 mmol) (example 10b) and 3-{hydroxy[(1,1,2,2-tetramethylpropyl)oxy]boranyl}-4-methylbenzoic acid (590 mg, 2.25 mmol) (for prep, see WO 2003/032970 exp. 1c) in H₂O (5.0 mL) and dioxane (15.0 mL) was added potassium carbonate (0.83 g, 6.0 mmol) and Pd(PPh₃)₄ (87.0 mg, 0.075 mmol). The solution was microwaved at 150° C. for 15 min. The reaction mixture was added with acetic acid (1 mL), concentrated and filtered. Purification via HPLC (Gilson) then afforded the title compound (445 mg, 78%). LC-MS m/z 384 (M+H)⁺.

### 78b) methyl 4-{[(2,2-dimethylpropyl)amino]carbonyl}-5′-{[(2-hydroxyethyl)amino]carbonyl}-2′-methyl-2-biphenylcarboxylate

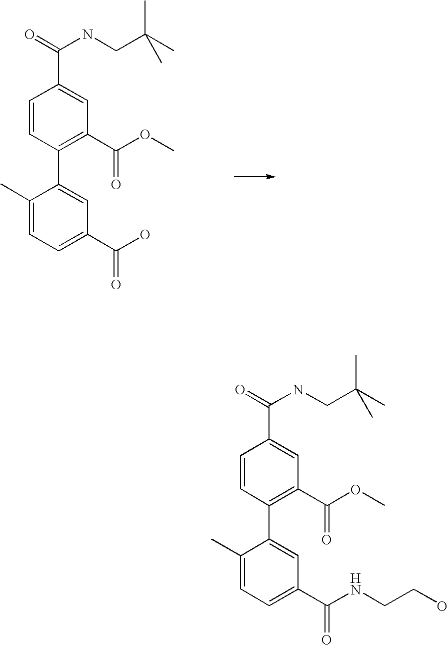

To a solution of 4′-{[(2,2-dimethylpropyl)amino]carbonyl}-6-methyl-2′-[(methyloxy)carbonyl]-3-biphenylcarboxylic acid (268 mg, 0.70 mmol) in CH₂Cl₂ (7.0 mL) was added EDC (148 mg, 0.77 mmol), HOBT (9.5 mg, 0.07 mmol), Et₃N (0.197 mL, 1.40 mmol) and 2-aminoethanol (63.2 uL, 1.05 mmol). The solution was stirred at room temperature over night. The reaction mixture was concentrated and filtered. Purification via HPLC (Gilson) then afforded the title compound (104 mg, 35%). LC-MS m/z 427 (M+H)⁺.

### Example 79

### methyl 5′-[(cyclopropylamino)carbonyl]-4-{[(2,2-dimethylpropyl)amino]carbonyl}-2′-methyl-2-biphenylcarboxylate

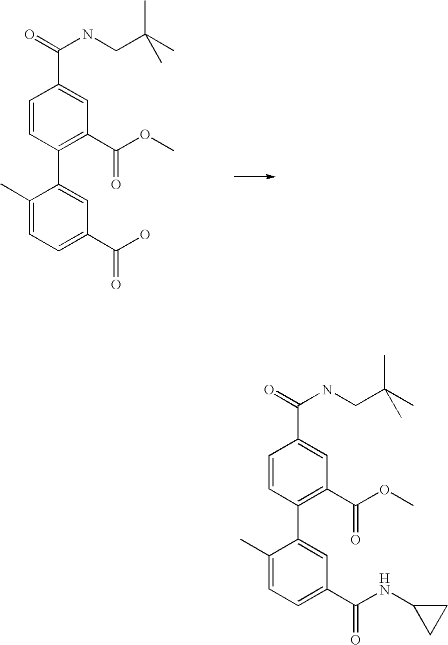

To a solution of 4′-{[(2,2-dimethylpropyl)amino]carbonyl}-6-methyl-2′-[(methyloxy)carbonyl]-3-biphenylcarboxylic acid (575 mg, 1.50 mmol) in CH₂Cl₂ (15.0 mL) was added EDC (316 mg, 1.65 mmol), HOBT (20.3 mg, 0.15 mmol), Et₃N (0.422 mL, 3.0 mmol) and cyclopropanamine (156 uL, 2.25 mmol). The solution was stirred at room temperature over night. The reaction mixture was concentrated and filtered. Purification via combiflash then afforded the title compound (306 mg, 48%). LC-MS m/z 423 (M+H)⁺.

### Example 80

### 5′-[(cyclopropylamino)carbonyl]-4-{[(2,2-dimethylpropyl)amino]carbonyl}-2′-methyl-2-biphenylcarboxylic acid

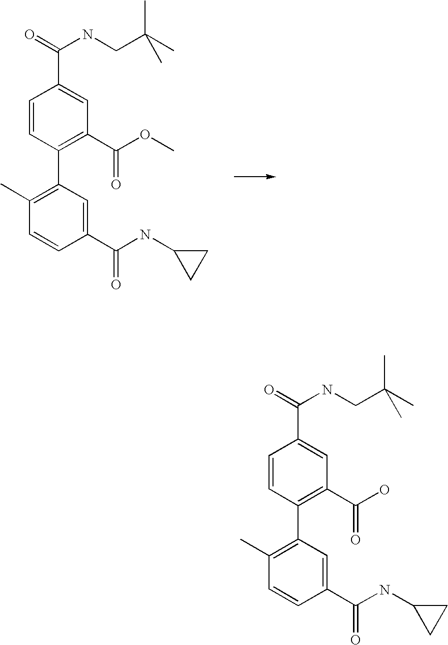

To a solution of methyl 5′-[(cyclopropylamino)carbonyl]-4-{[(2,2-dimethylpropyl)amino]carbonyl}-2′-methyl-2-biphenylcarboxylate (270 mg, 0.639 mmol) in H₂O (1.25 mL) and MeOH (1.25 mL) was added potassium hydroxide (107 mg, 1.92 mmol). The solution was microwaved at 100° C. for 10 min. The reaction mixture was neutralized with acetic acid, concentrated and filtered. Purification via combiflash then afforded the title compound (261 mg, 100%). LC-MS m/z 409 (M+H)⁺.

### Example 81

### N³′-cyclopropyl-N⁴-(2,2-dimethylpropyl)-6′-methyl-2,3′,4-biphenyltricarboxamide

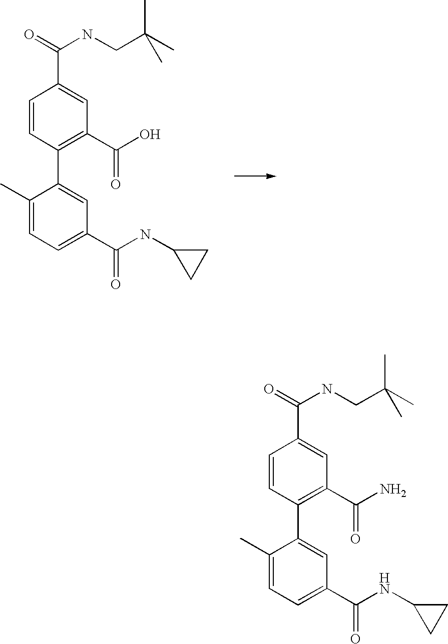

To a solution of 5′-[(cyclopropylamino)carbonyl]-4-{[(2,2-dimethylpropyl)amino]carbonyl}-2′-methyl-2-biphenylcarboxylic acid (30 mg, 0.073 mmol) in DMF (0.73 mL) was added HBTU (33.4 mg, 0.088 mmol), Et₃N (20.7 uL, 0.15 mmol) and ammonia (2.0 M in MeOH, 0.147 mL, 0.294 mmol). The solution was stirred at room temperature over night. The reaction mixture was quenched with H₂O (1 drop), DMSO (0.5 mL), concentrated and filtered. Purification via HPLC (Gilson) then afforded the title compound (27 mg, 90%). LC-MS m/z 408 (M+H)⁺.

### Example 82

### N³′-cyclopropyl-N⁴-(2,2-dimethylpropyl)-6′-methyl-N²-1,3-thiazol-2-yl-2,3′,4-biphenyltricarboxamide

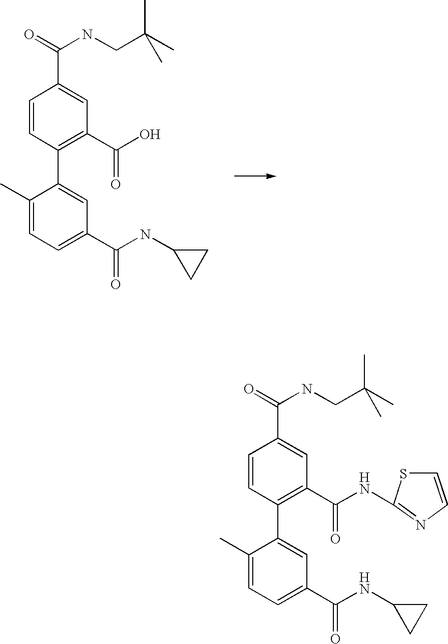

To a solution of 5′-[(cyclopropylamino)carbonyl]-4-{[(2,2-dimethylpropyl)amino]carbonyl}-2′-methyl-2-biphenylcarboxylic acid (30 mg, 0.073 mmol) in DMF (0.73 mL) was added HBTU (33.4 mg, 0.088 mmol), Et₃N (20.7 uL, 0.15 mmol) and 1,3-thiazol-2-amine (14.7 mg, 0.147 mmol). The solution was stirred at room temperature over night. The reaction mixture was quenched with H₂O (1 drop), DMSO (0.5 mL), concentrated and filtered. Purification via HPLC (Gilson) then afforded the title compound (13 mg, 36%). LC-MS m/z 491 (M+H)⁺.

### Example 83

### N³′-cyclopropyl-N⁴-(2,2-dimethylpropyl)-N²-(3-hydroxypropyl)-6′-methyl-2,3′,4-biphenyltricarboxamide

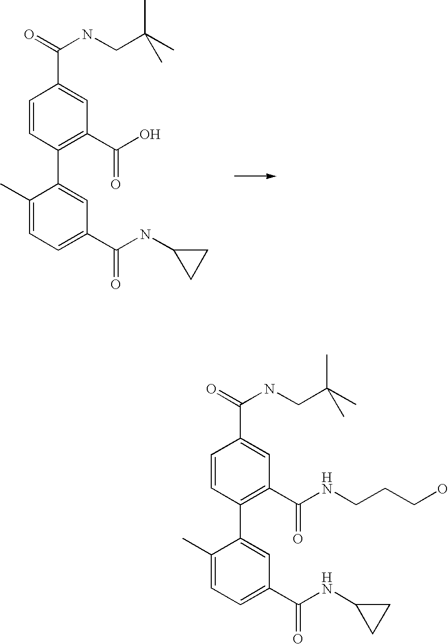

To a solution of 5′-[(cyclopropylamino)carbonyl]-4-{[(2,2-dimethylpropyl)amino]carbonyl}-2′-methyl-2-biphenylcarboxylic acid (30 mg, 0.073 mmol) in DMF (0.73 mL) was added HBTU (33.4 mg, 0.088 mmol), Et₃N (20.7 uL, 0.15 mmol) and 3-amino-1-propanol (11.2 uL, 0.147 mmol). The solution was stirred at room temperature over night. The reaction mixture was quenched with H₂O (1 drop), DMSO (0.5 mL), concentrated and filtered. Purification via HPLC (Gilson) then afforded the title compound (16 mg, 47%). LC-MS m/z 466 (M+H)⁺.

### Example 84

### N³′-cyclopropyl-N⁴-(2,2-dimethylpropyl)-6′-methyl-N²-[(4-methylphenyl)methyl]-2,3′,4-biphenyltricarboxamide

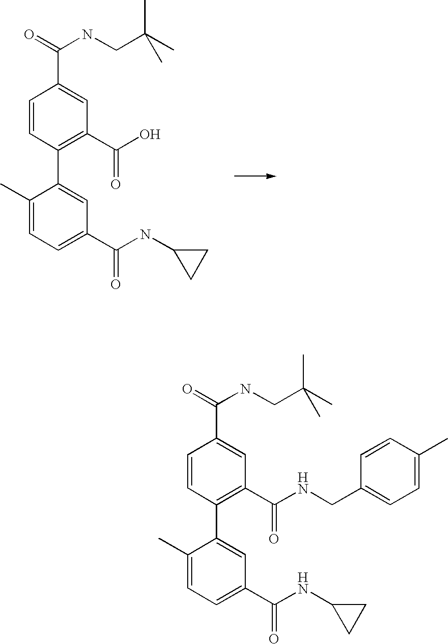

To a solution of 5′-[(cyclopropylamino)carbonyl]-4-{[(2,2-dimethylpropyl)amino]carbonyl}-2′-methyl-2-biphenylcarboxylic acid (30 mg, 0.073 mmol) in DMF (0.73 mL) was added HBTU (33.4 mg, 0.088 mmol), Et₃N (20.7 uL, 0.15 mmol) and [(4-methylphenyl)methyl]amine (18.6 uL, 0.147 mmol). The solution was stirred at room temperature over night. The reaction mixture was quenched with H₂O (1 drop), DMSO (0.5 mL), concentrated and filtered. Purification via HPLC (Gilson) then afforded the title compound (16 mg, 43%). LC-MS m/z 512 (M+H)⁺.

### Example 85

### N³′-cyclopropyl-N⁴-(2,2-dimethylpropyl)-6′-methyl-N²-3-pyridinyl-2,3′,4-biphenyltricarboxamide

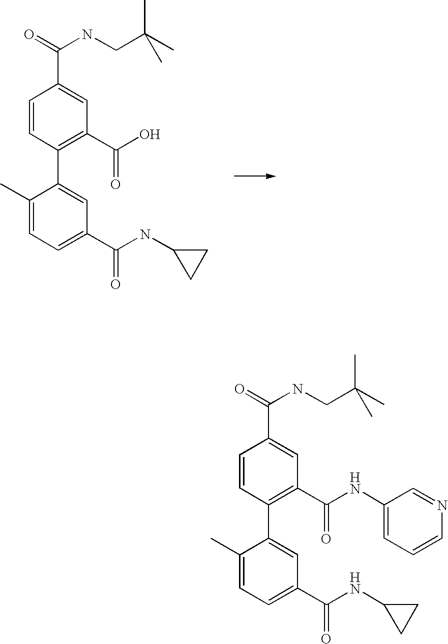

To a solution of 5′-[(cyclopropylamino)carbonyl]-4-{[(2,2-dimethylpropyl)amino]carbonyl}-2′-methyl-2-biphenylcarboxylic acid (30 mg, 0.073 mmol) in DMF (0.73 mL) was added HBTU (33.4 mg, 0.088 mmol), Et₃N (20.7 uL, 0.15 mmol) and 3-pyridinamine (13.8 mg, 0.147 mmol). The solution was stirred at room temperature over night. The reaction mixture was quenched with H₂O (1 drop), DMSO (0.5 mL), concentrated and filtered. Purification via HPLC (Gilson) then afforded the title compound (10 mg, 28%). LC-MS m/z 485 (M+H)⁺.

### Example 86

### 4-{[(2,2-dimethylpropyl)amino]carbonyl}-5′-{[(2-hydroxyethyl)amino]carbonyl}-2′-methyl-2-biphenylcarboxylic acid

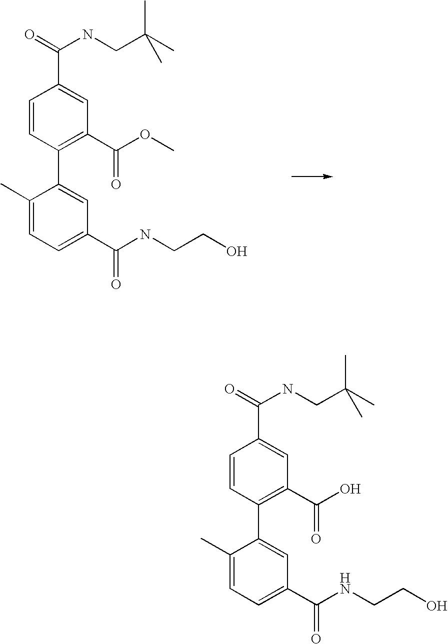

To a solution of methyl 4-{[(2,2-dimethylpropyl)amino]carbonyl}-5′-{[(2-hydroxyethyl)amino]carbonyl}-2′-methyl-2-biphenylcarboxylate (95 mg, 0.223 mmol) (example 87b) in MeOH (1.1 mL) was added ammonia hydroxide (concentrate in H₂O, 1.1 mL) and zinc iodine (3.6 mg, 0.011 mmol). The solution was microwaved at 100° C. for 13 hr. The reaction mixture was acidified with acetic acid (0.5 mL), concentrated and filtered. Purification via HPLC (Gilson) then afforded the title compound (72 mg, 78%). LC-MS m/z 413 (M+H)⁺.

### Example 87

### N₄-(2,2-dimethylpropyl)-N³′-(2-hydroxyethyl)-6′-methyl-2,3′,4-biphenyltricarboxamide

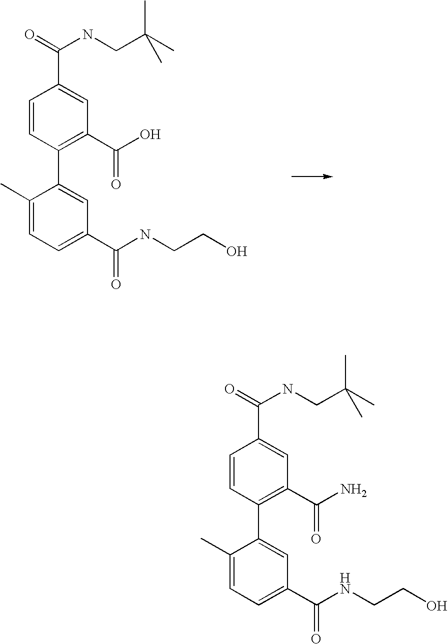

To a solution of 4-{[(2,2-dimethylpropyl)amino]carbonyl}-5′-{[(2-hydroxyethyl)amino]carbonyl}-2′-methyl-2-biphenylcarboxylic acid (72 mg, 0.175 mmol) in DMF (1.75 mL) was added HBTU (79.6 mg, 0.21 mmol), Et₃N (49 uL, 0.349 mmol) and ammonia (2.0 M in MeOH, 0.175 mL, 0.349 mmol). The solution was stirred at room temperature over night. The reaction mixture was quenched with H₂O (1 drop), DMSO (0.5 mL), concentrated and filtered. Purification via HPLC (Gilson) then afforded the title compound (11 mg, 15%). LC-MS m/z 412 (M+H)⁺.

### Example 88

### N⁴-(2,2-dimethylpropyl)-5′-fluoro-N³′-(2-hydroxyethyl)-6′-methyl-2,3′,4-biphenyltricarboxamide

### 88a) (5-{[(1,1-dimethylethyl)oxy]carbonyl}-3-fluoro-2-methylphenyl)boronic acid

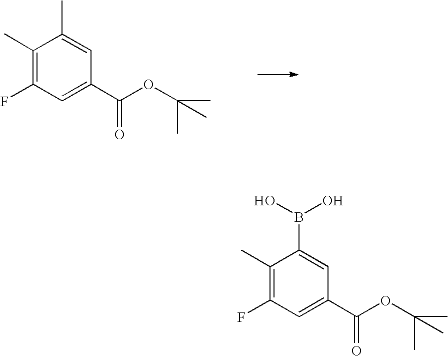

1,1-dimethylethyl 3-fluoro-5-iodo-4-methylbenzoate (54 g, 0.1607 moles, 1 eq) was dissolved in anhydrous THF (1080 mL) and cooled to −10 degrees. To this was added dropwise over 10 min isopropylmagnesium chloride (2 M in THF) (88.39 mL, 0.1768 moles, 1.1 eq), and the temperature was maintained below 0° C. The r×n mix was left for 1.5 hr, after which a further 10 mL (0.02 moles) of iPrMgCl was added. Trimethyl borate (35.9 mL, 0.321 moles, 2.0 eq) was then added dropwise and the resulting mixture left for 5 min (temperature maintained below 0° C.). The r×n mix was then quenched with water (400 mL) and ethyl acetate (500 mL) added. The layers were separated and water (100 mL) was added, after which it was basified with 2 M NaOH. The layers were separated and the aqueous acidified with 2 M HCl, after which, a solid precipitate was collected by filtration and dried at the pump to yield the title compound (3.75 g). To the organic layer was then added ethyl acetate (500 mL), and the precipitate was filtered*. This was suspended/dissolved in 3:2 water:THF (500 mL). This suspension was acidified using 2 M HCl. The suspension was then vacued until all the volatile solvent had been removed. The precipitate was filtered and dried at the pump to yield the title compound (14.75 g). The filtrate* was vacued and resuspended in ethyl acetate and basified using 2 M NaOH. The layers were separated and the aqueous acidified using 2 M HCl. This was filtered and dried at the pump to yield the title compound (1.5 g). The three batches were combined and dried in the oven (24.85 g). ¹H-NMR (MeOD) δ 7.65 (s, 1H), 7.50 (d, 1H), 2.28 (s, 3H), 1.57 (s, 9H).

### 88b) 3′-(1,1-dimethylethyl) 2-methyl 4-{[(2,2-dimethylpropyl)amino]carbonyl}-5′-fluoro-6′-methyl-2,3′-biphenyldicarboxylate

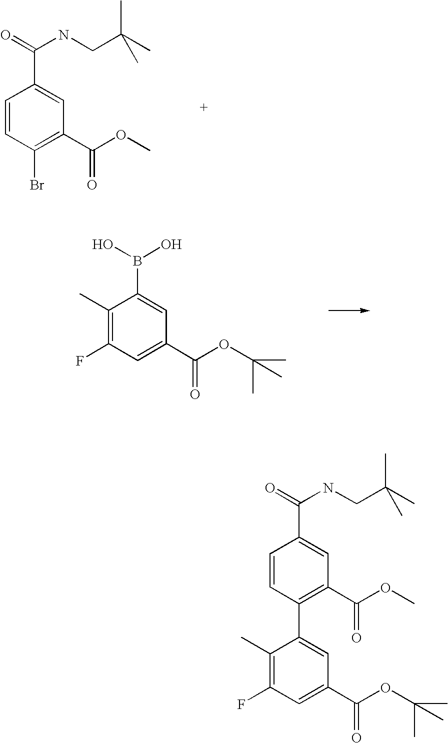

To a solution of methyl 2-bromo-5-{[(2,2-dimethylpropyl)amino]carbonyl}-benzoate (492 mg, 1.50 mmol) and (5-{[(1,1-dimethylethyl)oxy]carbonyl}-3-fluoro-2-methylphenyl)boronic acid (572 mg, 2.25 mmol) in H₂O (5.0 mL) and dioxane (15.0 mL) were added potassium carbonate (1.04 g, 7.50 mmol) and Pd(PPh₃)₄ (87.0 mg, 0.075 mmol). The solution was microwaved at 150° C. for 15 min. The reaction mixture was mixed with acetic acid (1 mL), concentrated and filtered. Purification via combiflash then afforded the title compound (319 mg, 46%). LC-MS m/z 458 (M+H)⁺.

### 88c) 5′-{[(1,1-dimethylethyl)oxy]carbonyl}-4-{[(2,2-dimethylpropyl)amino]-carbonyl}-3′-fluoro-2′-methyl-2-biphenylcarboxylic acid

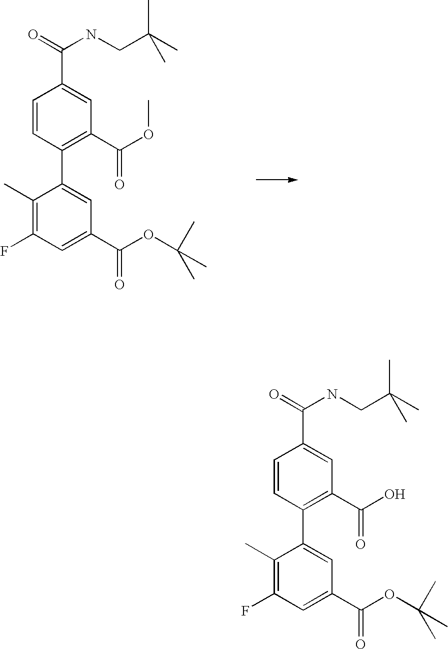

To a solution of 3′-(1,1-dimethylethyl) 2-methyl 4-{[(2,2-dimethylpropyl)amino]carbonyl}-5′-fluoro-6′-methyl-2,3′-biphenyldicarboxylate (290 mg, 0.636 mmol) in H₂O (1.6 mL) and MeOH (3.2 mL) was added potassium hydroxide (71.4 mg, 1.27 mmol). The solution was microwaved at 80° C. for 10 min. The reaction mixture was neutralized with acetic acid, concentrated and filtered. Purification via combiflash then afforded the title compound (224 mg, 80%). LC-MS m/z 444 (M+H)⁺.

### 88d) 1,1-dimethylethyl 2′-(aminocarbonyl)-4′-{[(2,2-dimethylpropyl)amino]carbonyl}-5-fluoro-6-methyl-3-biphenylcarboxylate

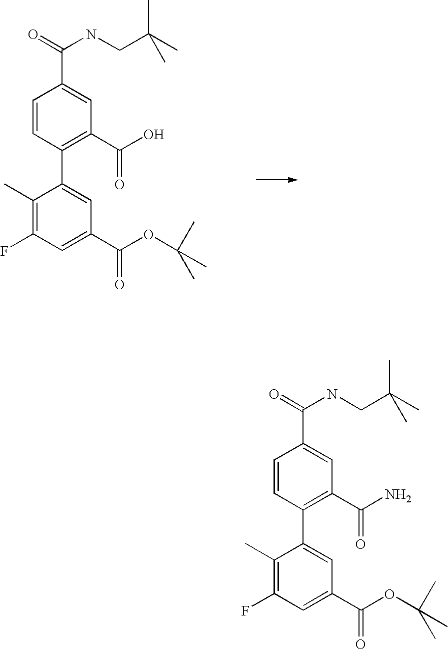

To a solution of 5′-{[(1,1-dimethylethyl)oxy]carbonyl}-4-{[(2,2-dimethylpropyl)amino]carbonyl}-3′-fluoro-2′-methyl-2-biphenylcarboxylic acid (1.33 g, 3.0 mmol) in THF (3.0 mL) were added Et₃N (0.464 mL, 3.3 mmol) and ethyl chloroformate (0.314 mL, 3.3 mmol) at 0° C. The solution was stirred for 15 min at the same temperature. To this solution was added ammonia hydroxide (28% solution in H₂O, 2.0 mL) in THF (2.0 mL) at 0° C. The solution was stirred at same temperature for 1 hr. The reaction mixture was concentrated and filtered. Purification via combiflash then afforded the title compound (0.481 g, 36%).

### 88e) 2′-(aminocarbonyl)-4′-{[(2,2-dimethylpropyl)amino]carbonyl}-5-fluoro-6-methyl-3-biphenylcarboxylic acid

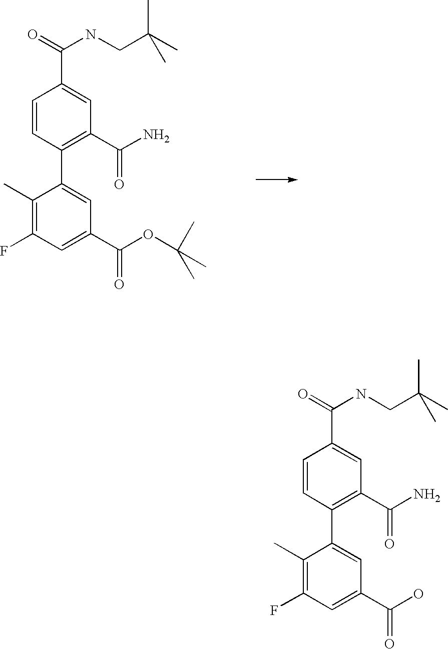

To a solution of 1,1-dimethylethyl 2′-(aminocarbonyl)-4′-{[(2,2-dimethylpropyl)amino]carbonyl}-5-fluoro-6-methyl-3-biphenylcarboxylate (0.48 g, 1.08 mmol) in CH₂Cl₂ (3.22 mL) was added triethylsilane (0.431 mL, 2.7 mmol) and TFA (1.09 mL, 14.1 mmol). The solution was stirred at room temperature for 1 hr then stored at −20° C. over night. The reaction mixture was concentrated. Washed with H₂O (50 mL), dried over sodium sulphate. Purification via combiflash then afforded the title compound (0.346 g, 83%).

### 88f) N⁴-(2,2-dimethylpropyl)-5′-fluoro-N³′-(2-hydroxyethyl)-6′-methyl-2,3′,4-biphenyltricarboxamide

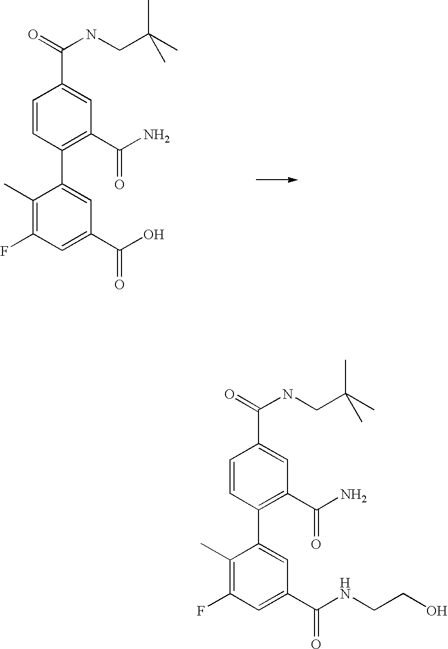

To a solution of 2′-(aminocarbonyl)-4′-{[(2,2-dimethylpropyl)amino]carbonyl}-5-fluoro-6-methyl-3-biphenylcarboxylic acid (30 mg, 0.078 mmol) in DMF (0.78 mL) were added HBTU (44 mg, 0.116 mmol), Et₃N (21.8 uL, 0.155 mmol) and 2-aminoethanol (7.0 uL, 0.116 mmol). The solution was stirred at room temperature over night. The reaction mixture was quenched with H₂O (1 drop), DMSO (0.5 mL), concentrated and filtered. Purification via HPLC (Gilson) then afforded the title compound (20 mg, 60%). MS (ES) m/z 430 (M+H)⁺.

### Example 89

### N⁴-(2,2-dimethylpropyl)-5′-fluoro-N³′-(3-hydroxypropyl)-6′-methyl-2,3′,4-biphenyltricarboxamide

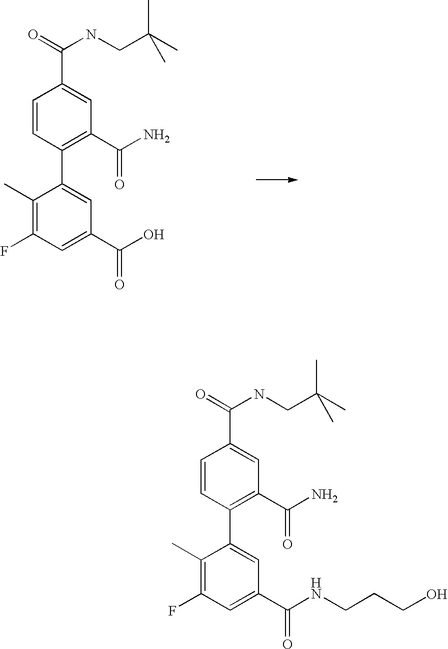

To a solution of 2′-(aminocarbonyl)-4′-{[(2,2-dimethylpropyl)amino]-carbonyl}-5-fluoro-6-methyl-3-biphenylcarboxylic acid (30 mg, 0.078 mmol) in DMF (0.78 mL) were added HBTU (44 mg, 0.116 mmol), Et₃N (21.8 uL, 0.155 mmol) and 3-amino-1-propanol (8.8 uL, 0.116 mmol). The solution was stirred at room temperature over night. The reaction mixture was quenched with H₂O (1 drop), DMSO (0.5 mL), concentrated and filtered. Purification via HPLC (Gilson) then afforded the title compound (24 mg, 70%). LC-MS m/z 444 (M+H)⁺.

### Example 90

### N⁴-(2,2-dimethylpropyl)-5′-fluoro-6′-methyl-N³′-3-pyridinyl-2,3′,4-biphenyltricarboxamide

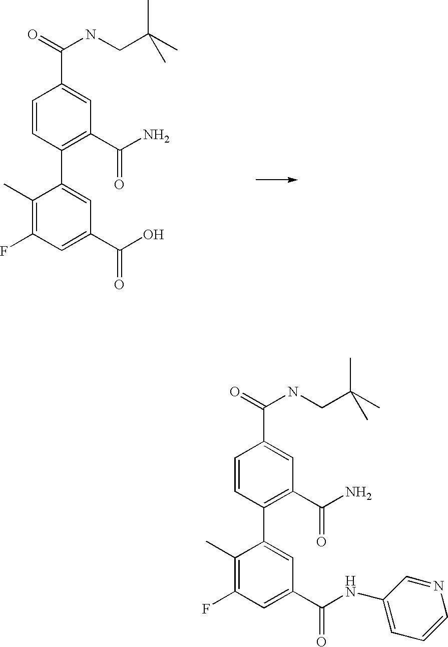

To a solution of 2′-(aminocarbonyl)-4′-{[(2,2-dimethylpropyl)amino]-carbonyl}-5-fluoro-6-methyl-3-biphenylcarboxylic acid (30 mg, 0.078 mmol) in DMF (0.78 mL) were added HBTU (44 mg, 0.116 mmol), Et₃N (21.8 uL, 0.155 mmol) and 3-pyridinamine (10.9 mg, 0.116 mmol). The solution was stirred at room temperature over night. The reaction mixture was quenched with H₂O (1 drop), DMSO (0.5 mL), concentrated and filtered. Purification via HPLC (Gilson) then afforded the title compound (14 mg, 37%). LC-MS m/z 483 (M+H)⁺.

### ABBREVIATIONS

Ac Acetyl
aq aqueous
ADDP 1,1-Azadicarbonyldipiperadine
Boc t-Butoxycarbonyl
Bu Butyl
CDI Carbonyldiimidazole
DCM Dichloromethane
DIPEA N,N-Diisopropylethylamine
DMAP 4-(Dimethylamino)pyridine
DMF Dimethylformamide
DMSO Dimethylsulfoxide
DPPA Diphenyl phosphoryl azide
EDC 1-(3-Dimethylaminopyopyl)-3-ethylcarbodiimide hydrochloride
EDCI 1-(3-dimethylaminopropyl)-3-ethylcarbodiimide hydrochloride
Et₃N Triethylamine
EtOH Ethanol
eq equivalents
equivs equivalents
g grams
h hours
Hal Halogen
HATU O-(7-Azabenzotriazol-1-yl)-N,N,N′,N′-tetramethyluronium hexafluorophosphate
HBTU O-Benzotriazole-N,N,N′,N′-tetramethyl-uronium-hexafluorophosphate
HOBT N-Hydroxybenzotriazol Anhydrous IPA isopropanol
iPr isopropyl
KOAC Potassium acetate
I or L liters
mdap Mass-directed autopreparative HPLC
MeOH Methanol
min Minutes
mg milligrams
mEq milliequivalents
ml millilitres
mol moles
mmol millimoles
mp or mpt melting point
PdCl₂dppf [1,1′-bis(diphenylphosphino)ferrocene]dichloropalladium(II) complex with dichloromethane (1:1)
Pd(PPh₃)₄ Tetrakis(triphenylphosphine)palladium(0)
Ph Phenyl
Rt Retention time
RT Room Temperature
rt room temperature
SPE Solid phase extraction
TLC Thin layer chromatography
THF Tetrahydrofuran

The application of which this description and claims forms part may be used as a basis for priority in respect of any subsequent application. The claims of such subsequent application may be directed to any feature or combination of features described herein. They may take the form of product, composition, process or use claims and may include, by way of example and without limitation, one or more of the following claims:

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore, the Examples herein are to be construed as merely illustrative and not a limitation of the scope of the present invention in any way. The embodiments of the invention in which an exclusive property or privilege is claimed are defined as follows.

1. A compound of formula (I): 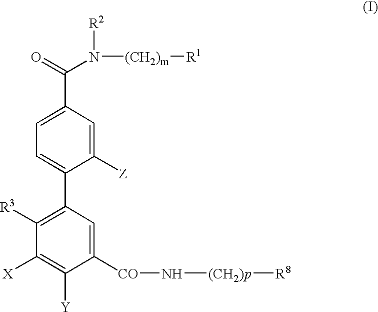
wherein
R¹ is selected from hydrogen; C₁₋₆alkyl optionally substituted by up to three groups independently selected from C₁₋₆alkoxy, halogen and hydroxy; C₃₋₇cycloalkyl optionally substituted independently by one or more C₁₋₁₆alkyl groups; an aryl, heteroaryl, or heterocyclic ring each optionally substituted, independently, by up to three groups selected from R⁵ and R⁶;
R² is hydrogen, C₁₋₆alkyl or a —(CH₂)ₚ—C₃₋₇cycloalkyl optionally substituted independently by one or more C₁₋₆alkyl groups,
or the (CH₂)ₘR¹ and R², together with the nitrogen atom to which they are bound, form an optionally substituted, four- to six-membered heterocyclic ring optionally containing another heteroatom selected from O/N/S;
R³ is halogen or methyl;
R⁴ is hydrogen, C₁₋₆ alkyl, halo-substituted-C₁₋₄ alkyl, or C₃₋₇cycloalkyl;
R⁵ is independently C₁₋₆alkyl, OR⁴, —(CH₂)ₚ—C₃₋₇cycloalkyl optionally substituted independently by one or more C₁₋₆alkyl groups, —CONR⁹R¹⁰, —NHCOR¹⁰, —SO₂NHR⁹, —(CH₂)_{q}NHSO₂R¹⁰, halogen, CN, —(CH₂)_{q}NR¹¹R¹², and trifluoromethyl;
R⁶ is independently hydrogen, C₁₋₆alkyl, OR⁴, halogen, trifluoromethyl and —(CH₂)_{q}NR¹¹R¹²;
R⁸ is selected from hydrogen, C₁₋₆alkyl, C₃₋₇cycloalkyl optionally substituted by one or more C₁₋₆alkyl groups, OH, C₁₋₆alkyl optionally substituted by one or more hydroxyl groups, CONHR⁹, phenyl optionally substituted by R¹³ and/or R¹⁴, or a heteroaryl optionally substituted by R¹³ and/or R¹⁴;
R⁹ and R¹⁰ are each independently selected from hydrogen and C₁₋₆alkyl, or
R⁹ and R¹⁰, together with the nitrogen atom to which they are bound, form a five- to six-membered heterocyclic ring optionally containing one additional heteroatom selected from oxygen, sulfur and N—R¹⁵, wherein the ring is optionally substituted by up to two C₁₋₆alkyl groups;
R¹¹ is selected from hydrogen, C₁₋₆alkyl and —(CH₂)ₚ—C₃₋₇cycloalkyl optionally substituted by one or more C₁₋₁₆alkyl groups,
R¹² is selected from hydrogen and C₁₋₆alkyl, or
R¹¹ and R¹², together with the nitrogen atom to which they are bound, form a five or six-membered heterocyclic ring optionally containing one additional heteroatom selected from oxygen, sulfur and N—R¹⁵;
R¹³ is selected from C₁₋₆alkyl, C₁₋₆alkoxy, —(CH₂)ₚ—C₃₋₇cycloalkyl optionally substituted by one or more C₁₋₆alkyl groups, —CONR⁹R¹⁰, —NHCOR¹⁰, halogen, CN, —(CH₂)_{q}NR¹¹R¹², trifluoromethyl, phenyl optionally substituted independently by one or more R¹⁴ groups, heterocyclic optionally substituted independently by one or more R¹⁴ groups, and a heteroaryl optionally substituted independently by one or more R¹⁴ groups;
R¹⁴ is selected from C₁₋₆alkyl, C₁₋₆alkoxy, halogen, halo-substituted C₁₋₄ alkyl, and NR¹¹R¹²;
R¹⁵ is selected from hydrogen and methyl;
X and Y are each independently selected from hydrogen, methyl and halogen;
Z is selected from —(CH₂)ₛCOOR¹⁶, or —(CH₂)ₛCONR¹⁶R¹⁷;
R¹⁶ and R¹⁷ are independently selected from hydrogen, optionally substituted C₁₋₆alkyl, —(CR₂₀R₂₁)ᵥOR¹⁸, —(CR₂₀R₂₁)ᵥNR¹⁸R¹⁹, —(CR₂₀R₂₁)ᵥNHSO₂R⁸, —(CR₂₀R₂₁)_{V}CONR¹⁸R¹⁹, —(CR₂₀R₂₁)ᵥCOOR¹⁸, optionally substituted —(CR₂₀R₂₁)ₜheteroaryl, optionally substituted —(CR₂₀R₂₁)ₜ aryl, optionally substituted —(CR₂₀R₂₁)ₜheterocyclic, optionally substituted —(CR₂₀R₂₁)ₜ C₃₋₇cycloalkyl, or optionally substituted —(CR₂₀R₂₁)ₜ C₃₋₇cycloalkenyl; or
R¹⁶ and R¹⁷, together with the nitrogen atom to which they are bound, form an optionally substituted five- to six-membered ring optionally containing at least one additional heteroatom selected from oxygen, sulfur and N—R¹⁵;
R¹⁸ and R¹⁹ are each independently selected from hydrogen and C₁₋₆alkyl optionally substituted by up to two hydroxy groups; or
R¹⁸ and R¹⁹, together with the nitrogen atom to which they are bound, form a five- to six-membered ring, optionally containing one additional heteroatom selected from oxygen, sulfur and N—R¹⁵, and wherein the ring is optionally substituted by up to two groups independently selected from oxo, halogen and C₁₋₆alkyl;
R₂₀ and R₂₁ are independently selected from hydrogen or C₁₋₄ alkyl;
m is 0 or an integer selected from 1, 2, 3 and 4;
p is 0 or an integer selected from 1 and 2;
q is 0 or an integer selected from 1, 2 and 3;
r is 0 or an integer of 1;
s is 0 or an integer selected from 1, 2, 3 and 4; and
t is 0 or an integer selected from 1, 2, 3, 4, 5 and 6;
v is an integer selected from 1, 2, 3, 4, 5 and 6;
or a pharmaceutically acceptable salt or derivative thereof.

2. The compound according to claim 1 wherein R¹ is selected from C₁₋₆alkyl, C₃₋₇cycloalkyl or phenyl optionally substituted by up to three groups selected independently from R⁵ and R⁶.

3. The compound according to claim 2 wherein R¹ is C₃₋₆cycloalkyl or C₁₋₆alkyl.

4. The compound according claim 1 wherein R¹ is a heteroaryl, or heterocyclic ring, each optionally substituted, independently, by up to three groups selected from R⁵ and R⁶.

5. The compound according to claim 1 wherein R² is hydrogen.

6. The compound according to claim 1 wherein m is 0 or 1.

7. The compound according to claim 1 wherein Z is (CH₂)ₛCONR¹⁶R¹⁷.

8. The compound according to claim 1 wherein Z is (CH₂)ₛCOOR¹⁶.

9. The compound according to claim 7 wherein s is 0.

10. The compound according to claim 1 wherein R¹⁶ is an optionally substituted hydrogen, C₁₋₆alkyl, —(CR₂₀R₂₁)ᵥOR¹⁸, or —(CR₂₀R₂₁)ᵥNR¹⁸R¹⁹.

11. The compound according to claim 10 wherein R¹⁶ is hydrogen, C₁₋₆alkyl, C₁₋₆alkyl optionally substituted one or more times independently by hydroxyl, halogen, C₁₋₆alkoxy, and NR₇R_{7′}, wherein R₇ and R_{7′} are each independently hydrogen or C₁₋₄ alkyl.

12. The compound according to claim 11 wherein R¹⁶ is propyl, isopropyl, 2-hydroxypropyl, 3-hydroxypropyl, 2,2,2-trifluoroethyl, dimethylamino)ethyl, hydrogen, 3-(ethyloxy)propyl, 5-hydroxypentyl, (dibutylamino)propyl, or 1-(methylethyl)oxy)propyl.

13. The compound according to claim 1 wherein R¹⁶ is an optionally substituted —(CR₂₀R₂₁)ₜheteroaryl, optionally substituted —(CR₂₀R₂₁)ₜ aryl, or an optionally substituted —(CR₂₀R₂₁)ₜheterocyclic.

14. The compound according to claim 12 wherein R¹⁶ is an optionally substituted thiazolyl, optionally substituted phenyl, optionally substituted pyridine, optionally substituted imidazole, optionally substituted piperidinyl, optionally substituted piperazinyl, optionally substituted phenylC₁₋₆alkyl, or optionally substituted pyrrolidinyl C₁₋₆alkyl.

15. The compound according to claim 12 wherein R¹⁶ is 1,3-thiazolyl, optionally substituted phenyl, pyridine, imidazole, piperidinyl, piperazinyl, benzyl, phenylbutyl, phenylethyl, pyrrolidinylethyl, pyrrolidinylmethyl, or (4-methylphenyl)methyl, or (1-ethyl-2-pyrrolidinyl)methyl.

16. The compound according to claim 1 wherein R¹⁶ is 1,3-thiazolyl, and t is 0.

17. The compound according to claim 1 wherein R¹⁶ and R¹⁷, together with the nitrogen atom to which they are bound, form an optionally substituted five- to six-membered ring optionally containing at least one additional heteroatom selected from oxygen, sulfur and N—R¹⁵.

18. The compound according to claim 14 wherein R¹⁶ and R¹⁷ together form an optionally substituted piperidinyl or piperazinyl ring.

19. The compound according to claim 1 wherein R¹⁶ is an optionally substituted —(CR₂₀R₂₁)ₜ C₃₋₇cycloalkyl.

20. The compound according to claim 19 wherein R¹⁶ is cyclopropyl, cyclopentyl, or cyclohexylC1-6 alkyl.

21. The compound according to claim 1 wherein R⁸ is C₃₋₆cycloalkyl.

22. The compound according to claim 1 wherein R⁸ is cyclopropyl, p=0.

23. The compound according to claim 1 wherein R⁸ is C₁₋₆alkyl, OH, or a C₁₋₆alkyl optionally substituted by one or more hydroxyl groups.

24. The compound according to claim 1 wherein Z is (CH₂)ₛCONR¹⁶R¹⁷, R¹⁶ is 1,3-thiazolyl, t is 0, R⁸ is cyclopropyl, p=0.

25. The compound according to claim 24 wherein R² is hydrogen, R¹ is selected from C₁₋₆alkyl, or C₃₋₇cycloalkyl.

26. The compound according to claim 1 which is:
5′-[(Cyclopropylamino)carbonyl]-4-{[(2,2-dimethylpropyl)amino]carbonyl}-3′-fluoro-2′-methyl-2-biphenylcarboxylic acid; or
N²-[(1S)-1-Cyclohexylethyl]-N³′-cyclopropyl-N⁴-(2,2-dimethylpropyl)-5′-fluoro-6′-methyl-2,3′,4-biphenyltricarboxamide; or
N³-Cyclopropyl-N⁴′-(2,2-dimethylpropyl)-5-fluoro-2′-[(4-hydroxy-1-piperidinyl)carbonyl]-6-methyl-3,4′-biphenyldicarboxamide; or
N³′-Cyclopropyl-N⁴-(2,2-dimethylpropyl)-5′-fluoro-6′-methyl-N²-propyl-2,3′,4-biphenyltricarboxamide; or
N³′-Cyclopropyl-N²-[2-(dimethylamino)ethyl]-N⁴-(2,2-dimethylpropyl)-5′-fluoro-6′-methyl-2,3′,4-biphenyltricarboxamide; or
N³′-Cyclopropyl-N⁴-(2,2-dimethylpropyl)-5′-fluoro-6′-methyl-N²-1,3-thiazol-2-yl-2,3′,4-biphenyltricarboxamide; or
N³′-Cyclopropyl-N⁴-(2,2-dimethylpropyl)-5′-fluoro-6′-methyl-N²-(1-methylethyl)-2,3′,4-biphenyltricarboxamide; or
N³-Cyclopropyl-N⁴′-(2,2-dimethylpropyl)-5-fluoro-6-methyl-2′-[(3-oxo-1-piperazinyl)carbonyl]-3,4′-biphenyldicarboxamide; or
N³′-Cyclopropyl-N⁴-(2,2-dimethylpropyl)-5′-fluoro-N²-[(2S)-2-hydroxypropyl]-6′-methyl-2,3′,4-biphenyltricarboxamide; or
N³′-Cyclopropyl-N⁴-(2,2-dimethylpropyl)-5′-fluoro-N²-[(2R)-2-hydroxypropyl]-6′-methyl-2,3′,4-biphenyltricarboxamide; or a pharmaceutically acceptable salt, or derivative thereof.

27. The compound according to Examples 1 to 8, 10 to 76, and 77 to 90.

28. A pharmaceutical composition comprising a pharmaceutically acceptable carrier or diluent thereof, and a compound according to claim 1 or a pharmaceutically salt or derivative thereof.

29. A method for treating a condition or disease state mediated by p38 kinase activity or mediated by cytokines produced by the activity of p38 kinase comprising administering to a patient in need thereof a compound according to claim 1, or a pharmaceutically acceptable derivative thereof.

30. A process for preparing a compound according to claim 1, or a pharmaceutically acceptable derivative thereof, which comprises:
(a) reacting a compound of (II) 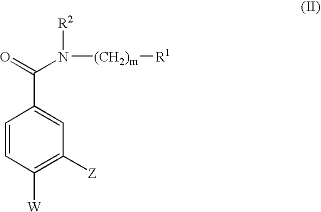
in which R¹, R², Z and m are as defined in claim 1 and W is bromine or iodine;
with a compound of formula (III) 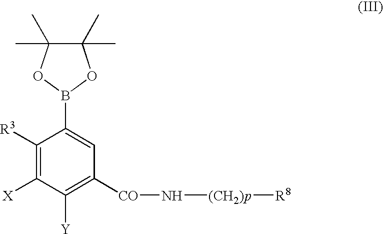
in which R³, R⁴, R⁸, p, X and Y are as defined in claim 1,
in the presence of a catalyst, or
(b) reacting a compound of formula (VIII) 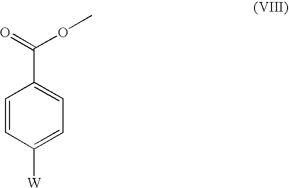
with a compound of formula (III) as hereinbefore defined and then reacting the acid thus formed after hydrolysis, if necessary, with an amine of formula (V) 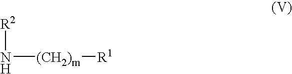
in which R¹, R² and m are as defined in claim 1,
under amide forming conditions
(c) reacting a compound of formula (II) as hereinbefore defined with a compound of formula (IX) 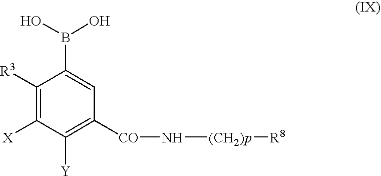
in which R³, R⁸, p, X and Y are as defined in claim 1,
in the presence of a catalyst,
(d) reacting a compound of formula (X) 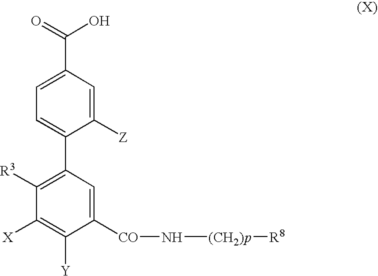
in which R³, R⁸, p, X, Y and Z are as defined in claim 1,
with an amine compound of formula (V) as defined above,
under amide forming conditions,
(e) final stage modification of one compound of formula (I) into another compound of formula (I), or
(e) conversion of a compound of formula (XII) 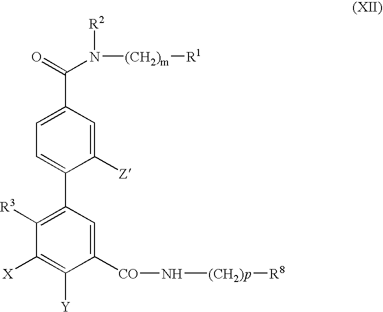
in which Z′ is a group convertible to Z as defined in claim 1.

31. A compound of formula (A): 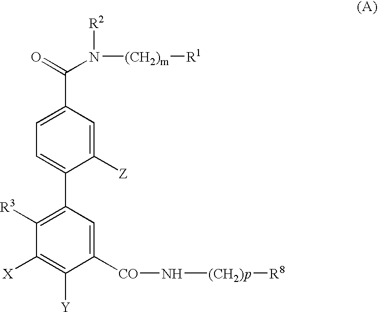
wherein
R¹ is selected from hydrogen; C₁₋₆alkyl optionally substituted by up to three groups independently selected from C₁₋₆alkoxy, halogen and hydroxy; C₃₋₇cycloalkyl optionally substituted independently by one or more C₁₋₆alkyl groups; an aryl, heteroaryl, or heterocyclic ring each optionally substituted, independently, by up to three groups selected from R⁵ and R⁶;
R² is hydrogen, C₁₋₆alkyl or a —(CH₂)ₚ—C₃₋₇cycloalkyl optionally substituted independently by one or more C₁₋₆alkyl groups,
or the (CH₂)ₘR¹ and R², together with the nitrogen atom to which they are bound, form an optionally substituted, four- to six-membered heterocyclic ring optionally containing another heteroatom selected from O/N/S;
R³ is halogen or methyl;
R⁴ is hydrogen, C₁₋₆ alkyl, halo-substituted-C₁₋₄ alkyl, or C₃₋₇cycloalkyl;
R⁵ is independently C₁₋₆alkyl, OR⁴, —(CH₂)ₚ—C₃₋₇cycloalkyl optionally substituted independently by one or more C₁₋₆alkyl groups, —CONR⁹R¹⁰, —NHCOR¹⁰, —SO₂NHR⁹, —(CH₂)_{q}NHSO₂R¹⁰, halogen, CN, —(CH₂)_{q}NR¹¹R¹², and trifluoromethyl;
R⁶ is independently hydrogen, C₁₋₆alkyl, OR⁴, halogen, trifluoromethyl and —(CH₂)_{q}NR¹¹R¹²;
R⁸ is selected from hydrogen, C₁₋₆alkyl, C₃₋₇cycloalkyl optionally substituted by one or more C₁₋₆alkyl groups, OH, C₁₋₆alkyl optionally substituted by one or more hydroxyl groups, CONHR⁹, phenyl optionally substituted by R¹³ and/or R¹⁴, or a heteroaryl optionally substituted by R¹³ and/or R¹⁴;
R⁹ and R¹⁰ are each independently selected from hydrogen and C₁₋₆alkyl, or
R⁹ and R¹⁰, together with the nitrogen atom to which they are bound, form a five- to six-membered heterocyclic ring optionally containing one additional heteroatom selected from oxygen, sulfur and N—R¹⁵, wherein the ring is optionally substituted by up to two C₁₋₆alkyl groups;
R¹¹ is selected from hydrogen, C₁₋₆alkyl and —(CH₂)ₚ—C₃₋₇cycloalkyl optionally substituted by one or more C₁₋₁₆alkyl groups,
R¹² is selected from hydrogen and C₁₋₆alkyl, or
R¹¹ and R¹², together with the nitrogen atom to which they are bound, form a five or six-membered heterocyclic ring optionally containing one additional heteroatom selected from oxygen, sulfur and N—R¹⁵;
R¹³ is selected from C₁₋₆alkyl, C₁₋₆alkoxy, —(CH₂)ₚ—C₃₋₇cycloalkyl optionally substituted by one or more C₁₋₆alkyl groups, —CONR⁹R¹⁰, —NHCOR¹⁰, halogen, CN, —(CH₂)_{q}NR¹¹R¹², trifluoromethyl, phenyl optionally substituted independently by one or more R¹⁴ groups, heterocyclic optionally substituted independently by one or more R¹⁴ groups, and a heteroaryl optionally substituted independently by one or more R¹⁴ groups;
R¹⁴ is selected from C₁₋₆alkyl, C₁₋₆alkoxy, halogen, halo-substituted C₁₋₄ alkyl, and NR¹¹R¹²;
R¹⁵ is selected from hydrogen and methyl;
X and Y are each independently selected from hydrogen, methyl and halogen;
Z is selected from —(CH₂)ₛNH₂, or (CH₂)ₛN(R²²)CONR²³R²⁴;
R²³ and R²⁴ are independently selected from hydrogen, optionally substituted C₁₋₆alkyl, (CR₂₀R₂₁)ᵥOR²⁵, (CR₂₀R₂₁)ᵥNR²⁵R²⁶, (CR₂₀R₂₁)ᵥNHSO₂R²⁵, (CR₂₀R₂₁)ᵥCONR²⁵R²⁶, (CR₂₀R₂₁)ᵥCOOR²⁵, optionally substituted (CR₂₀R₂₁)ₜheteroaryl, optionally substituted (CR₂₀R₂₁)ₜaryl, optionally substituted (CR₂₀R₂₁)ₜheterocyclic, optionally substituted (CR₂₀R₂₁)ₜ C₃₋₇cycloalkyl, or optionally substituted (CR₂₀R₂₁)ₜ C₃₋₇cycloalkenyl; or
R²³ and R²⁴, together with the nitrogen atom to which they are bound, form an optionally substituted five- to six-membered ring optionally containing at least one additional heteroatom selected from oxygen, sulfur and N—R¹⁵;
R²⁵ and R²⁶ are each independently selected from hydrogen and C₁₋₆alkyl optionally substituted by up to two hydroxy groups; or
R²⁵ and R²⁶, together with the nitrogen atom to which they are bound, form a five- to six-membered ring, optionally containing one additional heteroatom selected from oxygen, sulfur and N—R¹⁵, and wherein the ring is optionally substituted by up to two groups independently selected from oxo, halogen and C₁₋₆alkyl;
R₂₀ and R₂₁ are independently selected from hydrogen or C₁₋₄ alkyl;
R₂₂ is hydrogen or C₁₋₄ alkyl;
m is 0 or an integer selected from 1, 2, 3 and 4;
p is 0 or an integer selected from 1 and 2;
q is 0 or an integer selected from 1, 2 and 3;
r is 0 or an integer of 1;
s is 0 or an integer selected from 1, 2, 3 and 4; and
t is 0 or an integer selected from 1, 2, 3, 4, 5 and 6;
v is an integer selected from 1, 2, 3, 4, 5 and 6;
or a pharmaceutically acceptable salt or derivative thereof.

32. The compound according to Examples 9 and 76.

33. A pharmaceutical composition comprising a compound according to claim 31 or a pharmaceutically derivative thereof, and a pharmaceutically acceptable carrier or diluent thereof.

34. A method for treating a condition or disease state mediated by p38 kinase activity or mediated by cytokines produced by the activity of p38 kinase comprising administering to a patient in need thereof a compound according to claim 31, or a pharmaceutically acceptable derivative thereof.

35. The compound according to claim 8 wherein s is 0.
